(19) United States
(12) Patent Application Publication
(10) Pub. No.: US 2012/0219602 A1
(43) Pub. Date: Aug. 30, 2012

(54) METHODS FOR TREATING HERPES VIRUS INFECTIONS

(75) Inventors: Mary R. Flack, Ann Arobr, MI (US); Susan Marie Ciotti, Ann Arbor, MI (US); Tarek Hamouda, Milan, MI (US); Joyce A. Sutcliffe, West Newton, MA (US); James R. Baker, JR., Ann Arbor, MI (US)

(73) Assignee: NanoBio Corporation

(21) Appl. No.: 13/407,695

(22) Filed: Feb. 28, 2012

Related U.S. Application Data
(63) Continuation of application No. 12/425,984, filed on Apr. 17, 2009, now abandoned.
Application No. 12/425,984 is a continuation-in-part of application No. 11/080,629, filed on Mar. 16, 2005.
Application No. 11/080,629 is a continuation of application No. 11/067,626, filed on Feb. 28, 2005, now abandoned.
Application No. 11/067,626 is a continuation of application No. 10/860,582, filed on Jun. 4, 2004, now abandoned.
(60) Provisional application No. 61/046,262, filed on Apr. 18, 2008.
(60) Provisional application No. 60/475,633, filed on Jun. 4, 2003.

(51) Int. Cl. A61K 9/107; A61P 31/22; A61K 31/20; A61K 31/4164; B82Y 5/00

(52) U.S. Cl. 424/400; 514/558; 514/399; 977/788; 977/915

(57) ABSTRACT

The present invention relates to methods for treating, killing, and/or inhibiting the growth of Herpes viruses in human subjects comprising topically administering to a human subject in need thereof a nanoemulsion composition having antiviral properties.

### CROSS-REFERENCE TO RELATED APPLICATION

This application is a continuation of U.S. patent application Ser. No. 12/425,984, filed Apr. 17, 2009, which claims priority from U.S. Provisional Patent Application No. 61/046,262, filed Apr. 18, 2008. This application is also a continuation-in-part of U.S. patent application Ser. No. 11/080,629, filed Mar. 16, 2005, which is a continuation of U.S. patent application Ser. No. 11/067,626, filed Feb. 28, 2005, now abandoned, which is a continuation of U.S. patent application Ser. No. 10/860,582, filed Jun. 4, 2004, now abandoned and claims priority from U.S. Provisional Patent Application No. 60/475,633, filed Jun. 4, 2003. The entire contents of these application are incorporated herein by reference in their entirety.

### FIELD OF INVENTION

The present invention relates to methods for treating, killing, and/or inhibiting the growth of Herpes viruses in human subjects comprising topically administering to a human subject in need thereof a nanoemulsion composition having antiviral properties. The present invention also relates to methods for treating and/or preventing lesions associated with Herpes virus infections in human and animal subjects, comprising topically administering to a human or animal subject in need thereof a nanoemulsion composition having antiviral properties.

### BACKGROUND OF THE INVENTION

### A. Herpes Virus Infections

Herpes viruses are a leading cause of human viral disease, second only to influenza and cold viruses. They are capable of causing overt disease or remaining silent for many years only to be reactivated, for example as shingles. The name herpes comes from the Latin herpes which, in turn, comes from the Greek word herpein which means to creep. This reflects the creeping or spreading nature of the skin lesions caused by many herpes virus types.

There are at least 25 viruses in the family Herpesviridae (currently divided into three sub-families; alpha, beta, and gamma). Eight or more herpes virus types are known to infect man, as shown in Table 1.

TABLE 1

| | | |
|---|---|---|
| Human herpes virus 1 | Herpes simplex type 1 (HSV-1) | Alpha |
| Human herpes virus 2 | Herpes simplex type 2 (HSV-2) | Alpha |
| Human herpes virus 3 | Varicella-zoster (VZV) | Alpha |
| Human herpes virus 4 | Epstein-Barr (EBV) | Gamma |
| Human herpes virus 5 | Cytomegalovirus (CMV) | Beta |
| Human herpes virus 6/7 | Exanthum subitum | Beta |
| | Roseola infantum | |
| Human herpes virus 8 | Kaposi's Sarcoma-associated | Gamma |
| | herpes virus (KSHV) | |

Once a patient has become infected by a herpes virus, the infection remains for life. The initial infection may be followed by latency with subsequent reactivation. Herpes viruses infect most of the human population and persons living past middle age usually have antibodies to most of the above herpes viruses with the exception of HHV-8. Herpes viruses are classified by their location in the latent state (Table 2).

TABLE 2
Properties of Herpes viruses

| Human | | | | | |
|---|---|---|---|---|---|
| herpes | | | Target cell | | |
| type | Name | Sub Family | type | Latency | Transmission |
| 1 | HSV-1 | Alphaherpesvirinae | Mucoepithelia | Neuron | Close contact |
| 2 | HSV-2 | Alphaherpesvirinae | Mucoepithelia | Neuron | Close contact |
| | | | | | usually sexual |
| 3 | Varicella Zoster | Alphaherpesvirinae | Mucoepithelia | Neuron | Contact or |
| | virus (VSV) | | | | respiratory |
| | | | | | route |
| 4 | Epstein-Barr | Gammaherpesvirinae | B lymphocyte, | B lymphocytes | Saliva |
| | Virus (EBV) | | epithelia | | |
| 5 | Cytomegalovirus | Betaherpesvirinae | Epithelia, | Monocytes, | Contact, blood |
| | (CMV) | | monocytes, | lymphocytes | transfusions, |
| | | | lymphocytes | and possibly | transplantation, |
| | | | | others | congenital |
| 6 | Herpes | Betaherpesvirinae | T lymphocytes | T lymphocytes | Contact, |
| | lymphotropic | | and others | and others | respiratory |
| | virus | | | | route |
| 7 | Human herpes | Betaherpesvirinae | T lymphocytes | T lymphocytes | Unknown |
| | virus-7 (HHV-7) | | and others | and others | |
| 8 | Human herpes | Gammaherpesvirinae | Endothelial | Unknown | Exchange of |
| | virus-8 (HHV-8) | | cells | | body fluids? |
| | KSHV | | | | |

1. Herpes Simplex Virus 1 and 2

Herpes simplex Virus 1 and 2 are very large viruses with very similar characteristics. Almost any human cell type can be infected by HSV. In many cells, such as endothelial cells and fibroblasts, infection is lytic but neurones normally support a latent infection. The hallmark of herpes infection is the ability to infect epithelial mucosal cells or lymphocytes. A reddened area gives rise to a macule which crusts to form a papule. The fluid in this blister is full of virus. As long as the virus is kept moist it can remain infectious

Herpes simplex 1 and 2 can infect both humans and other animals but only humans show symptoms of disease. HSV-1 and HSV-2 first infect cells of the mucoepithelia or enter through wounds. They then frequently set up latent infections in neuronal cells. The site of the initial infection depends on the way in which the patient acquires the virus. Once epithelial cells are infected, there is replication of the virus around the lesion and entry into the innervating neuron. The virus travels along the neuron to the ganglion. In the case of herpes infections of the oral mucosa, the virus goes to the trigeminal ganglia whereas infections of the genital mucosa lead the virus entering the sacral ganglia. The virus can also travel in the opposite direction to arrive at the mucosa that was initially infected. Vesicles containing infectious virus are formed on the muscosa and the virus spreads. The vesicle heals and there is usually no scar as a result.

Latency:

The virus particles can infect neurons and since only immediate early proteins are made, there is no cytopathic effect (although the presence of the virus can be detected by techniques such as immunofluorescence microscopy using antibodies against the immediate early proteins). Breakage of latency can occur in these cells and the virus travels back down the nerve axon. Lesions now occur at the dermatome, that is the area of skin innervated by a single posterior spinal nerve (including but not limited to the trigeminal nerve). This means that recurrence of infection (and therefore symptoms) occurs at the same site as the initial infection. There are several agents that seem to trigger recurrence, most of which are stress-related. It also appears that exposure to strong sunlight and perhaps fever can lead to recurrence. These factors may cause some degree of immune suppression that leads to renewal of virus proliferation in the nerve cell. Recurrent infections are usually less pronounced than the primary infection and resolve more rapidly.

HSV 1 and 2 infections are life-long and although latency is soon set up, the infected patient can infect others as a result of recurrence. The virus is found in the lesions on the skin but can also be present in a variety of body fluids including saliva and vaginal secretions. Both types of HSV can infect oral or genital mucosa depending on the regions of contact. However, HSV-1 is usually spread mouth to mouth or by transfer of infectious virus to the hands after which the virus may enter the body via any wound or through the eyes. A large proportion of the population has evidence of HSV-1 infection as judged by antibodies. As a result of poor hygiene in underdeveloped countries, HSV-1 antibodies are found in more than 90% of children. HSV-1 can also be transferred by sexual transmission.

HSV-2 is normally spread sexually and is found in the anus, rectum and upper alimentary tract as well as the genital area. In addition, an infant can be infected at birth by a genitally-infected mother. The infant can also be infected in utero if the mother's infection spreads.

Diseases Caused by Herpes Simplex Viruses:

Herpes simplex 1 and 2 can cause severe disease. In each case, the initial lesion looks the same. A clear vesicle containing infectious virus with a base of red (erythomatous) lesion at the base of the vesicle. From this pus-containing (pustular) lesion, encrusted lesions and ulcers may develop. Examples of diseases associated with HSV-1 and HSV-2 infection include oral herpes, herpes keratitis, herpes whitlow, herpes gladiatorum, herpes rugbeiorum, eczema herpeticum, genital herpes, HSV proctitis, HSV Encephalitis, HSV Meningitis, and HSV infection of neonates.

Oral herpesis usually caused by HSV-1, but rarely can be caused by HSV-2. In primary herpetic gingivostomatitis, the typical clear lesions first develop followed by ulcers that have a white appearance. The infection, often initially on the lips can spread to all parts of the mouth and pharynx. Reactivation from the trigeminal ganglia can result in what are known as cold sores. Herpes pharyngitis is often associated with other viral infections of the upper respiratory tract. The disease is more severe in immunosuppressed people such as AIDS patients.

Herpes Labialis (HSV-1), also known as cold sores, is characterized by a high rate of recurrences, most often at the site of initial infection (recurrent Herpes Labialis). The global sero-prevalence of HSV-1 in adults is currently 70-80%, which results in 400 million or more cold sores annually. In the United States 40-50% of the adolescent population and 80-90% of the adult population has been exposed to HSV-1. Approximately 40% of the infected population has had a cold sore at one time or another and most people who have had cold sores will have recurrent outbreaks. Over 50 million adults in the United States have 2 or more outbreaks per year. Episodes generally regress within 7-10 days with complete healing by 12-14 days, although a flat scar or erythema may persist (3). While recurrent Herpes Labialis is a benign disease that regresses spontaneously, it is highly contagious with high viral titers in blisters and effluent. Herpes Labialis causes physical pain and can also be disfiguring especially in those patients with frequent recurrences.

Current treatments for Herpes Labialis can be divided into three major categories: 1) palliative treatment 2) topical antiviral medication 3) systemic antiviral medication. Palliative treatments with numbing agents (lidocaine, tetracaine, benzocaine, benzyl alcohol, camphor, and phenol) and emollients (petrolatum and allantoin) while alleviating some of the discomfort of a recurrence of Herpes Labialis, have no effect on the time course or on the frequency of recurrences. There are several topical and systemic antiviral medications that purport to shorten the time course of Herpes Labialis eruptions. Abreva® (docosanol 10% Cream formula), a topical cream which has been approved by the FDA for over the counter (OTC) salehas no direct anti-viral activity; its proposed mechanism is to prevent viral entry into cells. Abreva® has been shown to shorten mean time to healing by approximately a half-day. For significant response, docosanol must be applied during the prodrome stage. The prescription antiviral drugs used for HSV-1 infections are all analogs of acyclic guanosine: Zovirax® (acyclovir), Valtrex® (valacyclovir), Denavir® (penciclovir), and Famvir® (famciclovir). The FDA has not approved these drugs for OTC sale because of possible development of viral resistance. Due to low bioavailability, Zovirax® has but marginal efficacy and application after the prodromal phase has little or no efficacy. Treatment with penciclovir in 1% concentration (Denavir® 1%) when started during the prodrome is somewhat more effective than Zovirax® in decreasing lesion healing time, alleviation of pain, and viral shedding. However, application after the prodromal phase has but marginal efficacy with 20-30% reduction in symptoms and time to healing. Famvir® (famciclovir) is converted to penciclovir in the body. Famciclovir is active against the same viruses as Acyclovir but has a longer duration of action. Valtrex®, a valine ester of acyclovir, is another “prodrug,” which is converted to acyclovir in the body. Oral Valtrex® (Valacyclovir) is approved for use in immunocompetent adults as a one day treatment. Oral treatment with these acyclovir produgs shortens duration of Herpes Labialis episodes by approximately 1 day. No cure is available for HSV-1 infection, as Herpes lesions are recurrent and life long.

Herpes keratitis is an infection of the eye and is primarily caused by HSV-1. It can be recurrent and may lead to blindness. It is a leading cause of corneal blindness in the United States.

Herpes whitlow is a disease of persons who come in manual contact with herpes-infected body secretions and can be caused by either type of HSV and enters the body via small wounds on the hands or wrists. It can also be caused by transfer of HSV-2 from genitals to the hands.

Herpes gladiatorum is contracted by wrestlers. It apparently spreads by direct contact from skin lesions on one wrestler to his/her opponent, and usually appears in the head and neck region (which are frequently sites of contact in wrestling holds). It is also seen in other contact sports such as rugby where it is known as scrum pox (Herpes Rugbeiorum).

Eczema herpeticum is found in children with active eczema, preexisting atopic dermatitis, and can spread over the skin at the site of eczema lesions. The virus can spread to other organs such as the liver and adrenals.

Genital herpes is usually the result of HSV-2 with about 10% of cases being the result of HSV-1. Recent studies, however, suggest that about one-half of the new cases of genital herpes are caused by HSV-1. Primary infection is often asymptomatic but many painful lesions can develop on the glans or shaft of the penis in men and on the vulva, vagina, cervix and perianal region of women. In both sexes, the urethra can be involved. In women, the infection may be accompanied by vaginal discharge. Genital herpes infections, which involve a transient viremia, can be accompanied by a variety of symptoms including fever, myalgia, and glandular inflammation of the groin area. Secondary episodes of genital herpes, which occur as a result of reactivation of virus in the sacral ganglion, are frequently less severe (and last a shorter time) than the first episode. Recurrent episodes seem usually to result from a primary HSV-2 infection. Patients who are about to experience a recurrence usually first experience a prodrome in which there is a burning sensation in the area that is about to erupt. Some patients have only infrequent recurrences but others experience recurrences as often as every 14-21 days. Whether there is an apparent active disease or not, an infected patient remains infectious without overt symptoms, thus passing the virus to sexual partners unwittingly.

HSV proctitis is an inflammation of the rectum and the anus.

HSV Encephalitis is usually the result of an HSV-1 infection and is the most common sporadic viral encephalitis. HSV encephalitis is a febrile disease and may result in damage to one of the temporal lobes. As a result there is blood in the spinal fluid and the patient experiences neurological symptoms such as seizures. The disease can be fatal but in the US there are fewer than 1000 cases per year.

HSV Meningitis is the result of an HSV-2 infection. The symptoms seem to resolve spontaneously.

HSV infection of neonates results from HSV-2 and is often fatal, although such infections are rare. Infection is especially possible if the mother is shedding virus at the time of delivery. The virus can either be obtained in utero or during birth with the latter being more common. Because the neonate has an underdeveloped immune system, the virus can spread rapidly to many peripheral organs (e.g. lungs and liver) and can infect the central nervous system.

2. Varicella-Zoster Virus

Varicella-Zoster Virus (also known as Herpes Zoster Virus and Human Herpes Virus-3) results in a characteristic rash that forms a belt around the thorax in many patients. This virus causes two major diseases, chicken-pox (Varicella), usually in childhood, and shingles, later in life. Shingles (Zoster) is a reactivation of an earlier varicella infection.

Varicella virus is highly infectious, with more than 90% of the population of the US having antibodies against varicella proteins. In the household of an infected patient, 90% of contacts who have hitherto not had the disease will get it (unless vaccinated). It is spread by respiratory aerosols or direct contact with skin lesions. As with HSV, infection is via mucosa, this time in the respiratory tract.

During the 10-12 day prodromal stage, the virus in the respiratory mucosa infects macrophages and pneumocytes. At this stage, there are no symptoms. The virus spreads from the lungs to lymphocytes and monocytes and to the reticulo-endothelial system. Here, at about 5 days, a second viremia occurs and the virus travels to the skin, mouth, conjunctiva, respiratory tract and, indeed, to epithelial sites throughout the body. The virus then leaves the blood vessels and first infects sub-epithelial sites and then epithelial sites forming papulae containing multinucleated cells with intracellular inclusions. The virus reaches the surface and is shed to the exterior of the respiratory tract about 12-14 days after the initial infection. It takes a little longer (a few days) for the virus to reach the surface of the skin when the characteristic papules (rash) appear. There are various periods between the initial infection and the occurrence of the papules that are diagnostic of chicken pox but the average is about two weeks with range of 10 to 23 days. Spreading of the disease can be from virus in the respiratory tract (by a cough) or from contact with ruptured papulae on the skin containing infectious virus.

The rash is most pronounced on the face, scalp and trunk and less on the limbs. The disease is more severe in older children and adults. This is particularly the case in immunocompromised patients (AIDS, transplantation etc). The spread of the virus may lead to problems in the lungs, liver and to meningitis. In this case mortality may be up to 20%.

Shingles:

After the infectious period, the virus may migrate to the ganglia associated with areas in which the virus is actively replicated. The virus may then be reactivated under stress or with immune suppression. This usually occurs later in life. The recurrence of varicella replication is accompanied by severe radicular pain in discrete areas, those innervated by the nerve in which latent infection has occurred. A few days later chicken pox-like lesions occur in restricted areas (dermatome) that are innervated by a single ganglion. New lesions may appear in adjacent dermatomes and even further afield. Reactivation can affect the eye via the trigeminal nerve (uveitis, keratitis, conjunctivitis, ophthalmoplegia, iritis) and the brain via the cranial nerve VII and VIII (Bell's Palsy and Ramsay-Hunt syndrome). The skin lesions are somewhat different from those in chicken pox, being restricted to small areas of the skin, usually on the thorax. They are small and close together. Reactivation can lead to chronic burning or itching pain called post-herpetic neuralgia which is seen primarily in the elderly. The pain may last well after the rash has healed (even months or years).

3. Epstein-Barr Virus

Epstein-Barr virus is the causative agent of Burkitt's lymphoma in Africa, nasal pharyngeal carcinoma in the orient, and infectious mononucleosis in the west. It was first discovered as the causative agent of Burkitt's lymphoma and it was later found that patients with infectious mononucleosis have antibodies that react with Burkitt's lymphoma cells.

The virus only infects a small number of cell types that express the receptor for complement C3d component (CR2 or CD21). These are certain epithelial cells (oro- and naso-pharynx) and B lymphocytes. This explains the cellular tropism of the virus.

Infectious Mononucleosis:

The primary infection is often asymptomatic but the patient may shed infectious virus for many years. The disease is characterized by malaise, lymphadenopathy, tonsillitis, enlarged spleen and liver and fever. The fever may persist for more than a week. There may also be a rash. The severity of disease often depends on age (with younger patients resolving the disease more quickly) and resolution usually occurs in 1 to 4 weeks. Although infectious mononucleosis is usually benign, there may be complications. These include neurological disorders such as meningitis, encephalitis, myelitis and Guillain-Barrè syndrome.

4. Cytomegalovirus

Cytomegalovirus infection is found in a significant proportion of the population. As with Epstein-Barr virus, seropositivity increases with age. By college age, about 15% of the US population is infected and this rises to about 50% by 35 years of age.

Cytomegalovirus causes no symptoms in children and for most adults the disease is mild. In patients who have received an organ transplant or have an immunosuppressive disease (e.g. AIDS), cytomegalovirus can be a major problem. Particularly important is cytomegalovirus-retinitis in the eye which occurs in up to 15% of all AIDS patients.

5. Other Herpes Viruses

Human herpes virus 6 is found worldwide and is found in the saliva of the majority of adults (>90%). It infects almost all children by the age of two and the infection is life-long. It replicates in B and T lymphocytes, megakaryocytes, glioblastoma cell and in the oropharynx. It can set up a latent infection in T cells which can later be activated when the cells are stimulated to divide. Cell-mediated immunity is essential in control, although infection is life-long, and the virus can reactivate in immune-suppression.

Human herpes virus-6 has two forms, HHV-6A and HHV-6B. The latter causes exanthem subitum, otherwise known as roseola infantum. This a common disease of young children (in the US>45% of children are seropositive for HHV-6 by two years of age) and symptoms include fever and sometimes upper respiratory tract infection and lymphadenopathy. The symptoms last a few days after an incubation period of around 14 days. The fever subsides leaving a macropapular rash on the trunk and neck that lasts a few days longer. In adults, primary infection is associated with a mononucleosis. This virus was originally isolated from patients with a lymphoproliferative disease and may co-infect HIV-infected T4 lymphocytes exacerbating the replication of HIV. Patients with HIV have a higher infection rate than the normal population.

Human herpes virus 7 binds to the CD4 antigen and replicates in T4 (CD4+) cells and is found in the saliva of the majority of the adult population (>75%). Most people acquire the infection as children and it remains with them for the rest of their lives. It is similar to HHV-6 and may be responsible for some cases of exanthem subitum.

Human herpes virus 8 was formerly known as Kaposi's sarcoma associated herpes virus and is found in the saliva of many AIDS patients. It infects peripheral blood lymphocytes.

Finally, Herpes B virus is a simian virus found in old world monkeys such as macaques but it can be a human pathogen in people who handle monkeys (monkey bites are the route of transmission). In humans, the disease is much more problematic than it is in its natural host. Indeed, about 75% of human cases result in death with serious neurological problems (encephalitis) in many survivors. There is also evidence that the disease can be passed from a monkey-infected human to another human.

### B. Conventional Treatment Options for Herpes Virus Infections

There are a variety of nucleoside analog drugs used to treat herpes infections such as HSV-1, HSV-2, and Varicella. Examples of nucleoside analogs used to treat herpes infections include acyclovir, famciclovir, and valacyclovir. All of these nucleoside analogs suffer from the appearance of resistant herpes mutants. In addition, these drugs act against the replicating virus and therefore they are ineffective against latent virus.

Specifically, the US Food and Drug Administration has stated that “[t]he emergence of herpesvirus (HSV) isolates that are resistant to each of the marketed acyclic guanosine analogues has been documented. It is generally believed that the development of resistance is more commonly associated with HSV-2 than HSV-1 and that a higher frequency of HSV resistance, overall, occurs among immunocompromised individuals than among those with intact immune systems. Because of a common mechanism of action, it is also generally believed that the rate of cross-resistance between available acyclic guanosine analogues is nearly complete. Thus, the Agency is concerned that misuse of these drugs could hasten the development of HSV resistance, jeopardizing the usefulness of the entire class of agents for treatment of serious and life-threatening herpes infections. This concern is further enhanced by the fact that currently no other classes of agents are available that have safety and efficacy comparable to the acyclic guanosine analogues in the treatment for these infections. These concerns reflect a long-term public health issue with broader implications than safety and tolerability in an individual patient.” Food and Drug Administration, Center for Drug Evaluation and Research, March 2000

Acyclovir (Zovirax®) is a synthetic purine nucleoside analogue active against herpes simplex virus types 1 (HSV-1), 2 (HSV-2), and varicella-zoster virus (VZV). Zovirax Capsules, Tablets, and Suspension are formulations for oral administration. Resistance of HSV and VZV to acyclovir can result from qualitative and quantitative changes in the viral TK and/or DNA polymerase. Clinical isolates of HSV and VZV with reduced susceptibility to acyclovir have been recovered from immunocompromised patients, especially with advanced HIV infection. Adverse effects or events associated with acyclovir include anaphylaxis, angiodema, fever, headache, pain, peripheral edema, aggressive behavior, agitation, ataxia, coma, confusion, decreased consciousness, delirium, dizziness, dysarthria, encephalopathy, hallucinations, paresthesia, psychosis, seizure, somnolence, tremors, diarrhea, gastrointestinal distress, nausea, anemia, leukocytoclastic, vasculitis, leukopenia, lymphadenopathy, thrombocytopenia, hepatitis, hyperbilirubinemia, jaundice, myalgia, alopecia, erythema multiforme, photosensitive rash, pruritis, rash, Stevens-Johnson syndrome, toxic epidermal necrolysis, urticaria, renal failure, elevated blood urea nitrogen, elevated creatinine, hematuria, and visual abnormalities.

Famciclovir (Famvir®) is an orally administered tablet used to treat herpes zoster (shingles; a rash that can occur in people who have had chickenpox in the past). It is also used to treat repeat outbreaks of herpes virus cold sores or fever blisters in people with a normal immune system. Famciclovir is used to treat repeat outbreaks and to prevent further outbreaks of genital herpes (a herpes virus infection that causes sores to form around the genitals and rectum from time to time) in people with a normal immune system. Famciclovir is also used to treat returning herpes simplex infections of the skin and mucous membranes (mouth, anus) in people with human immunodeficiency virus (HIV) infection.

Famciclovir is in a class of medications called antivirals. It works by stopping the spread of the herpes virus in the body. Famciclovir does not cure herpes infections and may not stop the spread of herpes virus to other people. However, it may decrease the symptoms of pain, burning, tingling, tenderness, and itching and help sores to heal Side effects associated with famciclovir include headache, nausea, vomiting, diarrhea or loose stools, gas, stomach pain, tiredness, rash, itching, painful menstrual periods, and pain, burning, numbness, or tingling in the hands or feet.

Valacyclovir (Valtrex®) is an orally administered drug used to treat herpes zoster (shingles) and genital herpes. It does not cure herpes infections but decreases pain and itching, helps sores to heal, and prevents new ones from forming. Side effects associated with valacyclovir include headache, upset stomach, vomiting, diarrhea or loose stools, constipation, rash, itching, confusion, yellowness of the skin or eyes, fever, and blood in the urine.

The antiviral medications available in oral form (acyclovir, valacyclovir, famciclovir) have been specifically developed for the treatment of genital herpes, although they can be prescribed for oral herpes. One problem with the use of systemic prescription products for treating herpes lesions is that the drugs are not readily accessible to patients in a timely manner, as treatment should begin within 1-4 hours of the onset of symptoms.

There are two topical antiviral medications prescribed for the treatment of oral HSV symptoms: acyclovir ointment (brand name Zovirax®) and penciclovir cream (brand name Denavir®). Both work to speed up the healing process and reduce the viral activity; however, the drugs only provide palliative relief or shorten outbreaks only by about 12 hours. These topical drugs are put directly on the lesions themselves, but can also be used at the onset of prodrome.

Other topical treatments for oral herpes are available over-the-counter (OTC), but are not antiviral compounds like acyclovir and penciclovir. Some also contain ingredients that numb the area and induce temporary relief from the discomfort of an outbreak. Unfortunately, some OTC treatments may actually delay the healing time of symptoms because they can further irritate the area with repeated applications. There is only one OTC FDA-approved cream, Abreva®, which has been clinically proven to help speed the healing process.

Unlike herpes simplex virus, there are no drugs available to treat Epstein-Barr virus. This may reflect the absence of a thymidine kinase encoded by this virus (drugs such as acyclovir that are active against herpes simplex are activated by the viral thymidine kinase).

For cytomegalovirus (CMV) treatment, ganciclovir, which inhibits the replication of all human herpes viruses, is usually used, especially to treat retinitis. Foscarnet is also approved in the US. Acyclovir is not effective.

Ganciclovir is an orally administered drug used to treat cytomegalovirus (CMV) retinitis (eye infection that can cause blindness) in people whose immune system is not working normally. Ganciclovir capsules are used to treat CMV retinitis after the condition has been controlled by intravenous ganciclovir. Ganciclovir is also used to prevent cytomegalovirus (CMV) disease in people who have acquired immunodeficiency syndrome (AIDS) or who have received an organ transplant and are at risk of CMV disease. Ganciclovir can have serious side effects, including upset stomach, vomiting, diarrhea, constipation, stomach pain, belching, loss of appetite, changes in ability to taste food, dry mouth, mouth sores, unusual dreams, nervousness, depression, sweating, flushing, joint or muscle pain or cramps, seeing specks, flashes of light, or a dark curtain over everything, decreased urination, hives, rash, itching, swelling of the hands, arms, feet, ankles, or lower legs, numbness, pain, burning, or tingling in the hands or feet, shaking hands that you cannot control, difficulty breathing or swallowing, chest pain, mood changes, and seizures. In addition, ganciclovir may lower the number of all types of cells in blood, causing serious and life-threatening problems. Moreover, laboratory animals who were given ganciclovir developed birth defects, a lower sperm count, and cancer. It is not known if ganciclovir causes birth defects, lower sperm count or fertility problems, or cancer in people.

The recommended treatments for Herpes B virus are Acyclovir and Ganciclovir, although their efficacy is unknown

### C. Nanoemulsions

Prior teachings related to nanoemulsions are described in U.S. Pat. No. 6,015,832, which is directed to methods of inactivating a Gram positive bacteria, a bacterial spore, or a Gram negative bacteria. The methods comprise contacting the Gram positive bacteria, bacterial spore, or gram negative bacteria with a bacteria-inactivating (or bacterial-spore inactivating) emulsion. U.S. Pat. No. 6,506,803 is directed to methods of killing or neutralizing microbial agents (e.g., bacterial, virus, spores, fungus) on or in humans using an emulsion. U.S. Pat. No. 6,559,189 is directed to methods for decontaminating a sample (human, animal, food, medical device, etc.) comprising contacting the sample with a nanoemulsion. The nanoemulsion, when contacted with bacterial, virus, fungi, or spores, kills or disables the pathogens. The antimicrobial nanoemulsion comprises a quaternary ammonium compound, one of ethanol/glycerol/PEG, and a surfactant. U.S. Pat. No. 6,635,676 is directed to two different compositions and methods of decontaminating samples by treating a sample with either of the compositions. Composition 1 comprises an emulsion that is antimicrobial against bacterial, virus, fungi, and spores. The emulsions comprise an oil and a quaternary ammonium compound. U.S. Pat. No. 7,314,624 is directed to methods of inducing an immune response to an immunogen comprising treating a subject via a mucosal surface with a combination of an immunogen and a nanoemulsion. The nanoemulsion comprises oil, ethanol, a surfactant, a quaternary ammonium compound, and distilled water. US-2005-0208083-A1 and US-2006-0251684-A1 are directed to nanoemulsions having droplets with preferred sizes. US-2007-0054834-A1 is directed to compositions comprising quaternary ammonium halides and methods of using the same to treat infectious conditions. The quaternary ammonium compound may be provided as part of an emulsion. Finally, US-2007-0036831-A1 is directed to nanoemulsions comprising an anti-inflammatory agent.

There is a need in the art for improved treatment options for patients affected by herpes infections. Specifically, there is a need in the art for highly effective topical agents that can reduce the healing time required for lesions associated with herpes infection. The present invention satisfies this need.

### SUMMARY OF THE INVENTION

The present invention is directed to a method of treating a herpes virus infection, preventing a herpes virus infection, preventing recurrent herpes virus infection, preventing reactivation of a herpes virus, minimizing reactivation of a herpes virus, or a combination thereof, in a human subject in need thereof. The method comprises topically or intradermally administering to the human subject a nanoemulsion, wherein the topical application is to the herpes lesion, the skin surrounding the herpes lesion, or a combination thereof. The nanoemulsion comprises droplets having an average diameter of less than about 1000 nm, and the nanoemulsion comprises water, at least one oil, at least one surfactant, and at least one organic solvent. In a further embodiment, the nanoemulsion kills the herpes virus and prevents the spread of the virus.

In one embodiment of the invention, the method of the invention comprising applying a nanoemulsion according to the invention to a subject in need results in a reduced time to heal as compared to vehicle, no treatment, or a non-nanoemulsion method of treatment.

In one embodiment of the invention, the surfactant present in the nanoemulsion is a cationic surfactant. In another embodiment of the invention, the nanoemulsion further comprises a chelating agent.

The nanoemulsion in and of itself has anti-viral activity and does not need to be combined with another active agent to obtain therapeutic effectiveness.

In another embodiment, the nanoemulsion further comprises one or more active agents useful in treating, healing or palliating a herpes infection, including but not limited to the addition of another antiviral agent.

Surprisingly, it was discovered that the topically applied nanoemulsions are as effective or better, than conventional topical treatments and orally administered antiviral treatments for Herpes virus infections. This is significant, as a topically applied drug, and therefore local, site-specific activity, is highly preferable over an orally administered drug having systemic activity. As noted in the background section, systemic antiviral drugs have many side effects, some very serious.

In one embodiment of the invention, the nanoemulsions of the invention are (a) therapeutically effective against the herpes virus, and/or (b) viricidal or viristatic against the herpes virus.

In another embodiment of the invention, following treatment with a nanoemulsion according to the invention, partial or complete clearing of lesions is observed. The nanoemulsions of the invention can prevent lesions from appearing or developing. The nanoemulsions of the invention can also reduce time to healing as compared to a control and/or as compared to conventional, non-nanoemulsion treatments such as Abreva®, Zovirax®, and Denavir®. For example, nanoemulsions of the invention can reduce the time to healing when the baseline is the prodrome lesion stage, when the baseline is the erythema lesion stage, when the baseline is the papule lesion stage, and/or when the baseline is the vesicle lesion stage.

The patient to be treated may suffer from a Herpes virus infection, such as an infection by Herpes Simplex Virus Type 1 (HSV-1), Herpes Simplex Virus Type 2 (HSV-2), Varicella Zoster Virus (VZV), Epstein-Bar Virus (EBV), Cytomegalovirus (CMV), Herpes Lymphotropic Virus, Human Herpes Virus Type 7 (HHV-7), Human Herpes Virus Type 8 (HHV-8), or a combination thereof.

The nanoemulsion can be applied to any bodily region needing treatment, including for example the oralfacial region, the eye, the uro-genital region (external or internal, skin or mucosa), vaginal mucosa, rectal mucosa, anal mucosa, oral mucosa, extremities, skin, oral pharynx, superficial skin structure and appendages, lips, vermillion border, all areas of the mouth neck, perineum, upper legs, hand, cornea, eye, urethra, or any combination thereof.

Preferably, the nanoemulsions for topical or intradermal administration are in the form of ointments, creams, emulsions, lotions, gels, liquids, bioadhesive gels, sprays, shampoos, aerosols, pastes, foams, sunscreens, capsules, microcapsules, or in the form of an article or carrier, such as a bandage, insert, syringe-like applicator, pessary, powder, talc or other solid, shampoo, cleanser (leave on and wash off product), and agents that favor penetration within the epidermis, the dermis and keratin layers. The nanoemulsions of the invention can be viricidal or viristatic.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### DESCRIPTION OF THE DRAWINGS

FIG. 1 shows the proposed mechanism of action of a nanoemulsion according to the invention (“NB-001”) when the nanoemulsion is applied to a Herpes lesion resulting from Herpes labialis. It is thought that the nanoemulsion lyses the virus, resulting in deactivation of the virus, death of the virus, or a combination thereof.

FIG. 2 shows a mouse model of HSV-1 infection where a nanoemulsion according to the invention (NB-001) prevented lesion formation and improved the survival of mice infected with a lethal dose of HSV-1. The results show that topical nanoemulsions prevent systemic viral infection in mice to a similar extent as systemic acyclovir.

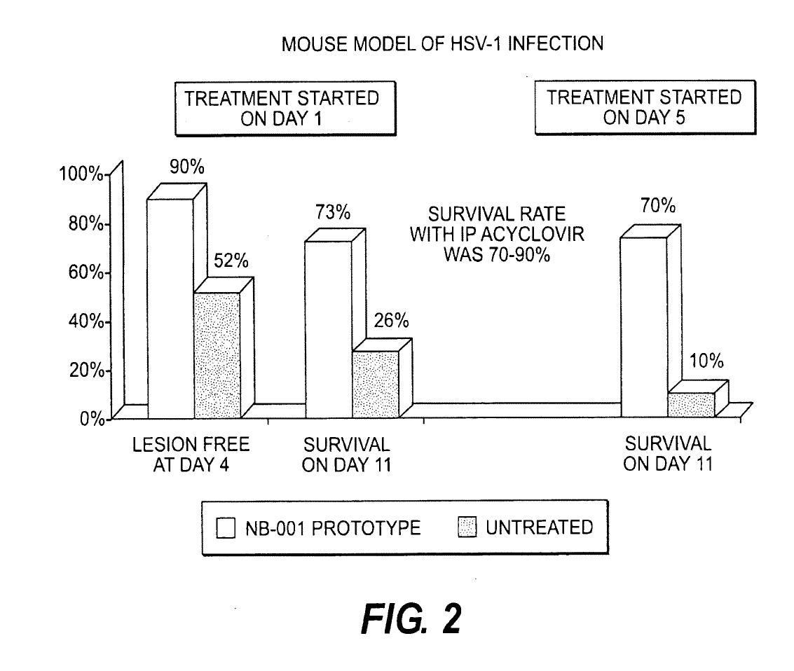

FIG. 3 shows the results of a clinical study of the use of a nanoemulsion according to the invention (NB-001) in treating Herpes Labialis, where NB-001 improved healing of herpes lesions as compared to vehicle. FIG. 3A shows the percentage of subjects healed by day 3 when treated with vehicle, 0.05% nanoemulsion, or 0.10% nanoemulsion, and FIG. 3B shows the percentage of subjects healed by day 4 when treated with vehicle, 0.05% nanoemulsion, or 0.10% nanoemulsion.

FIG. 4 shows the disposition and demographics of subjects in a Phase 2B Herpes Labialis trial when treated with vehicle and nanoemulsions according to the invention (0.1% NB-001, 0.3% NB-001, and 0.5% NB-001).

FIG. 5 shows a summary of adverse events in a Phase 2B Herpes Labialis Trial using a nanoemulsion according to the invention (NB-001), where the incidence of adverse events is not different between treated subjects treated with a nanoemulsion and subjects treated with vehicle.

FIG. 6 shows the time to healing in days (Kaplan-Meier Life Table Analysis (ITT)) assessed by subjects in a Phase 2B trial of treating Herpes Labialis using a nanoemulsion according to the invention. The primary analysis indicates significant improvement in time to healing, particularly for subjects treated with 0.3% nanoemulsion.

FIG. 7 shows the time to healing in days (Kaplan-Meier Life Table Analysis (ITT)) assessed by the investigators in a Phase 2B trial of treating Herpes Labialis using a nanoemulsion according to the invention. The primary analysis indicates significant improvement in time to healing, particularly for subjects treated with 0.3% nanoemulsion.

FIG. 8 shows the improvement in time to healing demonstrated in the Phase 2B trial of treating Herpes Labialis using a nanoemulsion according to the invention (final analysis for the 0.30% nanoemulsion).

FIG. 9 shows a comparison of the efficacy levels reported separately for leading marketed therapies as compared to a nanoemulsion according to the invention in treating Herpes Labialis. Reported efficacy values are shown for oral Famvir®, oral Valtrex®, topical Abreva®, topical Zovirax®, and topical Denavir®.

FIG. 10 are electron micrographs showing lysing of HSV-1 by a nanoemulsion according to the invention (NB-001). FIG. 10A shows HSV-1 virus prior to application of NB-001; FIG. 10B shows HSV-1 virus 15 minutes after application of NB-001, showing NB-001 surrounding and fusing with HSV-1 viruses; and FIG. 10C shows HSV-1 virus 30 minutes after application of NB-001, showing NB-001 disrupting and lysing HSV-1 organisms.

FIG. 11 shows the inhibition of various strains of HSV-1 by a nanoemulsion according to the invention (NB-001). The HSV-1 strains tested were wild type (WT), resistant to the nucleoside analogue acyclovir (ACV-R), resistant to the pyrophosphate analogue foscarnet (FOS-R), or resistant to both acyclovir and foscarnet (ACV/FOS-R). The data show that nanoemulsions according to the invention are active against both acyclovir and foscarnet resistant HSV-1 strains.

FIG. 12 shows the inhibition of various strains HSV-2 by a nanoemulsion according to the invention (NB-001). The HSV-2 strains tested were wild type (WT), resistant to the acyclovir (ACV-R), resistant to the foscarnet (FOS-R), or resistant to both acyclovir and foscarnet (ACV/FOS-R). The data show that nanoemulsions according to the invention are active against both acyclovir and foscarnet resistant HSV-2 strains.

FIG. 13 shows the effect of various nanoemulsion doses on delivery into pig skin at 24 hours (0.1%, 0.3%, and 0.5% nanoemulsion, corresponding to 0.1%, 0.3%, and 0.5% CPC), with nanoemulsions according to the invention containing differing amounts of a surfactant, CPC. FIG. 13A shows the results of epidermal delivery and FIG. 13B shows the results of dermal delivery.

FIG. 14 shows the effect of higher nanoemulsion doses on the permeation into the epidermis of human cadaver skin (0.1%, 0.3%, and 0.5% nanoemulsion, corresponding to 0.1%, 0.3%, and 0.5% CPC), 24 hours following 5 applications within 12 hours. Cross polar light microscopy also demonstrates crystallization of CPC from the nanoemulsion when applied to skin at higher concentrations.

FIG. 15 shows the effect of higher nanoemulsion doses on the permeation into the dermis of human cadaver skin (0.1%, 0.3%, and 0.5% nanoemulsion, corresponding to 0.1%, 0.3%, and 0.5% CPC), 24 hours following 5 applications within 12 hours.

FIG. 16 illustrates the dimensions of a lateral diffusion study utilizing human cadaver skin described in Example 7, with two concentric glass rings defining an outer dosing area of 5.27 cm², a middle area of 3.3 cm², and an inner area of 0.5 cm².

FIG. 17 illustrates the design of a lateral diffusion study described in Example 7.

FIG. 18 graphically describes the results of a lateral diffusion study utilizing human cadaver skin and a nanoemulsion according to the invention comprising 0.5% cetylpyridinium chloride (CPC) as compared to a control composition comprising 0.5% cetylpyridinium chloride (CPC) aqueous solution. The results of lateral diffusion over a 24 hour period are depicted, with minimal lateral diffusion into the middle region and no lateral diffusion shown in the inner region for the aqueous CPC solution composition. In contrast, lateral diffusion was clearly measured for the middle and inner regions when the 0.5% nanoemulsion was applied.

FIG. 19 graphically shows the results of the lateral diffusion study described in Example 7, wherein the transport of a nanoemulsion according to the invention, NB-002 (0.5% NB-002 and 0.25% NB-002) within epidermal tissue is exhibited in all three regions: the outer dosing region and the middle and inner regions.

FIG. 20 graphically shows the results of the lateral diffusion study described in Example 7, wherein the transport of 0.5% NB-002 and 0.25% NB-002 within dermal tissue is exhibited in all three regions: the outer dosing region and the middle and inner regions.

FIG. 21 graphically shows the lateral diffusion of the tested 0.5% NB-002 within the epidermis 24 hours after a single application in the outer dosing region, with measurable amounts of nanoemulsion detected in the outer, middle, and inner regions.

FIG. 22 graphically shows the lateral diffusion of NB-002 within the dermis 24 hours after a single application in the outer dosing region, with measurable amounts of nanoemulsion detected in the outer, middle, and inner regions.

FIG. 23 graphically shows the lateral diffusion of NB-002 within the epidermis 24 hours after an application in the outer dosing region at time 0 and 8 hours, with measurable amounts of nanoemulsion detected in the outer, middle, and inner regions that exceeded the minimum fungicidal concentration (MFC₉₀) of 4 μg/g NB-002, as measured by CPC which is used as a marker for NB-002.

FIG. 24 graphically shows the lateral diffusion of NB-002 within the dermis 24 hours after an application in the outer dosing region at time 0 and 8 hours, with measurable amounts of nanoemulsion detected in the outer, middle, and inner regions that exceeded the, minimum fungicidal concentration (MFC₉₀) of more than 4 μg/g as measured by CPC which is used as a marker for NB-002.

FIG. 25 shows the absorption of two different nanoemulsion formulations comprising terbinafine hydrochloride (TB) into the epidermis (dorsal skin) of pig skin in comparison to Lamisil® cream, both shown at 24 hours after two dosings (0 and 8 hours) with a nanoemulsion or Lamisil®.

FIG. 26 shows the absorption of two different nanoemulsion formulations comprising terbinafine hydrochloride (TB) into the dermis of pig skin in comparison to Lamisil® cream, both shown at 24 hours after two dosings (0 and 8 hours) with a nanoemulsion or Lamisil®.

FIG. 27 shows the viricidal activity, in vitro, of a nanoemulsion according to the invention against the herpes virus HSV-1 KOS. FIG. 27A shows the reduction of HSV-1 by a nanoemulsion according to the invention (NB-001) 15 minutes following application, and FIG. 27B shows the reduction of HSV-1 by NB-001 at its IC₅₀.

FIG. 28 shows levels of miconazole (MCZ) in swine skin epidermis at 24 hours after topical application (BID dosing) for MCZ incorporated into a nanoemulsion (NB-002) as compared to MCZ topically applied in a non-nanoemulsion formulation (Lotramin® AF Spray Solution), demonstrating the significantly improved delivery of the MCZ into the epidermis when MCZ is incorporated into a nanoemulsion.

FIG. 29 shows levels of miconazole (MCZ) in swine skin dermis at 24 hours after topical application (BID dosing) for MCZ incorporated into a nanoemulsion (NB-002) as compared to MCZ topically applied in a non-nanoemulsion formulation (Lotramin® AF Spray Solution), demonstrating the significantly improved delivery of the MCZ into the dermis when MCZ is incorporated into a nanoemulsion.

FIG. 30 shows that the Herpes viruses are enveloped viruses. The viral membrane is quite fragile and a virus with a damaged envelope is not infectious. This means that the virus readily falls apart and so the virus can only be obtained by direct contact with mucosal surfaces or secretions of an infected person.

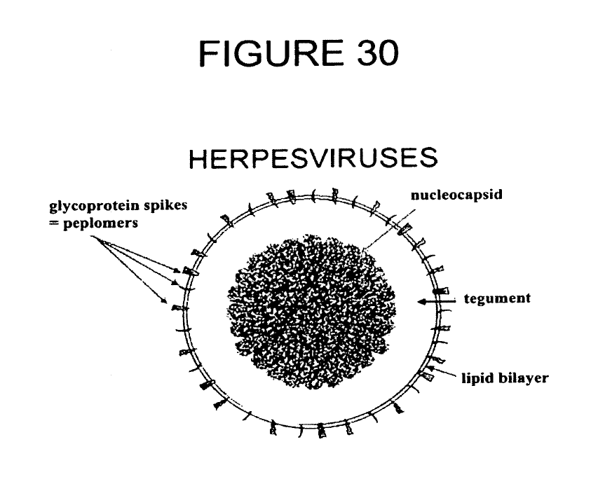

### DETAILED DESCRIPTION OF THE INVENTION

The present invention is directed to a method of treating a herpes virus infection, preventing a herpes virus infection, preventing recurrent herpes virus infection, preventing reactivation of a herpes virus, minimizing reactivation of a herpes virus, or a combination thereof, in a human subject in need thereof comprising topically or intradermally administering to the human or animal subject a nanoemulsion. The topical application is to the herpes lesion, the skin surrounding the herpes lesion, or a combination thereof. The nanoemulsion comprises droplets having an average diameter of less than about 1000 nm, and the nanoemulsion comprises water, at least one oil, at least one surfactant, and at least one organic solvent. The nanoemulsion can further comprise a chelating agent. In one embodiment of the invention, the nanoemulsion kills the herpes virus. The Herpes virus infection to be treated can be latent, active, or reactivated.

One of the problems with conventional drugs used for treating lesions resulting from herpes virus infections is that topically applied conventional treatments have minimal effectiveness. Orally administered drugs may address this problem present in topically applied therapies, but orally administered drugs act systemically and, therefore, may cause hepatoxicity and other side effects discussed in the background of the invention.

Surprisingly, it was discovered that the topically applied nanoemulsions of the invention are as effective, or better, in treating lesions resulting from Herpes virus infections as compared to orally administered conventional treatments for herpes virus infections. This is significant, as a topically applied, and therefore local, site specific activity, is highly preferable over an orally administered, and therefore systemic activity. The nanoemulsions of the invention have equivalent or better efficacy in treating lesions associated with Herpes virus infections as compared to orally administered drugs and commercially available topically applied antiviral drugs.

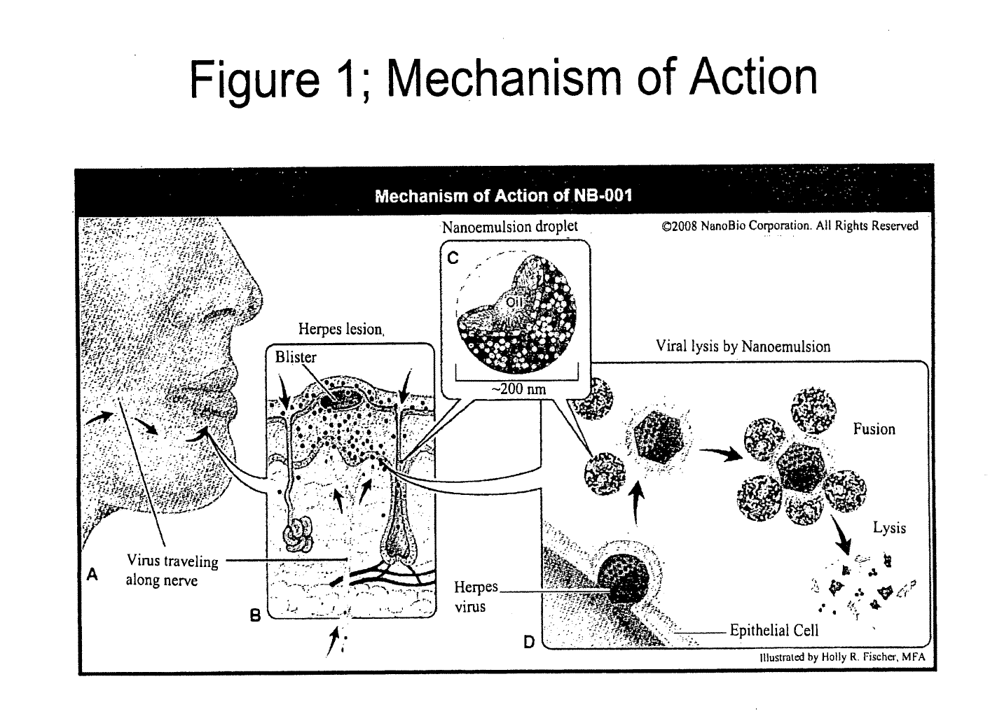

The proposed mechanism of action of the nanoemulsions of the invention is depicted in FIG. 1. The nanoemulsion droplets, having an average diameter of less than about 1000 nm, can be topically applied to the skin, or injected between the skin layers (intradermally). The nanoemulsion droplets migrate through the skin pores/superficial skin structures to reach the site of Herpes virus infection. While the inventors do not wish to be bound by theory, it is thought that the nanoemulsion droplets fuse with the lipids in the viral envelope causing membrane disruption and lysis of the Herpes viruses, thereby “killing” the virus on contact.

Partial or complete clearing of lesions resulting from Herpes virus infection can be obtained using the nanoemulsions and methods of the invention.

### Time to Heal

Surprisingly, it was discovered that the nanoemulsions of the invention can reduce the time to healing, as compared to a control, as measured using a Kaplan-Meier analysis. For example, following treatment the mean or median time to healing of lesions, as compared to a control, can be decreased by at least 12 hours, at least 1 day (24 hour period), at least 36 hours (1.5 days), at least 2 days (48 hours), at least 3 days, at least 3.5 days, at least 4 days, at least 4.5 days, or at least 5 days. See the “time to heal” data presented in the Examples.

For example, the Kaplan-Meier survival curve of investigator-assessed time to healing provided in Example 2 demonstrated a trend toward reduced healing times in all active treatment groups as compared to a vehicle group. In a 0.3% NB-001 group, there was a statistically significant shortening in median and mean time to healing of 1.0 days and 1.3 days, respectively, as compared to the vehicle group.

How “time to healing” is measured can significantly affect the end results. For example, several studies of herpes labialis lesions have excluded subjects who have a lesion at baseline. (Spruance et al., “Single-dose, patient-initiated famciclovir: A randomized, double-blind, placebo-controlled trial for episodic treatment of herpes labialis”, J. Am. Acad. Dermatol., 55:47-53 (2006); Spruance et al., “High-dose, short-duration, Early valacyclovir therapy for episodic treatment of cold sores: results of two randomized, placebo-controlled, multicenter studies,” Antimicrob. Agents Chemother., 47(3):1072-1080 (2003).) However, many cold sore sufferers will have a lesion at the time of needing treatment, either because they do not have prodromal symptoms or they cannot start treatment prior to eruption of a lesion. This may represent at least half of the total population of cold sore sufferers. For example, about 75% of subjects in the Herpes Labialis study using a nanoemulsion according to the invention, described in Example 5 below, already had a lesion by the time of the first investigator assessment and would have been excluded from other cold sore studies. Excluding these subjects over estimates the benefit of these other products in the general population of cold sore sufferers. WHen these subjects are included, the treatment effect with other products is significantly reduced or non existent. In particular, a study of docosanol (Abreva®) published by Sacks only allowed enrollment of subjects who did not have a blister at baseline. (Sacks et al., “Clinical efficacy of topical docosanol 10% cream for herpes simplex labialis: A multicenter, randomized, placebo-controlled trial,” J. Am. Acad. Dermatol., 45:222-230 (2001).) In the Sacks' docosanol study, subjects who demonstrated the onset of cold sore symptoms (prodrome) were to report to the clinic and were only enrolled if they did not show evidence of a lesion. In contrast, the study described in Example 5 below allowed all subjects regardless of stage at baseline.

As described in Example 2 below, treatment with nanoemulsions according to the invention resulted in a 1.7 day improvement over vehicle in subjects who did not have a lesion at baseline, as compared to 0.5-day reduction in the time to healing for subjects treated with docosanol (Abreva®), is currently the most widely used treatment for recurrent labialis. Thus, the data described in Example 2 below suggests that starting treatment prior to the onset of a lesion, i.e., during the prodrome or erythema stage, resulted in an even greater treatment effect with a nanoemulsion according to the invention. A reduction in time to healing of recurrent facial lesions of one day or more is a highly desirable property.

Thus, in one embodiment of the invention, following treatment with a nanoemulsion according to the invention, partial or complete clearing of lesions is observed. The nanoemulsions of the invention can prevent lesions from appearing or developing. The nanoemulsions of the invention can also reduce time to healing as compared to a control and/or as compared to conventional, non-nanoemulsion treatments such as Abreva®. For example, nanoemulsions of the invention can reduce the time to healing when the baseline is the prodrome lesion stage, when the baseline is the erythema lesion stage, when the baseline is the papule lesion stage, and/or when the baseline is the vesicle lesion stage.

### Additional Embodiments

Further, it was also discovered that following treatment the incidence of aborted lesions can be increased, as compared to a control. See e.g., Table 3 below.

TABLE 3
Percent Aborted Lesions in Subjects Assessed
as Prodrome or Erythema Stage at Baseline

| Vehicle | 0.1% NB-001 | 0.3% NB-001 | 0.5% NB-001 |
|---|---|---|---|
| (N = 28) | (N = 31) | (N = 34) | (N = 28) |
| 21.4% | 19.4% | 35.3% | 17.9% |

This means that in one embodiment of the invention, the methods encompass prevention of lesions, as well as shortened time to heal for lesions. In addition, after treatment with a nanoemulsion according to the invention, the subject may not shed virus for as long of a time period. This is significant, as viral shedding results in spreading of the HSV-1 virus. Elimination of viral shedding or reducing the time of viral shedding eliminates or minimizes contraction of HSV-1 by others associated with exposure to the HSV-1 infected individual. Notably, there is no published data demonstrating that Abreva® has an effect on viral shedding.

The Herpes virus can be, for example, Herpes Simplex Virus Type 1 (HSV-1), Herpes Simplex Virus Type 2 (HSV-2), Varicella Zoster Virus (VZV), Epstein-Bar Virus (EBV), Cytomegalovirus (CMV), Herpes Lymphotropic Virus, Human Herpes Virus Type 7 (HHV-7), Human Herpes Virus Type 8 (HHV-8), or a combination thereof.

In one embodiment of the invention, the nanoemulsion is applied to the oralfacial region, the eye, the uro-genital region (external or internal, skin or mucosa), vaginal mucosa, rectal mucosa, anal mucosa, oral mucosa, extremities, skin, oral pharynx, superficial skin structure and appendages, lips, vermillion border, all areas of the mouth, neck, perineum, upper legs, hand, cornea, eye, urethra, or any combination thereof.

In another embodiment of the invention, the method is used to treat a subject having resistance to one or more antiviral agents, such as resistance to nucleoside analogs, e.g., acyclovir. In contrast to traditional antiviral drugs, such as acyclovir, subjects do not develop resistance to treatment by a nanoemulsion according to the invention. This is because the physical mechanism of action of a nanoemulsion according to the invention renders the emergence of drug resistance to the nanoemulsion improbable. Repeated passages in vitro in the presence of sub-lethal concentrations of a nanoemulsion according to the invention (NB-001) have not produced any stable HSV-1 resistant strains. In addition, no cross-resistance has been observed with existing antiviral agents. This is significant, as almost all anti-microbials, including anti-virals, are subject to drug resistance as the pathogens mutate over time, becoming less susceptible to the treatment.

The method of the invention is applicable to preventing lesions. In such a method, the Herpes virus is latent. Herpes viruses may be latent, for example, in the trigeminal ganglion, B lymphocyte, lumbrosacral ganglia, monocytes, neuron, T lymphocyte, or epithelial cells. Thus, in one embodiment of the invention, the nanoemulsion is preventative against the herpes infection, recurrent infection, or reactivation of virus.

Examples of Herpes virus infections that can be treated using the methods of the invention include, but are not limited to, herpes labialis, genital herpes, ocular herpes, herpes rugbiorum, herpes gladiatorum, or herpetic whitlow.

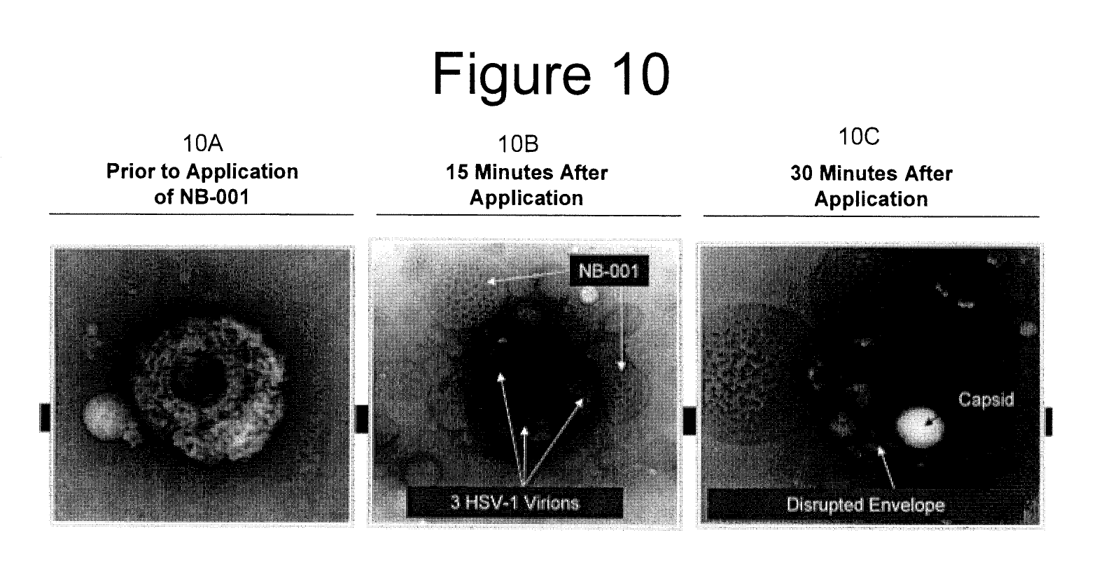

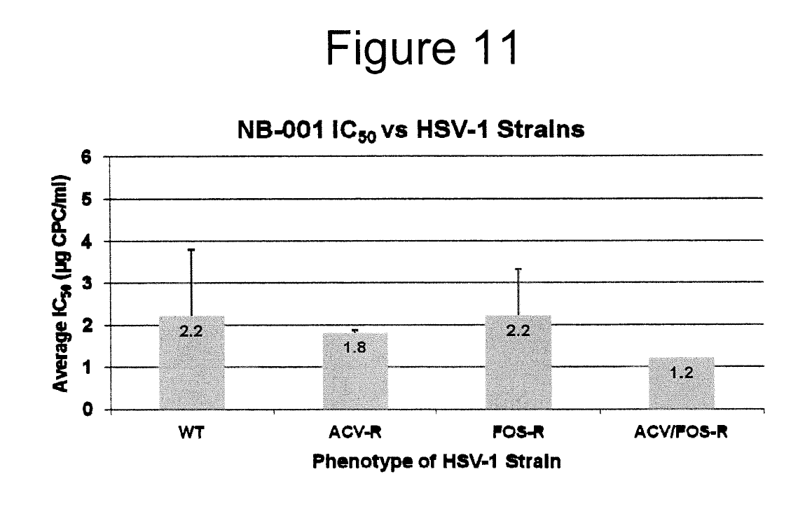

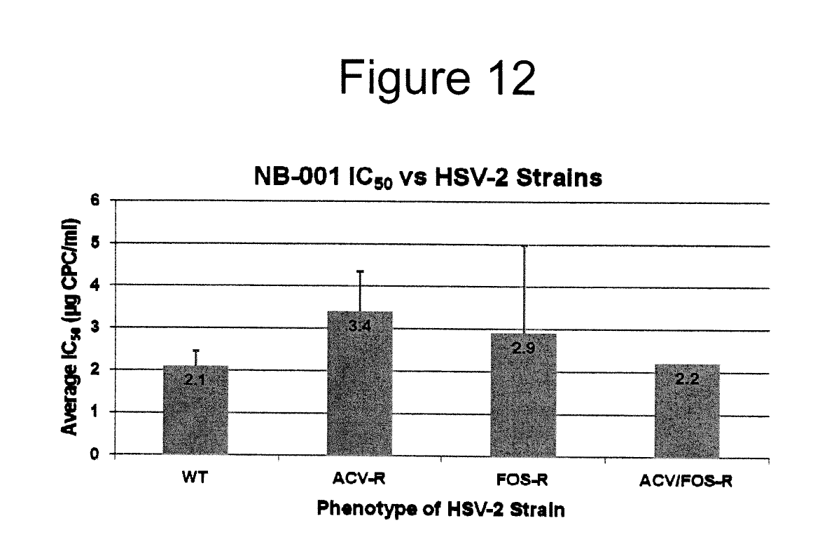

The nanoemulsions of the invention may be therapeutically effective against the herpes virus, viricidal against the herpes virus, viristatic against the herpes virus, or any combination thereof. See e.g., FIGS. 10, 11, and 12. FIG. 10 shows lysing of HSV-1 by a nanoemulsion according to the invention (NB-001; see Tables 5, 6, and 8 below for formulation details). FIG. 11 shows the inhibition of HSV-1 by a nanoemulsion according to the invention (NB-001). Finally, FIG. 12 shows the inhibition of HSV-2 by a nanoemulsion according to the invention (NB-001).

FIGS. 11 and 12 show NB-001 was equally virucidal against HSV-1 and HSV-2 strains, with a range of IC₅₀ values of 0.5-4.3 μg/mL. There was no cross-resistance to NB-001 when mutations conferring resistance to either the nucleoside analogue acyclovir (ACV) or the pyrophosphate analogue, foscarnet (FOS) were tested. Although HSV-2 strains are most commonly found in genital herpes, HSV-1 is the most common cause of newly diagnosed genital herpes in developed countries. HSV-2 is also know to cause herpes labialis.

The nanoemulsion droplets may traverse and/or diffuse through the epidermis, dermis, skin, skin pores, mucosa, cornea, compromised skin, nail, scalp, damaged skin, diseased skin, lateral or proximal folds, hyponichium, cornea or any combination thereof. Thus, the “topical” application can be to any superficial skin structure, eye, or any combination thereof.

The nanoemulsions comprise droplets having an average diameter of less than about 1000 nm, and the nanoemulsions comprise an aqueous phase, at least one oil, at least one surfactant or detergent, and at least one organic solvent. In one embodiment of the invention, the surfactant present in the nanoemulsion is a cationic surfactant. More than one surfactant or detergent can be present in the nanoemulsions of the invention. For example, the nanoemulsions can comprise a cationic surfactant in combination with a non-ionic surfactant. In another embodiment of the invention, the nanoemulsion further comprises a chelating agent. The “topical” application can be to any superficial skin structure, hair, hair shaft, hair follicle, eye, or any combination thereof. The organic solvent of the invention can be a non-phosphate based solvent.

In a further embodiment of the invention, a nanoemulsion additional comprises an active agent useful in treating, healing or palliating a herpes virus, such as an antiviral agent. Any suitable active agent, such as any antiviral agent suitable for treating a herpes infection, can be incorporated into the topical nanoemulsions of the invention. The nanoemulsion in and of itself has anti-viral activity and does not need to be combined with another active agent, such as a small molecule antiviral agent, to obtain therapeutic effectiveness. However, addition of another agent may enhance the therapeutic effectiveness of the nanoemulsion.

In one embodiment of the invention, the nanoemulsion comprises: (a) an aqueous phase; (b) about 1% oil to about 80% oil; (c) about 0.1% organic solvent to about 50% organic solvent; (d) about 0.001% surfactant or detergent to about 10% surfactant or detergent; (e) about 0.0005% to about 0.72% of a chelating agent; or (e) any combination thereof. In another embodiment of the invention, the nanoemulsion comprises: (a) about 10% oil to about 80% oil; (b) about 1% organic solvent to about 50% organic solvent; (c) at least one non-ionic surfactant present in an amount of about 0.1% to about 10%; (d) at least one cationic agent present in an amount of about 0.01% to about 2%; (e) about 0.0005% to about 1% of a chelating agent; or (f) any combination thereof.

These quantities of each component present in the nanoemulsion refer to a therapeutic nanoemulsion, and not to a nanoemulsion to be tested in vitro. This is significant, as nanoemulsions tested in vitro generally have lower concentrations of oil, organic solvent, surfactant or detergent, and (if present) chelating agent than that present in a nanoemulsion intended for therapeutic use, e.g., topical use. This is because in vitro studies do not require the nanoemulsion droplets to traverse the skin. For topical (or intradermal) use, the concentrations of the components must be higher to result in a therapeutic nanoemulsion. However, the relative quantities of each component used in a nanoemulsion tested in vitro are applicable to a nanoemulsion to be used therapeutically and, therefore, in vitro quantities can be scaled up to prepare a therapeutic composition, and in vitro data is predictive of topical application success.

### Preferred Concentration of Nanoemulsion

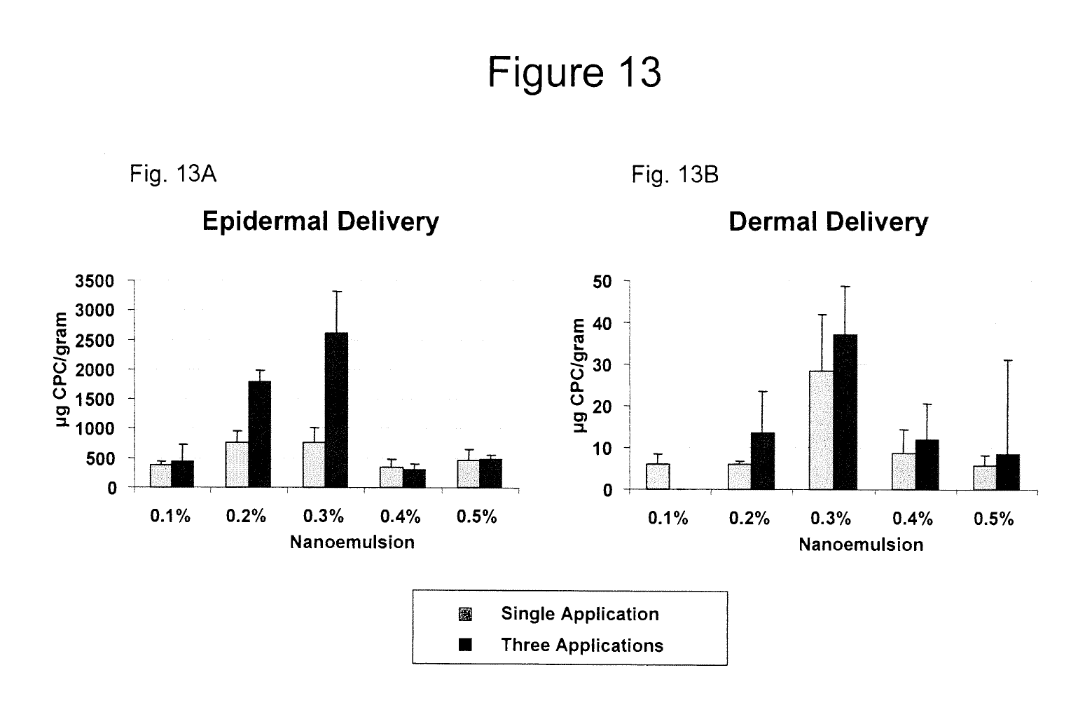

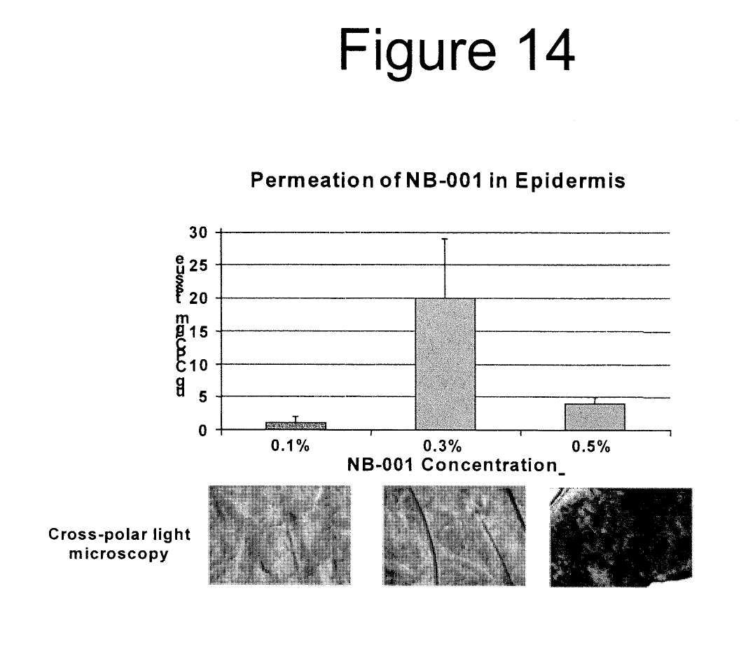

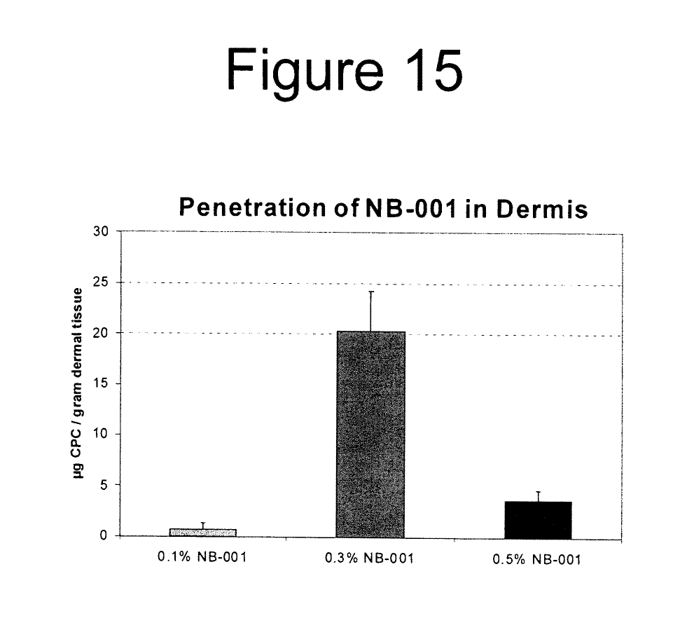

As shown in FIGS. 13-15, the concentration of nanoemulsion can vary. Interestingly, higher concentrations of nanoemulsion do not necessarily correspond to an increased effectiveness in the nanoemulsion of the invention. Measurement of a surfactant is used as marker of delivery in the examples of the invention, as the nanoemulsions do not contain a traditional “active agent” (although in another embodiment of the invention, an active can additionally be added to a nanoemulsion according to the invention). FIG. 13 shows the effect of higher nanoemulsion concentrations on delivery into pig skin. The results in FIGS. 14 and 15 show that optimal delivery is obtained utilizing a concentration of about 0.15 to about 0.35%, with a preferred concentration of about 0.2-0.3%, and most preferred of about 0.3% nanoemulsion.

### Crystallization as a Method of Limiting Absorption

At higher concentrations of nanoemulsion, i.e., greater than about 0.5%, the nanoemulsion tends to crystallize upon application to a surface, particularly after multiple applications of the nanoemulsion. This crystallization on the surface of the skin acts as a barrier to limit absorption of additionally applied nanoemulsion. See e.g., FIGS. 13-15. FIG. 13 shows that single and multiple applications of a nanoemulsion having a concentration of greater than 0.1% and less than 0.4% have optimal absorption. Surprisingly, increasing the concentration of the nanoemulsion does not increase absorption into the skin of the nanoemulsion. This effect becomes more pronounced with repeated applications of the nanoemulsion. As shown in FIGS. 14 and 15, multiple (5) applications of a nanoemulsion having a 0.3% concentration have a markedly greater absorption than multiple (5) applications of a nanoemulsion having a 0.5% concentration, for both epidermal (FIG. 14) and dermal (FIG. 15) absorption. This is because, as noted above, higher concentrations of nanoemulsion produce crystallization upon application, which produces a barrier on the skin. This barrier functions to limit absorption of additionally applied nanoemulsion. Such a barrier can be desirable as it can prevent excessive absorption of nanoemulsion. Higher concentrations of nanoemulsion can be desirable, depending upon the desired treatment and dose to be absorbed.

### Lateral Diffusion

As demonstrated in Examples 7 and 9 below, the nanoemulsions, as well as active agents incorporated into the nanoemulsions, diffuse translaterally within tissue planes to the site of infection without skin damage. Specifically, the examples below describe lateral diffusion of a nanoemulsion according to the invention along tissue planes to reach sites of infection up to 2 cm away from the site of skin application. This enables the treatment of infections present under barriers. Thus, a nanoemulsion according to the invention can be applied to a barrier covering an infection, and following application the nanoemulsion then migrates under (or laterally diffuses under) the barrier to effectively reach and eradicate the infection. This result is obtained without systemic absorption, as a measurable quantity of the nanoemulsion is not found within the plasma of a treated subject (determined by measuring if any surfactant or detergent, such as a cationic surfactant present in the nanoemulsion, is absorbed into the bloodstream).

Moreover, the examples show that an active agent incorporated within a nanoemulsion according to the invention diffuses laterally to areas not directly underlying the site of application. The suitable active agent includes, but not limited to, any antiviral agent or palliative agent, examples of which are described in Section D.6 below.

In addition, the data presented in the examples demonstrates that incorporating an active agent into a nanoemulsion results in unexpectedly superior delivery of the active agent, as compared to application of the active agent alone to the skin. Thus, an active agent incorporated into a nanoemulsion according to the invention appear to have synergistic activities, with the combination potentially producing significantly superior results as compared to each of the active agent and nanoemulsion applied separately. It is noted, however, that an active agent is not required to be incorporated into a nanoemulsion, as the nanoemulsion in and of itself has antiviral, viricidal, and other beneficial properties.

### A. Definitions

The present invention is described herein using several definitions, as set forth below and throughout the application.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The term “nanoemulsion,” as used herein, includes dispersions or droplets, as well as other lipid structures that can form as a result of hydrophobic forces that drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases.

The term “subject” as used herein refers to organisms to be treated by the compositions of the present invention. Such organisms include animals (domesticated animal species, wild animals), and humans.

The term “surfactant” refers to any molecule having both a polar head group, which energetically prefers solvation by water, and a hydrophobic tail which is not well solvated by water. The term “cationic surfactant” refers to a surfactant with a cationic head group. The term “anionic surfactant” refers to a surfactant with an anionic head group.

The terms “Hydrophile-Lipophile Balance Index Number” and “HLB Index Number” refer to an index for correlating the chemical structure of surfactant molecules with their surface activity. The HLB Index Number may be calculated by a variety of empirical formulas as described by Meyers, (Meyers, Surfactant Science and Technology, VCH Publishers Inc., New York, pp. 231-245 [1992]), incorporated herein by reference. As used herein, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 70 or more for commercial surfactants. Hydrophilic surfactants with high solubility in water and solubilizing properties are at the high end of the scale, while surfactants with low solubility in water which are good solubilizers of water in oils are at the low end of the scale.

The terms “buffer” or “buffering agents” refer to materials which when added to a solution, cause the solution to resist changes in pH.

The terms “chelator” or “chelating agent” refer to any materials having more than one atom with a lone pair of electrons that are available to bond to a metal ion.

The terms “pharmaceutically acceptable” or “pharmacologically acceptable,” as used herein, refer to compositions that do not substantially produce adverse allergic or immunological reactions when administered to a host (e.g., an animal or a human). Such formulations include dips, sprays, seed dressings, stem injections, sprays, and mists. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

As used herein, the term “topically” refers to application of the compositions of the present invention to the surface of the skin and mucosal cells and tissues (e.g., alveolar, buccal, lingual, sublingual, masticatory, or nasal mucosa, and other tissues and cells which line hollow organs or body cavities).

As used herein, the term “topically active agents” refers to compositions of the present invention that are applied to skin or mucosal surfaces. Desired pharmacological results are intended at or near the site of application (contact) to a subject

As used herein, the term “systemically active drugs” is used broadly to indicate a substance or composition whose administration is not necessarily near the infection source and whose levels can be measured at sites quite distant from the site of administration (e.g., oral drug administration where levels of the drug are found in the bloodstream or in tissues or organs).

### B. Properties of the Nanoemulsions of the Invention

The nanoemulsion of the invention effectively treats and/or controls a Herpes virus infection without being systemically absorbed and/or without irritating the epithelium. The nanoemulsion droplets can traverse the skin pores and hair follicles. The nanoemulsion effectively treats the Herpes virus infection by killing or inhibiting the growth of the virus, causing the Herpes virus to lyse, die, lose pathogenicity, or any combination thereof.

The nanoemulsion may be viricidal against the Herpes virus, viristatic against the Herpes virus, or a combination thereof. A method for determining the minimum virucidal concentration (MVC) of a nanoemulsion according to the invention can be modeled from an international standard designated as E1052-96 (Standard Test Method for Efficacy of Antimicrobial Agents Against Viruses in Suspension) and published by the American Society for Testing and Materials International, 100 Barr Harbor Drive, PO Box C700, West Conshohocken, Pa. 19428-2959, United States. The minimum virucidal concentration (MVC) is determined using a range of nanoemulsion concentrations that are mixed with 1×10⁵ to 3×10⁷ plaque-forming units of herpes virus per milliliter for 15 minutes at room temperature. The MVC is defined as the lowest concentration of nanoemulsion that kills 99.9% of the virus. Controls to monitor cell cytotoxicity of the viral host cells (Vero cells) and neutralization of the nanoemulsion are included. Only conditions that are not cytotoxic to Vero cells and under which the nanoemulsion is neutralized are valid.

Further, the nanoemulsions of the invention can limit the potential for lesion outbreak and recurrence.

### C. Stability on Storage and after Application of the Nanoemulsions of the Invention

### 1. Storage Stability

The nanoemulsions of the invention can be stable at about 40° C. and about 75% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years.

In another embodiment of the invention, the nanoemulsions of the invention can be stable at about 25° C. and about 60% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

Further, the nanoemulsions of the invention can be stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

### 2. Stability Upon Application

The nanoemulsions of the invention are stable upon application, as surprisingly the nanoemulsions do not lose their physical structure upon application. Microscopic examination of skin surface following application of a nanoemulsion according to the invention demonstrates the physical integrity of the nanoemulsions of the invention. This physical integrity may result in the desired absorption observed with the nanoemulsions of the invention.

### D. Nanoemulsions

The term “nanoemulsion”, as defined herein, refers to a dispersion or droplet or any other lipid structure. Typical lipid structures contemplated in the invention include, but are not limited to, unilamellar, paucilamellar and multilamellar lipid vesicles, micelles and lamellar phases.

The nanoemulsion of the present invention comprises droplets having an average diameter size of less than about 1,000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 300 nm. In a different embodiment, the droplets have an average diameter size greater than about 50 nm or greater than about 70 nm, and less than or equal to about 125 nm.

1. Aqueous Phase

The aqueous phase can comprise any type of aqueous phase including, but not limited to, water (e.g., H₂O, distilled water, tap water) and solutions (e.g., phosphate buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate buffered saline (PBS). The aqueous phase may further be sterile and pyrogen free.

2. Organic Solvents

Organic solvents in the nanoemulsions of the invention include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

3. Oil Phase

The oil in the nanoemulsion of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

4. Surfactants/Detergents

The surfactant or detergent in the nanoemulsion of the invention can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that consist of a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N—N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quarternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the present invention are not limited to formulation with a particular cationic containing compound.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the nanoemulsion comprises a cationic surfactant, which can be cetylpyridinium chloride. In other embodiments of the invention, the nanoemulsion comprises a cationic surfactant, and the concentration of the cationic surfactant is less than about 5.0% and greater than about 0.001%. In yet another embodiment of the invention, the nanoemulsion comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the nanoemulsion is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the nanoemulsion is less than about 5.0% and greater than about 0.001%.

In another embodiment of the invention, the nanoemulsion comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.05% to about 7.0%, or the non-ionic surfactant is present in a concentration of about 0.5% to about 4%. In yet another embodiment of the invention, the nanoemulsion comprises a cationic surfactant present in a concentration of about 0.01% to about 2%, in combination with a nonionic surfactant.

5. Additional Ingredients

Additional compounds suitable for use in the nanoemulsions of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion, or the additional compounds can be added to the original mixture to be emulsified. In certain of these embodiments, one or more additional compounds are admixed into an existing nanoemulsion composition immediately prior to its use.

Suitable preservatives in the nanoemulsions of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The nanoemulsion may further comprise at least one pH adjuster. Suitable pH adjusters in the nanoemulsion of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion can comprise a chelating agent. In one embodiment of the invention, the chelating agent is present in an amount of about 0.0005% to about 0.72%. Examples of chelating agents include, but are not limited to, phytic acid, polyphosphoric acid, citric acid, gluconic acid, acetic acid, lactic acid, ethylenediamine, ethylenediaminetetraacetic acid (EDTA), and dimercaprol, and a preferred chelating agent is ethylenediaminetetraacetic acid.

The nanoemulsion can comprise a buffering agent, such as a pharmaceutically acceptable buffering agent. Examples of buffering agents include, but are not limited to, 2-Amino-2-methyl-1,3-propanediol, ≧99.5% (NT), 2-Amino-2-methyl-1-propanol, ≧99.0% (GC), L-(+)-Tartaric acid, ≧99.5% (T), ACES, ≧99.5% (T), ADA, ≧99.0% (T), Acetic acid, ≧99.5% (GC/T), Acetic acid, for luminescence, ≧99.5% (GC/T), Ammonium acetate solution, for molecular biology, ˜5 M in H₂O, Ammonium acetate, for luminescence, ≧99.0% (calc. on dry substance, T), Ammonium bicarbonate, ≧99.5% (T), Ammonium citrate dibasic, ≧99.0% (T), Ammonium formate solution, 10 M in H₂O, Ammonium formate, ≧99.0% (calc. based on dry substance, NT), Ammonium oxalate monohydrate, ≧99.5% (RT), Ammonium phosphate dibasic solution, 2.5 M in H₂O, Ammonium phosphate dibasic, ≧99.0% (T), Ammonium phosphate monobasic solution, 2.5 M in H₂O, Ammonium phosphate monobasic, ≧99.5% (T), Ammonium sodium phosphate dibasic tetrahydrate, ≧99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≧99.5% (T), BES buffered saline, for molecular biology, 2× concentrate, BES, ≧99.5% (T), BES, for molecular biology, ≧99.5% (T), BICINE buffer Solution, for molecular biology, 1 M in H₂O, BICINE, ≧99.5% (T), BIS-TRIS, ≧99.0% (NT), Bicarbonate buffer solution, ≧0.1 M Na₂CO₃, ≧0.2 M NaHCO₃, Boric acid, ≧99.5% (T), Boric acid, for molecular biology, ≧99.5% (T), CAPS, ≧99.0% (TLC), CHES, ≧99.5% (T), Calcium acetate hydrate, ≧99.0% (calc. on dried material, KT), Calcium carbonate, precipitated, ≧99.0% (KT), Calcium citrate tribasic tetrahydrate, ≧98.0% (calc. on dry substance, KT), Citrate Concentrated Solution, for molecular biology, 1 M in H₂O, Citric acid, anhydrous, ≧99.5% (T), Citric acid, for luminescence, anhydrous, ≧99.5% (T), Diethanolamine, ≧99.5% (GC), EPPS, ≧99.0% (T), Ethylenediaminetetraacetic acid disodium salt dihydrate, for molecular biology, ≧99.0% (T), Formic acid solution, 1.0 M in H₂O, Gly-Gly-Gly, ≧99.0% (NT), Gly-Gly, ≧99.5% (NT), Glycine, ≧99.0% (NT), Glycine, for luminescence, ≧99.0% (NT), Glycine, for molecular biology, ≧99.0% (NT), HEPES buffered saline, for molecular biology, 2× concentrate, HEPES, ≧99.5% (T), HEPES, for molecular biology, ≧99.5% (T), Imidazole buffer Solution, 1 M in H₂O, Imidazole, ≧99.5% (GC), Imidazole, for luminescence, ≧99.5% (GC), Imidazole, for molecular biology, ≧99.5% (GC), Lipoprotein Refolding Buffer, Lithium acetate dihydrate, ≧99.0% (NT), Lithium citrate tribasic tetrahydrate, ≧99.5% (NT), MES hydrate, ≧99.5% (T), MES monohydrate, for luminescence, ≧99.5% (T), MES solution, for molecular biology, 0.5 M in H₂O, MOPS, ≧99.5% (T), MOPS, for luminescence, ≧99.5% (T), MOPS, for molecular biology, ≧99.5% (T), Magnesium acetate solution, for molecular biology, ˜1 M in H₂O, Magnesium acetate tetrahydrate, ≧99.0% (KT), Magnesium citrate tribasic nonahydrate, ≧98.0% (calc. based on dry substance, KT), Magnesium formate solution, 0.5 M in H₂O, Magnesium phosphate dibasic trihydrate, ≧98.0% (KT), Neutralization solution for the in-situ hybridization for in-situ hybridization, for molecular biology, Oxalic acid dihydrate, ≧99.5% (RT), PIPES, ≧99.5% (T), PIPES, for molecular biology, ≧99.5% (T), Phosphate buffered saline, solution (autoclaved), Phosphate buffered saline, washing buffer for peroxidase conjugates in Western Blotting, 10× concentrate, piperazine, anhydrous, ≧99.0% (T), Potassium D-tartrate monobasic, ≧99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≧99.0% (NT), Potassium acetate, for luminescence, ≧99.0% (NT), Potassium acetate, for molecular biology, ≧99.0% (NT), Potassium bicarbonate, ≧99.5% (T), Potassium carbonate, anhydrous, ≧99.0% (T), Potassium chloride, ≧99.5% (AT), Potassium citrate monobasic, ≧99.0% (dried material, NT), Potassium citrate tribasic solution, 1 M in H₂O, Potassium formate solution, 14 M in H₂O, Potassium formate, ≧99.5% (NT), Potassium oxalate monohydrate, ≧99.0% (RT), Potassium phosphate dibasic, anhydrous, ≧99.0% (T), Potassium phosphate dibasic, for luminescence, anhydrous, ≧99.0% (T), Potassium phosphate dibasic, for molecular biology, anhydrous, ≧99.0% (T), Potassium phosphate monobasic, anhydrous, ≧99.5% (T), Potassium phosphate monobasic, for molecular biology, anhydrous, ≧99.5% (T), Potassium phosphate tribasic monohydrate, ≧95% (T), Potassium phthalate monobasic, ≧99.5% (T), Potassium sodium tartrate solution, 1.5 M in H₂O, Potassium sodium tartrate tetrahydrate, ≧99.5% (NT), Potassium tetraborate tetrahydrate, ≧99.0% (T), Potassium tetraoxalate dihydrate, ≧99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≧99.5% (NT), Sodium acetate solution, for molecular biology, −3 M in H₂O, Sodium acetate trihydrate, ≧99.5% (NT), Sodium acetate, anhydrous, ≧99.0% (NT), Sodium acetate, for luminescence, anhydrous, ≧99.0% (NT), Sodium acetate, for molecular biology, anhydrous, ≧99.0% (NT), Sodium bicarbonate, ≧99.5% (T), Sodium bitartrate monohydrate, ≧99.0% (T), Sodium carbonate decahydrate, ≧99.5% (T), Sodium carbonate, anhydrous, ≧99.5% (calc. on dry substance, T), Sodium citrate monobasic, anhydrous, ≧99.5% (T), Sodium citrate tribasic dihydrate, ≧99.0% (NT), Sodium citrate tribasic dihydrate, for luminescence, ≧99.0% (NT), Sodium citrate tribasic dihydrate, for molecular biology, ≧99.5% (NT), Sodium formate solution, 8 M in H₂O, Sodium oxalate, ≧99.5% (RT), Sodium phosphate dibasic dihydrate, ≧99.0% (T), Sodium phosphate dibasic dihydrate, for luminescence, ≧99.0% (T), Sodium phosphate dibasic dihydrate, for molecular biology, ≧99.0% (T), Sodium phosphate dibasic dodecahydrate, ≧99.0% (T), Sodium phosphate dibasic solution, 0.5 M in H₂O, Sodium phosphate dibasic, anhydrous, ≧99.5% (T), Sodium phosphate dibasic, for molecular biology, ≧99.5% (T), Sodium phosphate monobasic dihydrate, ≧99.0% (T), Sodium phosphate monobasic dihydrate, for molecular biology, ≧99.0% (T), Sodium phosphate monobasic monohydrate, for molecular biology, ≧99.5% (T), Sodium phosphate monobasic solution, 5 M in H₂O, Sodium pyrophosphate dibasic, ≧99.0% (T), Sodium pyrophosphate tetrabasic decahydrate, ≧99.5% (T), Sodium tartrate dibasic dihydrate, ≧99.0% (NT), Sodium tartrate dibasic solution, 1.5 M in H₂O (colorless solution at 20° C.), Sodium tetraborate decahydrate, ≧99.5% (T), TAPS, ≧99.5% (T), TES, ≧99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate—EDTA buffer solution, for molecular biology, TRIS buffered saline, 10× concentrate, TRIS glycine SDS buffer solution, for electrophoresis, 10× concentrate, TRIS phosphate—EDTA buffer solution, for molecular biology, concentrate, 10× concentrate, Tricine, ≧99.5% (NT), Triethanolamine, ≧99.5% (GC), Triethylamine, ≧99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≧99.0% (NT), Trizma® base, ≧99.8% (T), Trizma® base, ≧99.8% (T), Trizma® base, for luminescence, ≧99.8% (T), Trizma® base, for molecular biology, ≧99.8% (T), Trizma® carbonate, ≧98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≧99.0% (AT), Trizma® hydrochloride, for luminescence, ≧99.0% (AT), Trizma® hydrochloride, for molecular biology, ≧99.0% (AT), and Trizma® maleate, ≧99.5% (NT).

The nanoemulsion can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsions that may readily be diluted with water to a desired concentration without impairing their antiviral properties.

6. Active Agents Incorporated into a Nanoemulsion of the Invention

In a further embodiment of the invention, a nanoemulsion additional comprises an active agent, such as an antiviral agent or a palliative agent. Addition of another agent may enhance the therapeutic effectiveness of the nanoemulsion. The nanoemulsion in and of itself has anti-viral activity and does not need to be combined with another active agent to obtain therapeutic effectiveness. Any antiviral agent suitable for treating a herpes infection can be incorporated into the topical nanoemulsions of the invention.

Examples of such antiviral agents include, but are not limited to, nucleoside analogs (e.g., acyclovir (Zovirax®), famciclovir (Famvir®), and valaciclovir (Valtrex®)), amantadine (Symmetrel®), oseltamivir (Tamiflu®), rimantidine (Flumadine®), and zanamivir (Relenza®), Cidofovir (Vistide®), foscarnet (Foscavir®), ganciclovir (Cytovene®), ribavirin (Virazole®), penciclovir (Denavir®), buciclovir, acyclic guanosine derivatives, (E)-5-(2-bromovinyl)-2′-deoxyuridine and structurally related analogues thereof [i.e., the cytosine derivative (E)-5-(2-bromovinyl)-2′-deoxycytidine and the 4′-thio derivative (E)-5-(2-bromovinyl)-2′-deoxy-4′-thiouridine], Nucleoside/Nucleotide Analogues (e.g., Abacavir (Ziagen, ABC), Didanosine (Videx, ddI), Emtricitabine (Emtriva, FTC), Lamivudine (Epivir, 3TC), Stavudine (Zerit, d4T), Tenofovir (Viread, TDF), Zalcitabine (Hivid, ddC), and Zidovudine (Retrovir, AZT, ZDV)); Nonnucleoside Reverse Transcriptase Inhibitors (e.g., Delavirdine (Rescriptor, DLV), Efavirenz (Sustiva, Stocrin, EFV), Etravirine (Intelence, TMC 125), Nevirapine (Viramune, NVP)); Protease Inhibitors (Amprenavir (Agenerase, APV), Atazanavir (Reyataz, ATV), Darunavir (Prezista, DRV, TMC 114), Fosamprenavir (Lexiva, Telzir, FPV), Indinavir (Crixivan, IDV), Lopinavir/Ritonavir (Kaletra), Nelfinavir (Viracept, NFV), Ritonavir (Norvir, RTV), Saquinavir (Invirase, SQV), and Tipranavir (Aptivus, TPV)); Fusion Inhibitors (e.g., Enfuvirtide (Fuzeon, ENF, T-20)); Chemokine Coreceptor Antagonists (e.g., Maraviroc (Selzentry, Celsentri, MVC)); and Integrase Inhibitors (e.g., Raltegravir (Isentress, RAL)). Preferred antiviral agents for incorporation into a nanoemulsion include, but are not limited to, acyclovir (Zovirax®), penciclovir (Denavir®), famciclovir (Famvir®), and valaciclovir (Valtrex®).

Examples of palliative agents which may be incorporated into the nanoemulsions of the invention include, but are not limited to, menthol, camphor, phenol, allantoin, benzocaine, corticosteroids, phenol, zinc oxide, camphor, pramoxine, dimethicone, meradimate, octinoxate, octisalate, oxybenzone, dyclonine, alcohols (e.g., benzyl alcohol), mineral oil, propylene glycol, titanium dioxide, and magnesium stearate.

Other examplary active agents which can be incorporated into a nanoemulsion for treating herpes include, but are not limited to, docosanol (Abreva®).

### E. Pharmaceutical Compositions

The nanoemulsions of the invention may be formulated into pharmaceutical compositions that comprise the nanoemulsion in a therapeutically effective amount and suitable, pharmaceutically-acceptable excipients for topical or intradermal administration to a human subject in need thereof. Such excipients are well known in the art.

By the phrase “therapeutically effective amount” it is meant any amount of the nanoemulsion that is effective in treating the Herpes virus infection by killing or inhibiting the growth of the Herpes virus, causing the Herpes virus to lose pathogenicity, or any combination thereof.

Dosage forms for topical or intradermal administration include, but are not limited to, patches, ointments, creams, emulsions, liquids, lotions, gels, bioadhesive gels, aerosols, shampoos, pastes, foams, sunscreens, capsules, microcapsules, or in the form of an article or carrier, such as a bandage, insert, syringe-like applicator, pessary, powder, talc or other solid, shampoo, cleanser (leave on and wash off product), and agents that favor penetration within the epidermis, the dermis and keratin layers.

Intradermal administration refers to injection of a nanoemulsion according to the invention between skin layers.

The pharmaceutical compositions may be formulated for immediate release, sustained release, controlled release, delayed release, or any combinations thereof, into the epidermis or dermis, with no systemic absorption. In some embodiments, the formulations may comprise a penetration-enhancing agent for enhancing penetration of the nanoemulsion through the stratum corneum and into the epidermis or dermis. Suitable penetration-enhancing agents include, but are not limited to, alcohols such as ethanol, triglycerides and aloe compositions. The amount of the penetration-enhancing agent may comprise from about 0.5% to about 40% by weight of the formulation.

The nanoemulsions of the invention can be applied and/or delivered utilizing electrophoretic delivery/electrophoresis. Such transdermal methods, which comprise applying an electrical current, are well known in the art.

Lack of systemic absorption may be monitored, for example, by measuring the amount of the surfactant, such as the cationic surfactant, in the plasma of the human subject undergoing treatment. Amounts of surfactant of equal to or less than about 10 ng/ml in the plasma confirms minimal systemic absorption. In another embodiment of the invention, minimal systemic absorption of the nanoemulsion occurs upon topical administration. Such minimal systemic exposure can be determined by the detection of less than 10 ng/mL, less than 8 ng/mL, less than 5 ng/mL, less than 4 ng/mL, less than 3 ng/mL, or less than 2 ng/mL of the one or more surfactants present in the nanoemulsion in the plasma of the subject.

The pharmaceutical compositions for topical or intradermal administration may be applied in a single administration or in multiple administrations. The pharmaceutical compositions are topically or intradermally applied for at least one day, at least two days at least three days at least four days at least 5 days, once a week, at least twice a week, at least once a day, at least twice a day, multiple times daily, multiple times weekly, biweekly, at least once a month, or any combination thereof. The pharmaceutical compositions are topically or intradermally applied for a period of time of about one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years. After application, the application area is washed to remove any residual nanoemulsion.

Following topical or intradermal administration, the nanoemulsion may be occluded or semi-occluded. Occlusion or semi-occlusion may be performed by overlaying a bandage, polyoleofin film, article of clothing, impermeabile barrier, or semi-impermeable barrier to the topical preparation.

### F. Exemplary Nanoemulsions

Several exemplary nanoemulsions are described below, although the methods of the invention are not limited to the use of such nanoemulsions. The components and quantity of each can be varied as described herein in the preparation of other nanoemulsions. (“CPC” refers to cetylpyridinium chloride, which is a cationic surfactant present in the nano emulsions.)

TABLE 4
Exemplary Therapeutically Effective Nanoemulsions

| | Soy- | Tween | | | | |
|---|---|---|---|---|---|---|
| Form. | bean | 20 | Ethanol | CPC % | EDTA | H₂O |
| (CPC %) | oil (%) | (%) | (%) | (mg/mL) | % (mM) | (%) |
| Formulation | 6.28 | 0.59 | 0.67 | 0.107 (1) | 0.0074 | 92.3 |
| #1; 0.1% | | | | | (0.2) | |
| Formulation | 18.84 | 1.78 | 2.02 | 0.320 (3) | 0.0222 | 77.02 |
| #2; 0.3% | | | | | (0.6) | |
| Formulation | 31.4 | 2.96 | 3.37 | 0.534 (5) | 0.037 | 61.71 |
| #3; 0.5% | | | | | (1.0) | |

### G. Methods of Manufacture

The nanoemulsions of the invention can be formed using classic emulsion forming techniques. See e.g., U.S. 2004/0043041. See also U.S. Pat. Nos. 6,015,832, 6,506,803, 6,559,189, 6,635,676, and US Patent Publication No. 20040043041, all of which are incorporated by reference. In addition, methods of making emulsions are described in U.S. Pat. Nos. 5,103,497 and 4,895,452 (herein incorporated by reference). In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In an exemplary embodiment, the nanoemulsions used in the methods of the invention comprise droplets of an oily discontinuous phase dispersed in an aqueous continuous phase, such as water. The nanoemulsions of the invention are stable, and do not decompose even after long storage periods. Certain nanoemulsions of the invention are non-toxic and safe when swallowed, inhaled, or contacted to the skin of a subject.

The compositions of the invention can be produced in large quantities and are stable for many months at a broad range of temperatures. The nanomulsion can have textures ranging from that of a semi-solid cream to that of a thin lotion, and can be applied topically by hand, and can be sprayed onto a surface.

As stated above, at least a portion of the emulsion may be in the form of lipid structures including, but not limited to, unilamellar, multilamellar, and paucliamellar lipid vesicles, micelles, and lamellar phases.

The present invention contemplates that many variations of the described nanoemulsions will be useful in the methods of the present invention. To determine if a candidate nanoemulsion is suitable for use with the present invention, three criteria are analyzed. Using the methods and standards described herein, candidate emulsions can be easily tested to determine if they are suitable. First, the desired ingredients are prepared using the methods described herein, to determine if a nanoemulsion can be formed. If a nanoemulsion cannot be formed, the candidate is rejected. Second, the candidate nanoemulsion should form a stable emulsion. A nanoemulsion is stable if it remains in emulsion form for a sufficient period to allow its intended use. For example, for nanoemulsions that are to be stored, shipped, etc., it may be desired that the nanoemulsion remain in emulsion form for months to years. Typical nanoemulsions that are relatively unstable, will lose their form within a day. Third, the candidate nanoemulsion should have efficacy for its intended use. For example, the emulsions of the invention should kill or disable Herpes virus in vitro. To determine the suitability of a particular candidate nanoemulsion against a desired Herpes virus, the nanoemulsion is exposed to the Herpes virus for one or more time periods in a side-by-side experiment with an appropriate control sample (e.g., a negative control such as water) and determining if, and to what degree, the nanoemulsion kills or disables the Herpes virus.

The nanoemulsion of the invention can be provided in many different types of containers and delivery systems. For example, in some embodiments of the invention, the nanoemulsions are provided in a cream or other solid or semi-solid form. The nanoemulsions of the invention may be incorporated into hydrogel formulations.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles (e.g., pressurized spray bottles).

### H. Examples

The invention is further described by reference to the following examples, which are provided for illustration only. The invention is not limited to the examples, but rather includes all variations that are evident from the teachings provided herein. All publicly available documents referenced herein, including but not limited to U.S. patents, are specifically incorporated by reference.

### EXAMPLES

### Example 1

### Phase 2A Study Regarding the Use of a Nanoemulsion to Treat Herpes Labialis

Three different nanoemulsions were prepared, all comprising soybean oil, Tween 20, ethanol, CPC, EDTA, and water. The formulations are summarized in Tables 5 and 6 below.

TABLE 5

| | | Tween | | | EDTA | |
|---|---|---|---|---|---|---|
| Form. | Soybean | 20 | Ethanol | CPC % | % | H₂O |
| (CPC %) | oil (%) | (%) | (%) | (mg/mL) | (mM) | (%) |
| Formulation | 1.57 | 0.15 | 0.17 | 0.027 | 0.0019 | 98.09 |
| #1; 0.025% | | | | (0.025) | (0.05) | |
| Formulation | 3.14 | 0.30 | 0.34 | 0.053 | 0.0037 | 96.17 |
| #2; 0.05% | | | | (0.5) | (0.1) | |
| Formulation | 6.28 | 0.59 | 0.67 | 0.107 | 0.0075 | 92.34 |
| #3; 0.1% | | | | (1) | (0.2) | |

TABLE 6
Nanoemulsion Formulations Used in Clinical Trials

| | | Emulsion | CPC |
|---|---|---|---|
| | Dilution | Concentration | Concentration |
| Murine Herpes Model | 1:50 | 2% | 0.02% |

Doses in Herpes Labialis Phase 2A

| | | | |
|---|---|---|---|
| 0.025% NB-001 | 1:40 | 2.5% | 0.025% |
| 0.05% NB-001 | 1:20 | 5% | 0.05% |
| 0.10% NB-001 | 1:10 | 10% | 0.10% |

Doses in Herpes Labialis Phase 2B

| | | | |
|---|---|---|---|
| 0.10% NB-001 | 1:10 | 10% | 0.10%* |
| 0.30 NB-001 | 1:3.3 | 30% | 0.30% |
| 0.50% NB-001 | 1:2 | 50% | 0.50%** |

*Maximum CPC concentration monographed for OTC use in humans.
**Maximum CPC concentration tested in 9 month minipig study.

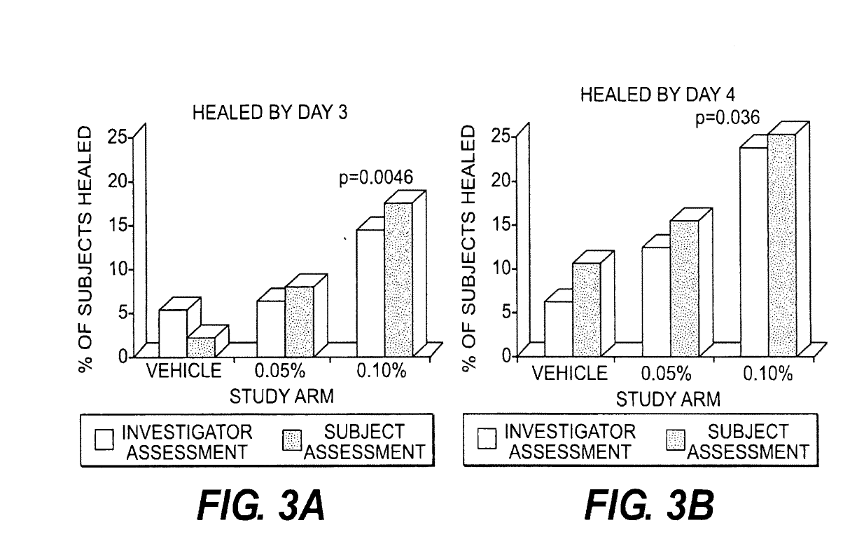

The study design was a randomized, controlled trial of the three different nanoemulsions as compared to a control (vehicle) in human subjects with recurrent Herpes Labialis. Locked kits with pre-randomized vials were given to 540 subjects. 332 subjects completed treatment. The results of the study are depicted in FIG. 3. Specifically, negligible blood absorption was detected in pharmacokinetic studies, and no skin irritation or drug-related adverse events were reported. Moreover, a 0.7 day improvement in mean time to healing as compared to the vehicle alone was observed.

Viral Swab Data

Site personnel obtained swabs of any lesion fluid present at each of the relevant office visits. A summary of viral swabs by day is presented in the Table below. Approximately half of the subjects in the trial had data that could be evaluated in this analysis due to difficulties with specimen collection, adequacy of specimens following shipment, and/or sensitivity of the PCR assay. The mean number of days with positive viral swabs was 0.5 in the NB-001 0.1% study group, 0.8 in the no treatment group, 0.8 in the NB-001 0.025% and 0.05% study groups, and 1.0 in the vehicle study group. In addition, the maximum number of days with virus reduced from 6 days to 4 days. Descriptively, subjects in the highest dose treatment arm became viral negative approximately one half day sooner than subjects in the vehicle control arm.

TABLE 7
Summary of Days with Positive Viral Swabs

| | No | | 0.025% | 0.05% | 0.10% | All |
|---|---|---|---|---|---|---|
| | Treatment | Vehicle | NB-001 | NB-001 | NB-001 | Subjects |
| | (N = 69) | (N = 59) | (N = 68) | (N = 66) | (N = 64) | (N = 326) |
| Number of | 36 | 29 | 38 | 30 | 34 | 167 |
| Subjects with | | | | | | |
| Evaluable | | | | | | |
| Viral swab | | | | | | |
| data | | | | | | |
| Days with | | | | | | |
| positive viral | | | | | | |
| swabs | | | | | | |
| mean | 0.8 | 1.0 | 0.8 | 0.8 | 0.5 | 0.8 |
| median | 0 | 0 | 0 | 0 | 0 | 0 |
| min | 0 | 0 | 0 | 0 | 0 | 0 |
| max | 6 | 4 | 5 | 5 | 4 | 6 |

### Example 2

### Efficacy in Humans

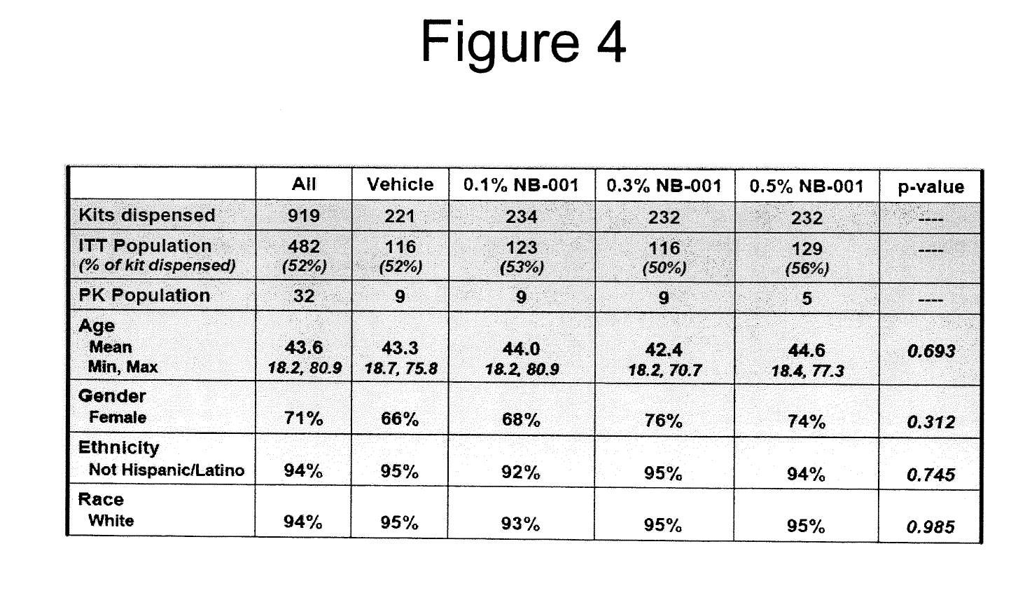

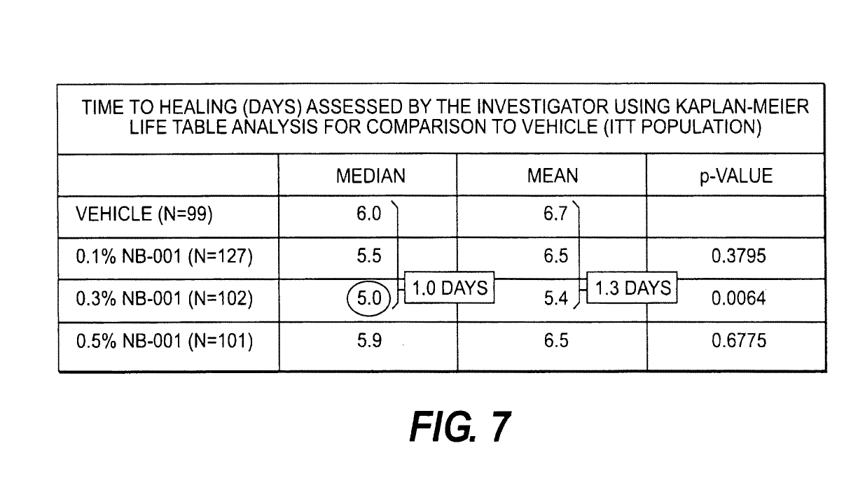

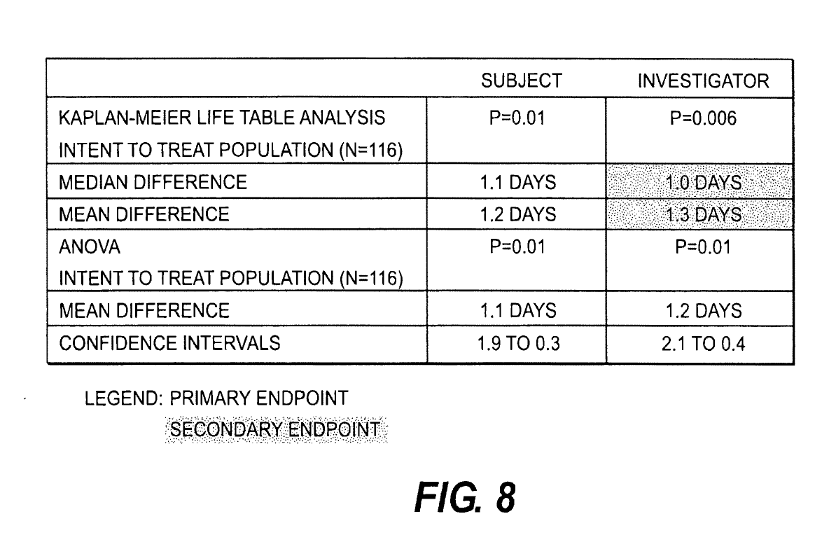

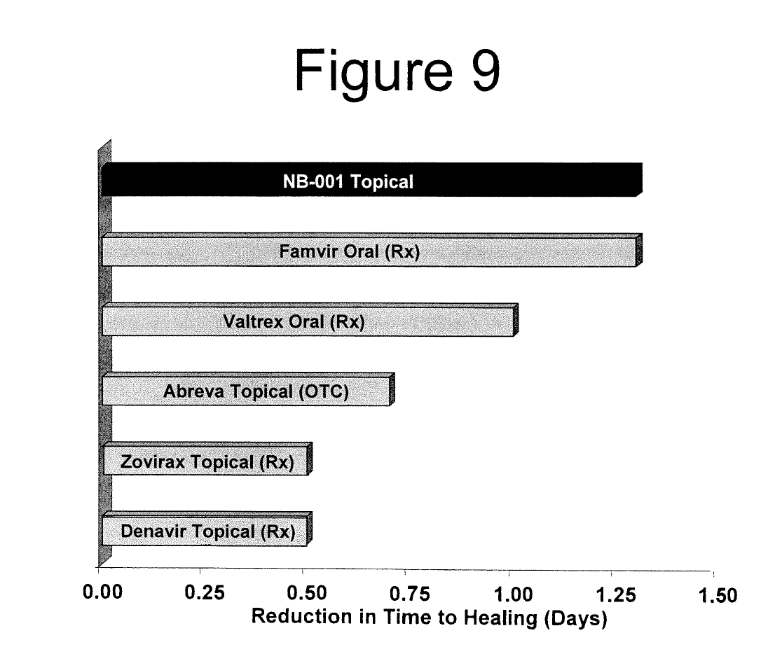

The purpose of this example was to determine the effectiveness of the nanoemulsions according to the invention in treating herpes labialis in humans. The results, shown in FIGS. 4-8, demonstrate efficacy and safety for the topical treatment of recurrent herpes labialis using the nanoemulsions. Moreover, the results also demonstrated efficacy equivalent to that of oral antiviral products. See FIG. 9. Most surprisingly, it was discovered that the 0.3% nanoemulsion improved time to healing by 1.3 days. No significant skin irritation, systemic absorption, or drug-related adverse events were recorded.

Three different nanoemulsions were prepared, all comprising soybean oil, Tween 20, ethanol, CPC, EDTA, and water. The formulations are summarized in Table 8 below. See also Tables 5 and 6.

TABLE 8

| | | Tween | | | EDTA | |
|---|---|---|---|---|---|---|
| Form. | Soybean | 20 | Ethanol | CPC % | % | H2O |
| (CPC %) | oil (%) | (%) | (%) | (mg/mL) | (mM) | (%) |
| Formulation | 6.28 | 0.59 | 0.67 | 0.107 (1) | 0.0074 | 92.3 |
| #1; 0.1% | | | | | (0.2) | |
| Formulation | 18.84 | 1.78 | 2.02 | 0.320 (3) | 0.0222 | 77.02 |
| #2; 0.3% | | | | | (0.6) | |
| Formulation | 31.4 | 2.96 | 3.37 | 0.534 (5) | 0.037 | 61.71 |
| #3; 0.5% | | | | | (1.0) | |

The nanoemulsions were utilized in a randomized, double-blind, placebo-controlled, dose-ranging trial of the three doses of the nanoemulsion compared with a control (vehicle). 28 U.S. sites distributed pre-randomized kits to 919 human subjects, ages 18 to 80, all with recurrent Herpes Labialis (FIG. 4). Each study participant had a history of at least three (3) cold sore outbreaks per year. 482 subjects had a cold sore attack, opened the kit, and started treatment. The nanoemulsion was applied 5× daily for four days, which is equivalent to 20 doses. The subjects were assessed twice daily by study investigators. The primary efficacy parameter was time to healing using a Kaplan-Meier analysis (ITT). Both a subject assessment and an investigator assessment were recorded.

### Introduction

A double-blind, vehicle controlled, dose-ranging Phase 2B study was performed in 482 subjects with recurrent labialis (cold sores). Subjects with a history of at least 3 cold sore outbreaks in the previous year received randomly assigned treatment kits containing either vehicle or NB-001 (0.1%, 0.3%, or 0.5%, corresponding to 0.1, 0.3, and 0.5% CPC). The nanoemulsions comprise Tween 20 as a surfactant, ethanol as an organic solvent, CPC as a cationic surfactant, soybean oil, DiH₂O, and EDTA. The exact amounts of each component are given in Table 8, above.

At the first onset of cold sore symptoms, subjects called an Interactive Voice Response System (IVRS) to receive a code to unlock their kit and started treatment 5 times a day for 4 days or until lesion healing. Subjects called the IVRS twice daily for lesion staging. Subjects returned to the clinic (Investigator assessment) within 12 hours of starting treatment and daily thereafter for lesion staging. Lesion stage was recorded as 0 (prodrome), 1 (erythema), blister (2), ulcer (3), scab (4), healed (5) or aborted (6). Subjects were allowed to entroll in the study regardless of baseline stage. Healing was defined as normal skin with no scab or crust and aborted was defined as prodrome or erythema without development of a lesion. Subjects called IVRS to record the date/time of healing or aborted lesion and returned to the clinic for confirmation of healing or aborted lesion.

Time to healing was determined from the date/time of treatment start to the date/time of lesion healing. The population enrolled in a cold sore study can significantly affect the endtime to healing.

### Results

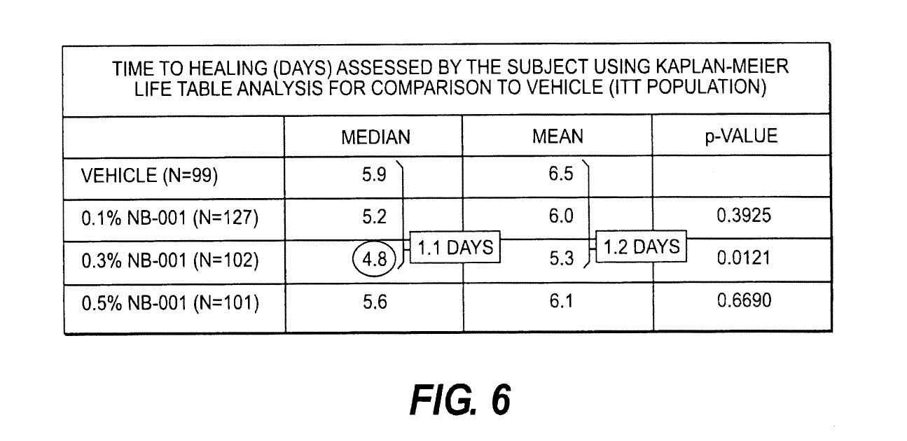

The results of this Phase 2 randomized, double-blind, vehicle-controlled, dose-response, study in 482 subjects demonstrate that 0.3% NB-001 administered as 0.2 mL 5 times daily for up to 4 days is effective in reducing the time to lesion healing in subjects with recurrent herpes labialis. Using life table methods, there were statistically significant reductions in time to healing for 0.3% NB-001 versus vehicle of 1.2 and 1.3 days based upon the subject (p=0.012) and the investigator (p=0.006) assessments, respectively (FIGS. 6-8). All results were statistically significant with a range of p-values from 0.0012-0.0486.

The treatment effect size of 0.3% NB-001 was numerically larger than the effect size seen with 0.1% NB-001 providing evidence of a dose response. The treatment effect size seen with 0.1% NB-001 in this study (0.5 days shortening of time to healing) was similar to that seen in a previous Phase 2 trial, but was not sufficient to achieve statistical significance with this sample size. Notably, there was no treatment effect 0.5% NB-001.

Previously reported studies on cold sore healing have used different entry criteria that can dramatically impact the time to healing. Of particular interest was a study of docosanol (Abreva®) published by Sacks that only allowed enrollment of subjects who did not have a blister at baseline. Sacks et al., “Clinical efficacy of topical docosanol 10% cream for herpes simplex labialis: A multicenter, randomized, placebo-controlled trial,” J. Am. Acad. Dermatol., 45:222-230 (2001). In the docosanol study, subjects who had the onset of cold sore symptoms (prodrome) were to report to the clinic and were only enrolled if they did not show evidence of a lesion at baseline. Subjects who do not have a lesion at baseline are thought to be more likely to have rapid lesion healing. Thus, studies who enroll only subjects without lesions at baseline tend to over estimate the treatment effect. In contrast, the NB-001-003 study allowed all subjects regardless of stage at baseline to begin treatment. In order to look at a population from the NB-001-003 study that was similar to the population in the docosanol study, we analyzed only the subset of NB-001-003 subjects who were assessed by the investigator as being at the prodrome or erythema stage at baseline.

Time to healing was determined from the date/time of a healed or aborted lesion minus the date/time of treatment start. In each of the four treatment groups (vehicle, 0.1%, 0.3%, 0.5%), the Kaplan-Meier method was used to summarize the distributions of time to healing. Subjects who did not have an investigator assessment at baseline were not included. Subjects who had a date of healing but no time recorded were assumed to have healed at 23 hours, 59 minutes on that date. Subjects who did not have a date/time of healing were considered as not healed at the last recorded date and time in the study.

A Cox proportional hazards regression model with time to healing as the dependent variable and treatment group as a factor was then fit. Pairwise comparisons between the vehicle group and each of the three active groups were then tested. The proportion of subjects with aborted lesions in each treatment group was calculated and the results compared descriptively. An aborted lesion was defined as a lesion that did not progress beyond the prodrome or erythema stage according to the investigator.

The prodrome and erythema subgroup consisted of 120 subjects, representing 24.8% of the total population enrolled in the study. The sample sizes in the vehicle, 0.1%, 0.3% and 0.5% groups were 27, 31, 34 and 28, respectively. Table 9 summarizes the distributions of time to healing in each of the four treatment groups.

TABLE 9
Kaplan-Meier Summary of Distributions of Time to Healing
(Days) in Subjects with Prodrome or Erythema at Baseline

| Treatment/Regimen | N | Median | p-value* |
|---|---|---|---|
| Vehicle | 27 | 5.3 | |
| 0.1% NB-001 5x/day | 31 | 3.9 | 0.376 |
| 0.3% NB-001 5x/day | 34 | 3.6 | 0.098 |
| 0.5% NB-001 5x/day | 28 | 4.5 | 0.385 |

*Comparison to vehicle

Although the sample size in each of the four treatment groups were small, the median times to healing demonstrate that healing tends to be faster in the 0.3% NB-001 group compared to vehicle (3.6 days vs. 5.3 days). Based on the Cox proportional hazards regression model, the two-sided p-values for the pairwise comparisons between the vehicle group and each of the active groups, indicate that, despite the small sample sizes, the difference between the 0.3% group and the vehicle group is nearly statistically significant.

The proportion of subjects with aborted lesions in each treatment group is shown in Table 10. Although no statistical comparisons were performed, the proportion of subjects with aborted lesions is numerically higher in the 0.3% NB-001 treatment arm.

TABLE 10
Proportion of Subjects with Aborted Lesions in the Subset
of Subjects with Prodrome or Erythema at Baseline

| | | Subjects with Aborted |
|---|---|---|
| Treatment/Regimen | N | Lesions (%) |
| Vehicle | 27 | 21.4 |
| 0.1% NB-001 5x/day | 31 | 19.4 |
| 0.3% NB-001 5x/day | 34 | 35.3 |
| 0.5% NB-001 5x/day | 28 | 17.9 |

An analysis of a subpopulation from the trial analogous to the subjects included in the published docosanol trial (Sacks et al.), i.e., subjects who did not have a lesion at baseline, indicated a median time to healing of 3.6 days for subjects in the 0.3% NB-001 treatment group, compared to 4.1 days reported in the docosanol study (Table 11).

TABLE 11
Comparison of docosanol (Abreva ®) and NB-001

| | |
|---|---|
| Docosanol (Abreva) | |
| Reported values | NB-001 |

| | Difference | | Difference |
|---|---|---|---|
| Time to | from Vehicle | Time to | from Vehicle |
| Heal (days) | (days) | Heal (d) | (days) |
| 4.1 | 0.73 | 3.6 | 1.7 |

Finally, Table 12 shows the Phase 2b results broken down with respect to quartiles.

TABLE 12
Quartile Results of the Phase 2B Study

| | 1^{st} | | 3^{rd} | |
|---|---|---|---|---|
| | Quartile | Median (CI) | Quartile | Mean (CI) |
| Vehicle | 3.8 | 5.9 (5.1, 6.5) | 8.3 | 6.5 (5.8, 7.1) |
| (N = 116) | | | | |
| 0.1% NB-001 | 3.7 | 5.2 (4.6, 6.1) | 7.8 | 6.0 (5.4, 6.5) |
| (N = 121) | | | | |
| 0.3% NB-001 | 3.1 | 4.8 (4.0, 5.3) | 7.0 | 5.3 (4.7, 5.8) |
| (N = 116) | | | | |
| 0.5% NB-001 | 3.9 | 5.6 (5.0, 6.1) | 8.0 | 6.1 (5.5, 6.6) |
| (N = 129) | | | | |

The results represent a 1.7 day improvement over vehicle for NB-001 treated subjects, as compared to less than a day improvement for subjects treated with docosanol. This suggests that starting treatment prior to the onset of a lesion, i.e., during the prodrome or erythema stage, could result in a greater treatment effect with a nanoemulsion according to the invention.

### Example 2

### The Nanoemulsions are Safe for Topical Application in Animals and Humans

In vivo safety studies were performed to confirm safety of the nanoemulsions for human use. The composition of the tested nanoemulsions is shown in Table 13.

TABLE 13

| Nano- | | | | | | |
|---|---|---|---|---|---|---|
| emulsion | | | | | | |
| (CPC | Soy- | | | | | |
| concen- | bean | Tween | Ethanol | CPC % | EDTA | % |
| tration) | oil % | 20 % | % | (mg/mL) | % (mM) | H₂O |
| 10 mg/mL | 62.79 | 5.92 | 6.73 | 1.068 | 0.0745 (2) | 23.42 |
| 5 mg/mL | 31.40 | 2.96 | 3.37 | 0.53 | 0.0373 (1) | 61.70 |
| 3 mg/mL | 18.84 | 1.78 | 2.02 | 0.320 | 0.0224 (0.6) | 77.03 |
| 1 mg/mL | 6.28 | 0.59 | 0.67 | 0.107 | 0.0075 (0.2) | 92.34 |
| 0 mg/mL | 12.56 | 1.18 | 1.35 | 0 | 0.0149 (0.4) | 84.90 |

10 female and 10 male guinea pigs were treated to determine if the nanoemulsions led to dermal—sensitization by administration of 10 mg/ml of the nanoemulsion three times weekly for three consecutive weeks, and then challenged for 6 hrs one week later. Dermal toxicity studies were also performed in groups of 4 female and 4 male minipigs that were subject to administration of 0.1-1 mg/cm² of the nanoemulsion daily for 9 months. Table 14 summarizes the results of the studies.

TABLE 14
Summary of Toxicity Studies

| | | | | Nano- | | | |
|---|---|---|---|---|---|---|---|
| | | | | emulsion | | Group | |
| Study | Species | Route | Dose | Conc. | Duration | Size | Findings |
| Dermal | Guinea | Topical | 0.3 ml/ | 10 mg/ml | Induction: 3 | 10/sex/ | No deaths |
| Sensitization | Pig | | chamber | | times weekly | group | occurred |
| | | | | | for 6 hours for | | No contact |
| | | | | | 3 consecutive | | sensitization |
| | | | | | weeks; | | occurred |
| | | | | | challenge for 6 | | |
| | | | | | hours | | |
| Chronic | Minipig | Topical | 0, 0.1, | 0, 1, 3, 5 mg/ml | 273-274 Days | 4/sex/ | No deaths |
| Dermal | | | 0.3, 05 mg/cm² | | | group | occurred |

Topical administration did not cause dermal sensitization in guinea pigs and showed no toxicity in a 9-month repeat dose dermal study in minipigs. These results clearly demonstrate that the nanoemulsions of the invention are safe for topical application.

### Human Safety

A double-blind, vehicle controlled, dose-ranging Phase 2B study was performed in 482 subjects with recurrent labialis (cold sores). Subjects with a history of at least 3 cold sore outbreaks in the previous year received randomly assigned treatment kits containing either vehicle or NB-001 (0.1%, 0.3%, or 0.5%, corresponding to 0.1, 0.3, and 0.5% CPC). Safety was also assessed in this trial. Throughout the studies, the skin around the lesion (i.e., skin not associated with the lesion) at the application site was assessed by the investigator (or trained study personnel) to determine the tolerability of the tissue to the study medication.

The nanoemulsions comprise Tween 20 as a surfactant, ethanol as an organic solvent, CPC as a cationic surfactant, soybean oil, DiH₂O, and EDTA. The exact amounts of each component are given in Table 8.

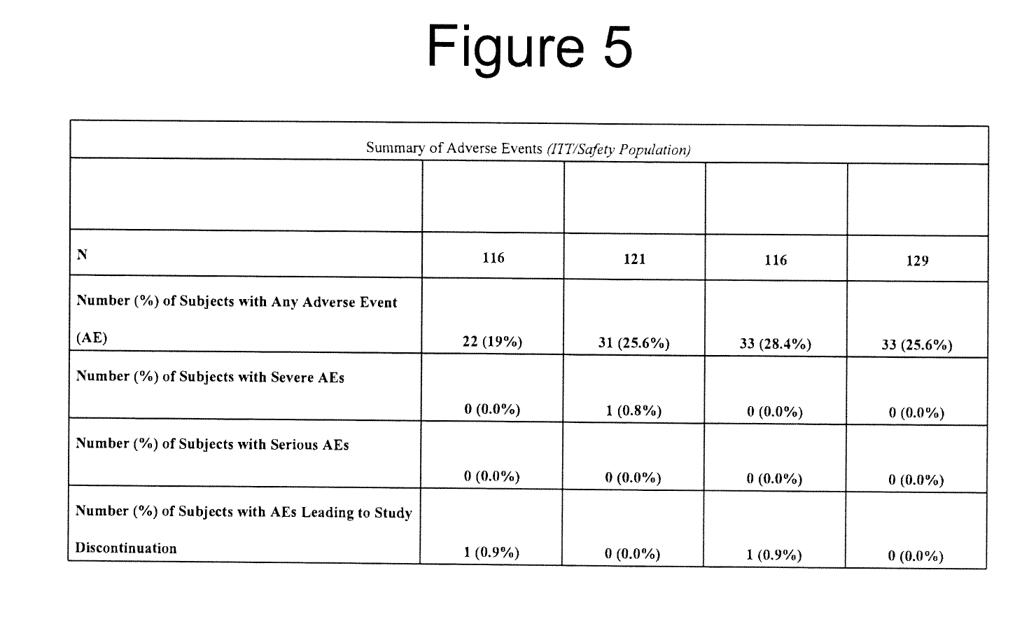

FIG. 5 shows a summary of adverse events reported in the trial. Table 15 shows the dermal adverse events reported for the trial. Overall, there were relatively few adverse events reported and these events were mild to moderate in severity and as expected for the study population. The overall incidence of administration site adverse events was low and there was no evidence of a dose response. All other adverse events occurred in less than 2% of subjects. There were no significant changes in hematology, serum chemistry or urinalysis parameters following treatment. Negligible levels of CPC (1.03-2.18 ng/mL) just over the limit of detection were found in isolated plasma samples from 4 subjects (2 in the vehicle arm, 1 in the 0.1% NB-001 arm and 1 in the 0.3% NB-001 arm), indicating no significant systemic absorption.

TABLE 15
NB-001: Dermal Adverse Events

| | Vehicle | 0.1% NB-001 | 0.3% NB-001 | 0.5% NB-001 |
|---|---|---|---|---|
| Treatment | (N = 116) | (N = 121) | (N = 116) | (N = 129) |
| Applica- | 0 (0.0%) | 1 (0.8%) | 1 (0.9%) | 0 (0.0%) |
| tion Site | | | | |
| Dryness | | | | |
| Applica- | 2 (2.7%) | 1 (0.8%) | 0 (0.0%) | 0 (0.0%) |
| tion Site | | | | |
| Irritation | | | | |
| Applica- | 4 (3.4%) | 2 (1.7%) | 1 (0.9%) | 0 (0.0%) |
| tion Site | | | | |
| Reaction | | | | |
| Edema | 0 (0.9%) | 1 (0.8%) | 0 (0.0%) | 1 (0.8%) |
| Peripheral | | | | |

These results clearly demonstrate that the nanoemulsions of the invention are safe for topical application.

### Example 3

### The Nanoemulsions Are Stable

The purpose of this example was to investigate the long term physiochemical stability of a nanoemulsion according to the invention.

Using validated analytical methods, three strengths (0.1% w/v, 0.25% w/v, and 0.5% w/v) of a nanoemulsion formulation (NB-001) was tested over a period of up to 36 months, at appropriate International Conference on Harmonization (ICH) storage conditions, to determine changes in potency, physical appearance, particle size distribution, and pH. Emulsion physical stability was assessed by monitoring changes in physical appearance (i.e., settling, creaming, color change, and phase separation). The nanoemulsions were assessed by general appearance (white homogenous liquid with no signs of separation), pH (4-6) by a pH meter, droplet size (<400 nm) by laser light diffraction light scattering using a Beckman Coulter N4 Particle Size Analyzer, and potency. The cationic surfactant present in the nanoemulsion, cetylpyridinium chloride, was used as the reporter of the potency of the nanoemulsion droplets and was quantitated by HPLC.

To assess long term stability, each strength was stored in glass vials at 25° C./60% RH and 5° C. for up to 36 months. Samples were analyzed at 0, 1, 3, 6, 9, 12, 18, 24, and 36 month intervals. Each strength was placed under stressed conditions (40° C./75% RH) for 6 months and analyzed at 1, 3, and 6 month time points. Given that the samples were stable at 40° C./75% RH, it was not necessary to test samples stored at the accelerated condition of 30° C./65% RH.

### Results

Physical and chemical stability was demonstrated for the three different strengths of nanoemulsion. No change was noted in the appearance of the nanoemulsions by visual inspection. In addition, there was no change in the mean particle size distribution or particle size. Particle size and particle size distribution met pre-set stability specifications, with a mean particle size of approximately 180 nm. There was no evidence of emulsion instability observed at any time point, including under stressed conditions. There was no change in the potency measurements or pH. Potency values showed little change from the 0.1% w/v and 0.5% w/v target initial values for the nanoemulsion.

### CONCLUSION

Stability data support a shelf life of up to three years for nanoemulsions according to the invention.

### Example 4

### The Nanoemulsions Diffuse Laterally to Sites of Infection

The purpose of this example was to test whether nanoemulsion droplets can diffuse laterally to areas in the skin not directly underlying the site of application.

In vitro studies were carried out using excised human cadaver skin in a modified Franz diffusion apparatus. The nanoemulsions used in this study were oil-in-water (o/w) emulsions with mean droplet diameters of ˜200 nm. The cetylpyridinium chloride (CPC), which is used as a marker for delivery, resides at the interface between the oil and water phases. Part of the surfactant is distributed in the oil core and part resides in the water phase.

The nanoemulsion test formulations comprised either 0.25% NB-002 or 0.5% NB-002 (“NB-002” comprises, in an aqueous medium, soybean oil, Tween 20® as a nonionic surfactant, ethanol, cetylpyridinium chloride (CPC) as a cationic surfactant, EDTA, and water). The emulsions were produced by mixing a water-immiscible oil phase with an aqueous phase followed by high energy emulsification to obtain the desired particle size of 200 nm. The aqueous CPC solution was prepared by simple weighing of the CPC and addition the water until the CPC was dissolved in the water phase. The composition of the nanoemulsions, expressed as w/w % unless otherwise noted, used in this study is given in Table 16 below.

TABLE 16
Compositions of the Nanoemulsions (NB-002) and the aqueous
CPC solution (AQ). The percentages are wt/wt, unless otherwise noted.

| | Soy- | | | | | |
|---|---|---|---|---|---|---|
| | bean | Tween | Ethanol | | EDTA | Water |
| Formulation | oil % | 20 % | % | CPC % | % (mM) | % |
| 0.50% | 31.4 | 2.96 | 3.37 | 0.53 | 0.037 (1) | 61.70 |
| NB002 | | | | | | |
| 0.25% | 15.7 | 1.48 | 1.68 | 0.27 | 0.0185 (0.5) | 80.85 |
| NB002 | | | | | | |
| 0.5% | 0 | 0 | 0 | 0.53 | 0 | 99.5 |
| w/vAQ | | | | | | |

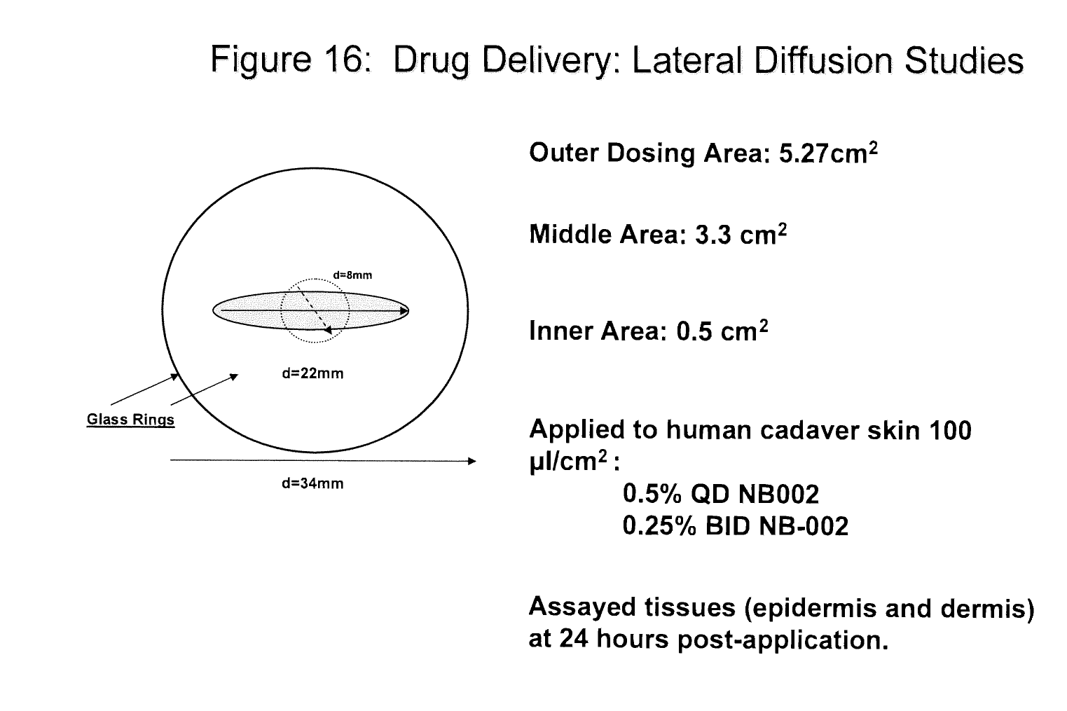

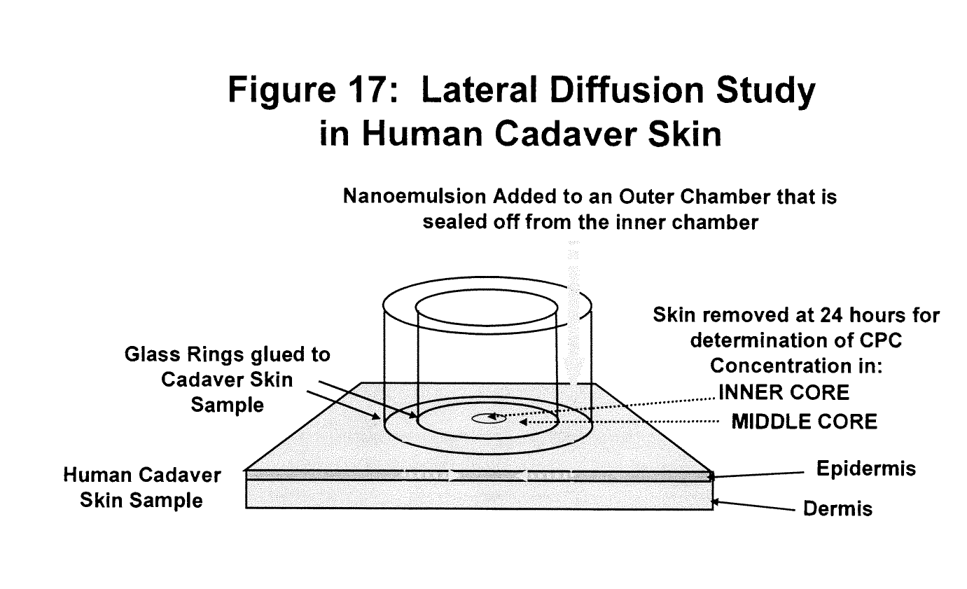

As described in more detail below, the NB-002 nanoemulsions at 100 μl/cm² were applied to a 5.27 cm² concentric surface area of skin enclosed by two concentric glass cylinders. See FIGS. 16 and 17. Twenty-four hours post application, residual nanoemulsion was removed by swabbing the dosing area. The epidermis and dermis of the dosing area was separated, weighed and assayed for CPC. An 8 mm (0.5 cm² surface area) punch biopsy of the inner non-dosing area (inner area) and middle non-dosing area (middle area) were processed in similar fashion. Quantification of CPC was performed by high pressure liquid chromatography (HPLC). Due to apparatus design, the only way CPC could be detected in the middle or inner tissues is through permeation of nanoemulsion into the skin underlying the dosing area traversing laterally into the non-dosing areas.

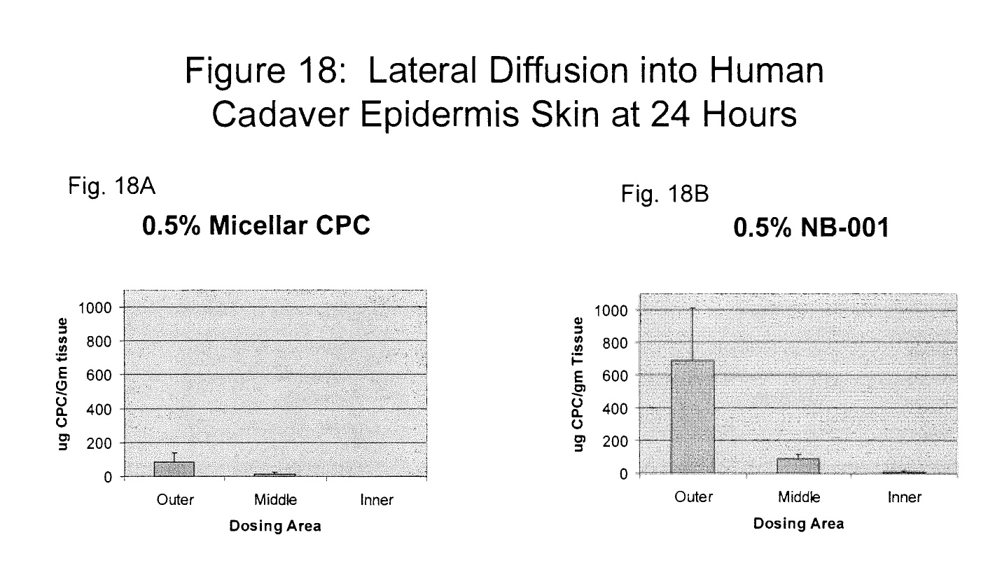

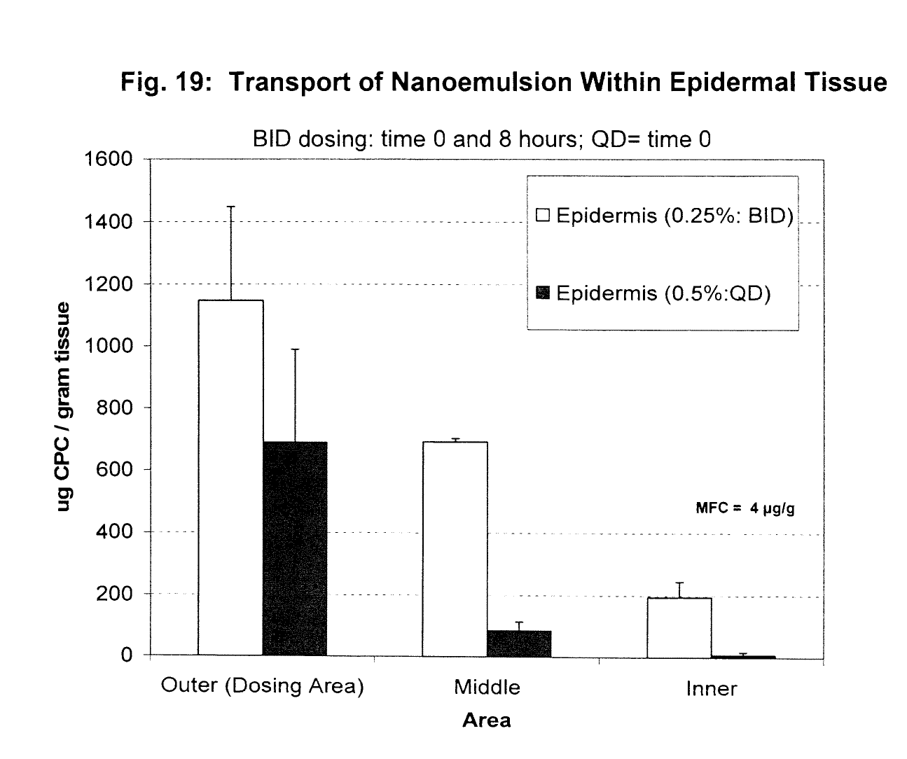

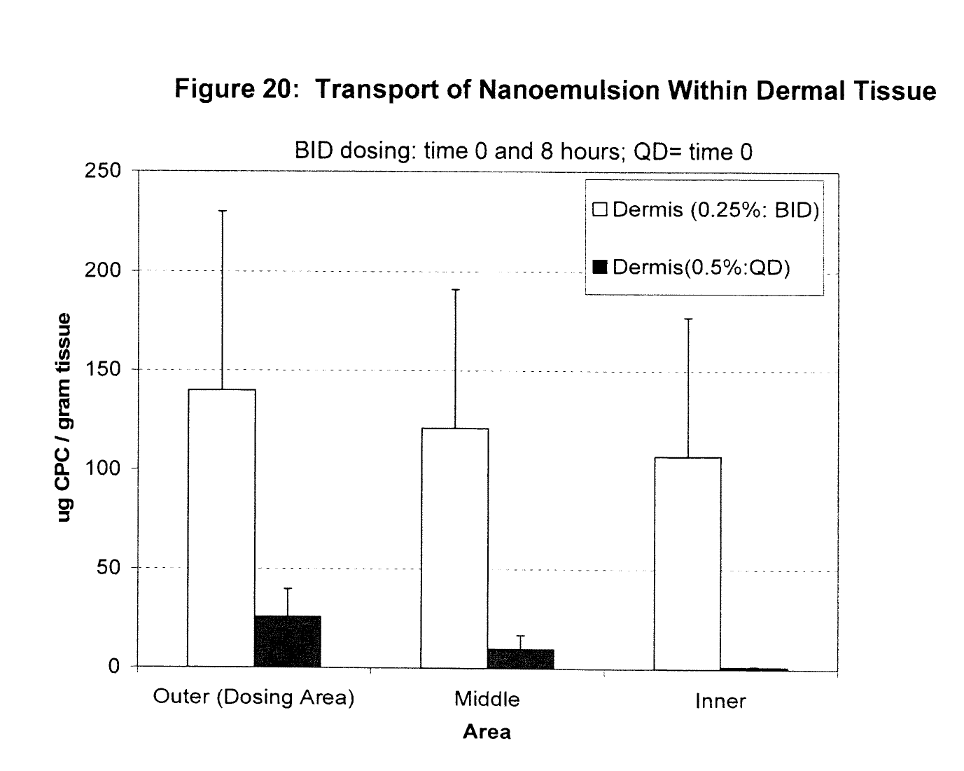

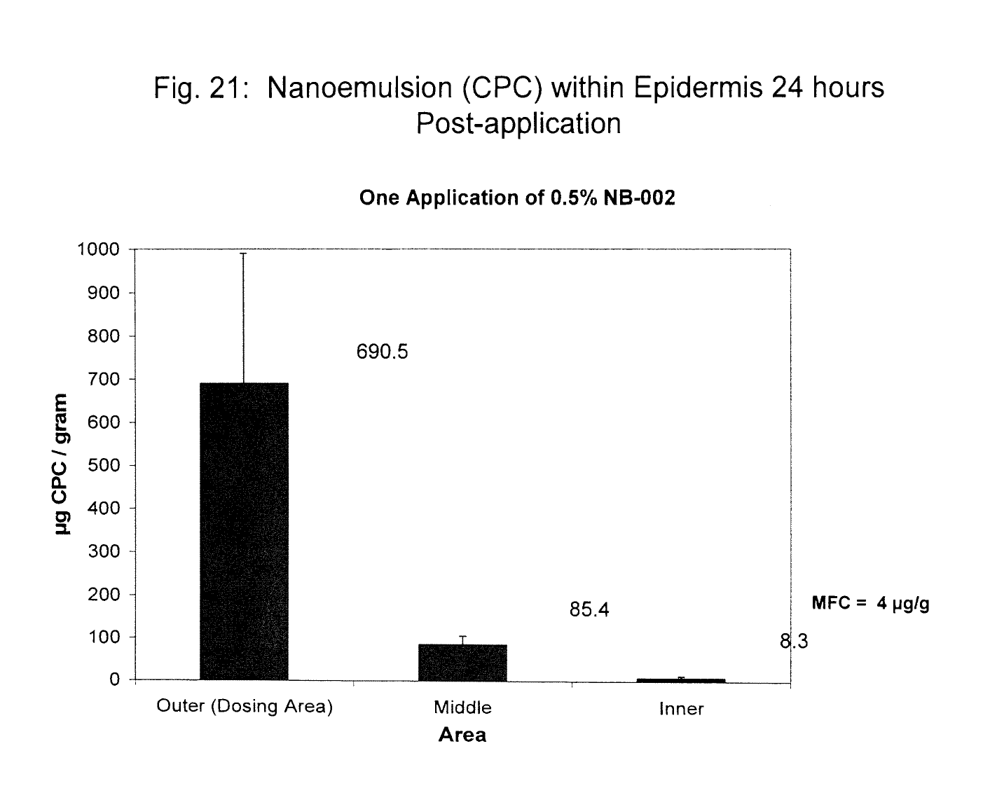

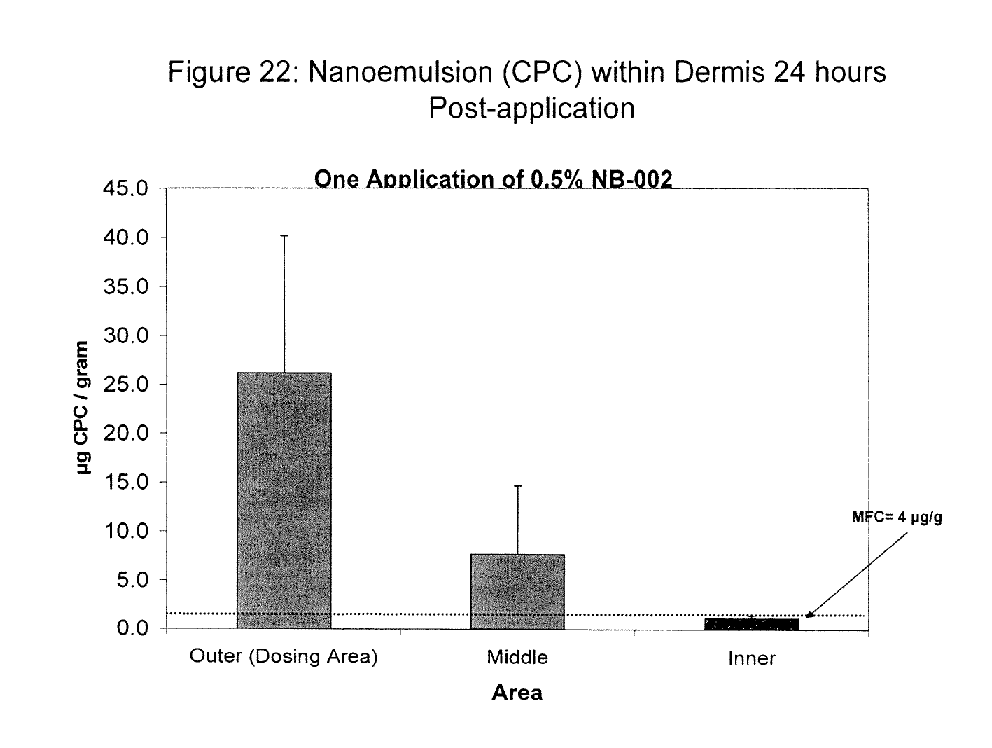

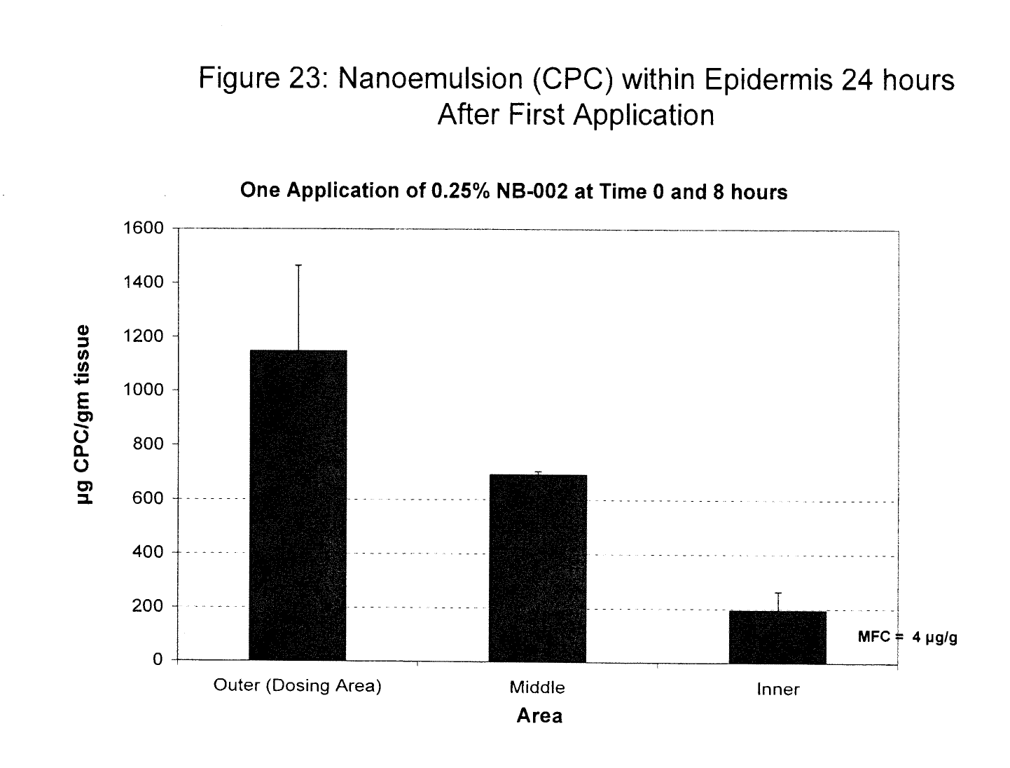

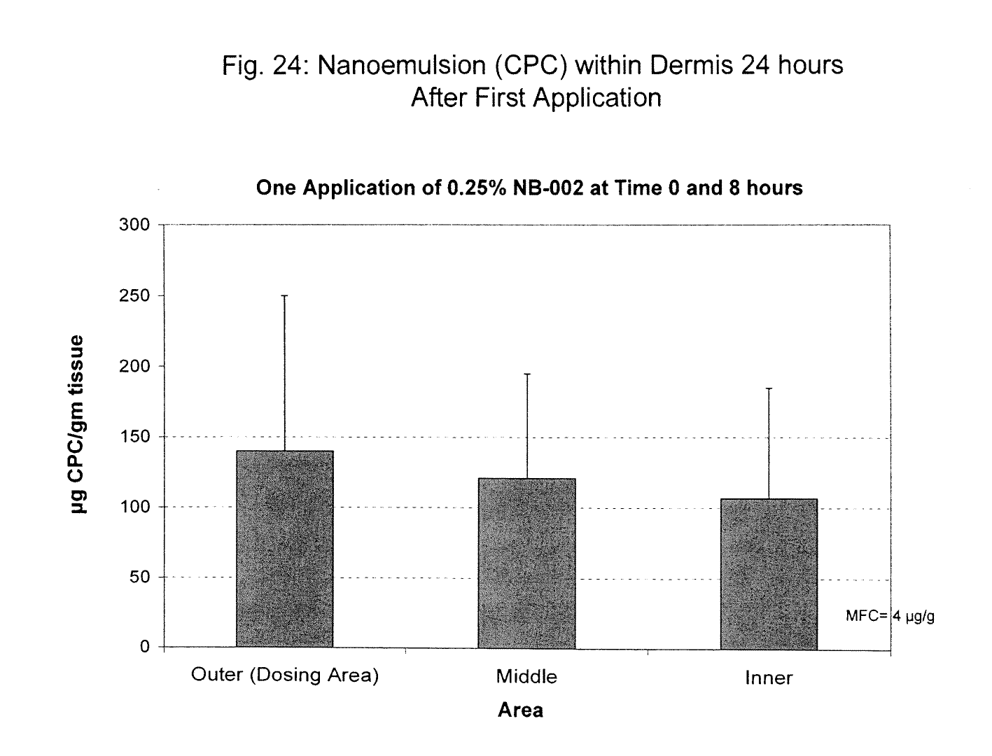

Epidermal and dermal concentrations of CPC in the non-dosing area were 700 and 150 μg/gram, respectively in the middle area and 200 and 100 μg/gram tissue, respectively, in the inner area. See FIGS. 18-24. These data indicate the nanoemulsion traversed laterally up to 11 mm from the dosing area. The levels of nanoemulsion in the middle and inner area tissues were substantially higher than the previously determined concentrations of nanoemulsion that kills fungi in vitro (4 μg/gram).

### EXPERIMENTAL

### Modified Diffusion Cell Methodology

Percutaneous absorption was measured using the in vitro cadaver skin finite dose technique. Cryopreserved, dermatomed (˜700 μm) human cadaver abdominal skin was used and stored in aluminum foil pouches at −70° C. until the time of use. At the time of use, the skin was thawed by placing the sealed pouch in 37° C. water for approximately five minutes. The skin was removed from the pouch and then cut into sections to fit on 38 mm permeation well cells. The receptor compartment was filled with distilled water, pH 7 and the donor compartment was left open to ambient laboratory conditions. All cells were mounted in a diffusion apparatus in which the receptor solution maintained at 37° C. by circulating water bath on the outside of the wells. The parameters for the diffusion study are listed in Table 17.

TABLE 17
Experimental Parameters

Parameters:

| | |
|---|---|
| Apparatus: | Permeation diffusion wells |
| Number of Cells: | 3-4 for 24 hours |
| Membrane: | Human Cadaver Abdominal Skin |
| Thickness: | ~700 μm |
| Overall Surface Diameter: | 38 mm |
| Duration: | 24 hours |
| Dosing Surface Area: | Outer dosing area, 5.27 cm² |
| Non-Dosing Area: | Inner non-dosing area, 0.5 cm² |
| | Middle non-dosing area, 3.3 cm² |
| Dose per surface area: | 100 μl/cm² |
| Concentration: | 0.5% w/v CPC in Aqueous solution |
| | 0.25% NB-002 |
| | 0.5% NB-002 |
| Cell Volume: | 50 ml |
| Receptor Solution: | Distilled water, pH 7.0 |
| Receptor Sampling: | 24 hours |
| Assay Method: | HPLC assay for CPC |
| Samples collected: | Surface swabs, Epidermis, Dermis, |
| | Receptor Samples |

Two circular glass chambers were glued using cyanoacrylate adhesive (e.g. super glue) was used to attach the chambers onto the skin surface as shown in FIG. 17. FIG. 16 illustrates the dimensions of the surface areas involved in the study. The test formulations were applied to the outer dosing area. The middle and inner areas did not receive a topical application of the test formulations.

The test formulations were applied to the epidermal surface of the donor chamber of the diffusion cells using a positive displacement pipette (μL). For single dosing 527 μl were applied (e.g. QD). For multiple dosing (e.g. BID), 527 μl was applied 8 hours after the initial dosing. The exposed dosing epidermal surface area was 5.27 cm².

At 24 hours after the first application, the outer dosing area was swabbed several times with 70% ethanol solution to remove all residual formulation from the skin surface. The surface area of the middle and inner areas were also swabbed. All the surface swabs were assayed for CPC content. The chambers were than removed and the outer dosing area was processed. Briefly, the epidermis was removed from the dermis in the outer dosing area via a scraping technique, placed in a tared vial and weighed. The dermis was than removed from the dosing area be using a scalpel and placed in a tared glass vial and weighed. All tissue weights were recorded and used in the calculations. The middle and inner areas were processed in the same fashion. The epidermal and dermal tissues from the outer, middle and inner areas were extracted with 70% ethanol solution, sonicated for 30 minutes, filtered through a 25 mm, 0.45 μm PTFE membrane syringe filter into HPLC vials and assayed using HPLC.

Receptor Medium

The receptor volume of each cell was 50 ml per apparatus. Distilled water, pH 7.0, was used as the receptor solution in the in vitro penetration studies. The receptor compartment spout was covered with parafilm to minimize evaporation of the receptor solution.

### RESULTS AND CONCLUSIONS

The results of permeation studies for NB-002 are shown in Tables 18 and 19. The levels of CPC found in the various compartments (epidermis, dermis and receptor) were significantly different for the aqueous CPC solution and the NB-002 formulations. The levels of CPC found in the epidermis and dermis after 24 hour duration were lower for the 0.5% w/v aqueous CPC solution as compared to the 0.25% and 0.5% NB-002. The amount of CPC found in the receptor compartment at 24 hours was below the level of detection (5 ng/ml) for all the formulations. More CPC was found in the epidermis and dermis from the 0.25% NB-002 formulation after twice daily application (applied t=0 and 8 hours later) as compared to the 0.5% NB-002 applied once.

TABLE 18
Epidermal Human cadaver skin summary (amount CPC (μg) per
weight tissue (g): mean of replicates ± SD). Percutaneous
absorption of CPC formulations through human cadaver skin
over 24 hours from a single or two dose topical applications.

| | 0.5% w/v | | |
|---|---|---|---|
| | Aqueous CPC, | 0.5% NB-001, | 0.25% NB-002, |
| Parameter | QD (μg/g) | QD (μg/g) | BID (μg/g) |
| Outer Dosing Area | 82.2 ± 58.6 | 690.5 ± 321.0 | 1148.0 ± 317 |
| Middle Area | 12.3 ± 10.6 | 85.4 ± 29.0 | 693 ± 11 |
| Inner Area | 0 | 8.32 ± 9.3 | 196 ± 68 |
| Receptor | 0 | 0 | 0 |
| Total Absorption | 94 | 784 | 2037 |
| (Epidermis, Dermis) | | | |

TABLE 19
Dermal Human cadaver skin summary (amount CPC (μg) per
weight tissue (g): mean of replicates ± SD). Percutaneous
absorption of CPC formulations through human cadaver skin
at 24 hours from a single topical or two topical applications.

| | 0.5% w/v | | |
|---|---|---|---|
| | Aqueous CPC, | 0.5% NB-001, | 0.25% NB-002, |
| Parameter | QD (μg/g) | QD (μg/g) | BID (μg/g) |
| Outer Dosing Area | 4.5 ± 1.1 | 26.1 ± 14 | 140 ± 110 |
| Middle Area | 1.7 ± 1.2 | 10 ± 7.4 | 121 ± 74 |
| Inner Area | 0 | 1.1 ± 0.3 | 107 ± 78 |
| Receptor Compartment | 0 | 0 | 0 |
| Total Absorption | 6.2 | 37 | 368 |
| (Epidermis, Dermis) | | | |

These results confirm that nanoemulsion diffuses laterally under the stratum corneum to tissues over a centimeter away from the site of application.

### Example 5

The purpose of this example was to evaluate the in vitro absorption into the epidermis and dermis of nanoemulsions according to the invention further comprising the active agent terbinafine hydrochloride (TB) as compared to that of the conventional TB formulation represented by Lamisil® cream. Pig skin was used as an animal model.

### 5.1: In Vitro Skin Model

The in vitro skin model has proven to be a valuable tool for the study of percutaneous absorption of topically applied compounds. The model uses excised skin mounted in specially designed diffusion chambers that allow the skin to be maintained at a temperature and humidity that match typical in vivo conditions. Franz, T J: Percutaneous absorption: on the relevance of in vitro data. J Invest Dermatol, 1975, 64:190-195. A finite dose of formulation is applied to the epidermis, outer surface of the skin and compound absorption is measured by monitoring its rate of appearance in the receptor solution bathing the dermal surface of the skin. Data defining total absorption, rate of absorption, as well as skin content can be accurately determined in this model. The method has historic precedent for accurately predicting in vivo percutaneous absorption kinetics. Franz T J: The finite dose technique as a valid in vitro model for the study of percutaneous absorption in man. In: Skin. Drug Application and Evaluation of Environmental Hazards, Current Problems in Dermatology, vol. 7, G. Simon, Z. Paster, M Klingberg, M. Kaye (Eds), Basel, Switzerland, S. Karger, 1978, pp 58-68.

### 5.2: Terbinafine Hydrochloride

Terbinafine hydrochloride is a white, fine crystalline, powder that is freely soluble in methanol and dichloromethane, soluble in ethanol, and slightly soluble in water. Terbinafine is mainly effective of the dermatophyte group of fungi. Oral tablets containing 250 mg TBHC are often prescribed for the treatment of onychomycosis of the toenail or fingernail due to the dermatophyte Tinea unguium. As a 1% cream or powder it is used for superficial skin infections such as jock itch (Tinea cruris), athlete's foot (Tinea pedis) and other types of ringworm (Tinea coporis). The chemical structure and physical chemical properties are given below.

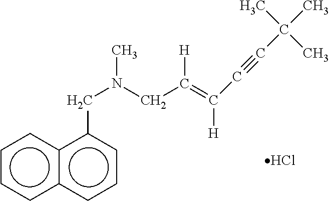

### 5.3 Nanoemulsions Used in the Study

Two different nanoemulsions were prepared. Nanoemulsion formulation #1 comprised 1% TB, 0.3% cetylpyridinium chloride (CPC), and 10% ethanol. Nanoemulsion formulation #2 comprised 1% TB, 0.3% cetylpyridinium chloride (CPC), and 20% ethanol. The Lamisil® cream comprises 1% TB. Nanoemulsions used in this study are oil-in-water (o/w) emulsions with mean droplet diameters of ˜180 nm. Cetylpyridinium chloride (CPC), a cationic surfactant in the nanoemulsion, was used as an additional marker agent of delivery. CPC resides at the interface between the oil and water phases. The hydrophobic tail of the surfactant distributes in the oil core and its polar head group resides in the water phase.

TABLE 20
Compositions of the Nanoemulsions
The percentages are wt/wt, unless otherwise noted.

| | Soybean | Tween | | CPC % | TBCH % | | |
|---|---|---|---|---|---|---|---|
| Formulation | oil % | 20 % | Ethanol % | (w/v) | (wt/v) | EDTA % | Water % |
| 1% TBHC/0.3% | 18.837 | 1.776 | 12.037 | 0.320 | 1.0 | 0.022 | 66.01 |
| nanoemulsion a | | | | | | | |
| 1% TBHC/0.3% | 18.837 | 1.776 | 22.037 | 0.320 | 1.0 | 0.022 | 56.01 |
| nanoemulsion b | | | | | | | |

### 5.4 Pig Skin

Full thickness, back skin (1000 μm thickness) from 2 month old male swine was used in permeation studies and obtained from Sinclair Research Center, Inc, Auxvasse, Mo. The subcutaneous fat was removed using a scalpel and the skin was stored in aluminum foil pouches at −70° C. until use. At time of use, the skin was thawed by placing the sealed pouch in 30° C. water for approximately five minutes. Thawed skin was removed from the pouch and cut into circular discs (30 mm diameter) to fit between the donor and receiver sides of the permeation chambers.

### 5.5 Franz Diffusion Cell Methodology: Conditions, Parameters, Procedure

Percutaneous absorption was measured using the in vitro cadaver skin finite dose technique². The receptor compartment was filled with distilled water, pH 7 and the donor compartment was left open to ambient laboratory conditions. The receptor volume of each cell was 7.7 ml per apparatus with a magnetic stirring bar. The receptor compartment spout was covered with a teflon screw cap to minimize evaporation of the receptor solution. Correctly-sized pig skin was placed onto the opening on the permeation cell. All cells were individually clamped with a clamp-support and placed in a heating bath which was maintained at 37° C. by a circulating water bath on the outside of the cells. The receptor compartment was maintained at 37° C. with the water bath and magnetic stirring. The surface temperature of the skin was appropriately 32° C. as determined by an IR surface temperature probe.

The skin was equilibrated for a period of 30 minutes before applying the 113 μL dose. The nanoemulsion formulations were applied onto the epidermal surface of the donor chamber of the diffusion cells using a positive displacement pipette. The exposed dosing epidermal surface area was 1.13 cm². A second dose of 113 μL was applied 8 hours later. The Lamisil^{AT} Cream was also applied using a positive displacement pipette and then rubbed into the skin for 10 seconds. The cream was also applied 8 hours later. Twenty four hours after application of the first dose, the surface of the skin was rinsed with 1 ml of 70% ethanol/water solution and then cleaned with a 70% ethanol soaked cotton swab, four times. Following alcohol swabbing, the donor cap was removed and the skin was removed from the apparatus. The epidermis was removed from the dermis via a scraping method and placed in a tarred scintillation vial. A punch biopsy was taken through the dermis and placed in a tarred scintillation vial. Weights of dermis and epidermis were recorded. The excess skin portion was placed in scintillation vial with the surface swabs.

### 5.6 Sampling (Receptor Sampling, Epidermis, Dermis, Surface Swabs/Extra Skin)

Twenty-four hours after application of the first dose, the surface of the dosing area was rinsed with 1 mL of 70% ethanol/water solution and swabbed independently several times with cotton swabs soaked 70% ethanol/water solution to remove all residual formulation from the skin surface. All the surface swabs were assayed for CPC content. Two mL of the receptor solution was also sampled at 24 hours from the receptor of each cell and filtered through a 0.45 μm PTFE (25 mm) membrane syringe filter into two HPLC snap cap vials and assayed independently for TBHC and CPC.

Skin samples were collected as described above; weights of the epidermal and dermal tissue were recorded. The epidermal and dermal tissues were extracted with 3 mL of 200 proof, absolute ethanol, sonicated for 30 minutes, filtered through a 25 mm, 0.45 μm PTFE membrane syringe filter into HPLC vials and assayed using HPLC. Samples were assayed for TBHC and CPC independently. Lamisil samples were also assayed for CPC, as a negative control.

### 5.7 Epidermal and Dermal Calculations

The amount of TBHC and CPC that permeated into the epidermis, dermis and the receptor compartment (at 24 hours after first dose) was determined by HPLC. A standard concentration of TBHC or CPC was generated and used to determine the concentration of TBHC or CPC in the dosing area. The levels of CPC or TBHC in each skin area are represented as: 1) amount per wet tissue weight (μg/grams)±the standard deviation; 2) amount per surface area (μg/cm²)±the standard deviation; 3) the % of the applied dose±the standard deviation. The number of replicas used in the calculation was 5 for each formulation.

In vitro skin permeation studies were performed using a Franz diffusion cell methodology. Twenty-four hours after two applications of three different test articles, the epidermis and dermis were separated, weighed and assayed for TBHC (e.g. Lamisil^{AT} Cream, 1% TBHC/0.3% nanoemulsion a, 1% TBHC/0.3% nanoemulsion b) by HPLC. The receptor samples were also assayed for TBHC. CPC was determined from the same samples from the NB-00X formulations as a marker of the nanoemulsion by HPLC.

### 5.8 CPC Levels Following Topical Administration of 1% TBHC/0.3% Nanoemulsion Formulations

The results of CPC permeation studies for 1% TBHC/0.3% nanoemulsion formulations are shown in Table 21.

TABLE 21
Percutaneous absorption of CPC formulations into pig skin over 24 hours from BID dosing.
Epidermal and dermal summary (amount CPC (μg) per surface area (cm²): mean of replicates ± SD;
amount CPC (μg) per weight tissue (g): mean of replicates ± SD); % of the total applied dose).

| | 1% TBHC/0.3% nanoemulsion a | | | 1% TBHC/0.3% nanoemulsion b | | |
|---|---|---|---|---|---|---|
| | | μg/gram | % applied | | | % applied |
| | μg/cm² | tissue | dose | μg/cm² | μg/g | dose |
| Epidermis | 48.8 ± 16.3 | 941.2 ± 437.3 | 8.14 ± 2.70 | 58.8 ± 12.9 | 1236.8 ± 242.7 | 9.80 ± 2.15 |
| Dermis | 9.1 ± 4.3 | 37.1 ± 17.1 | 1.52 ± 0.71 | 17.3 ± 5.7 | 70.6 ± 23.5 | 2.88 ± 0.95 |
| Receptor | 0 | 0 | 0 | 0 | 0 | 0 |

| | | |
|---|---|---|
| Mass Balance | 97.29 ± 2.22% | 98.86 ± 1.14% |

The delivery of the CPC marker into the epidermis with the 1% TBHC/0.3% nanoemulsion a and 0.3% nanoemulsion b were comparable. Ethanol concentration in the nanoemulsion formulation appears to enhance delivery of CPC into dermal tissues. 1% TBHC/0.3% nanoemulsion b formulation had 2 fold higher levels of CPC (37.1 μg/gram compared to 70.6 μg/gram) than the 1% TBHC/0.3% nanoemulsion a formulation. This finding is consistent with that seen with TBHC levels in the dermis.

The amount of CPC found in the receptor compartment at 24 hours was below the level of detection (5 ng/ml) for all the formulations.

### 5.9 TBCH Absorption Results

The results of TBHC permeation studies for Lamisil^{AT}, 1% TBHC/0.3% nanoemulsion a and 1% TBHC/0.3% nanoemulsion b are shown in Table 22.

TABLE 22
Percutaneous absorption of TBCH formulations into pig skin over 24 hours from BID dosing.
Epidermal and dermal pig skin summary (amount TBHC (μg) per surface area(cm²): mean of replicates ± SD;
amount TBHC (μg) per weight tissue (g): mean of replicates ± SD); % of the total applied dose).

| | | | | 0.1% TBHC/0.3% | | | 0.1% TBHC/0.3% | | |
|---|---|---|---|---|---|---|---|---|---|
| | Lamisil^{AT} Cream | | | Nanoemulsion a | | | Nanoemulsion b | | |
| | | μg/gram | % applied | | μg/gram | % applied | | | % applied |
| | μg/cm² | tissue | dose | μg/cm² | tissue | dose | μg/cm² | μg/g | dose |
| Epidermis | 4.3 ± 1.0 | 108.8 ± 37.9 | 0.21 ± 0.05 | 104.6 ± 36.0 | 2028.2 ± 919.6 | 5.23 ± 1.80 | 78.9 ± 31.2 | 1631.3 ± 596.9 | 3.94 ± 1.56 |
| Dermis | 2.0 ± 0.9 | 9.3 ± 3.5 | 0.10 ± 0.04 | 24.9 ± 7.1 | 102.6 ± 29.1 | 1.24 ± 0.36 | 47.2 ± 5.8 | 192.1 ± 16.7 | 2.36 ± 0.29 |
| Receptor | 0 | 0 | 0 | 0 | 0 | 0 | 0 | 0 | 0 |

Lamisil^{AT} cream delivered ˜12× times more TBHC into the epidermis as compared to the dermis. 1% TBHC/0.3% nanoemulsion a delivered ˜9× times more TBHC into the epidermis as compared to the dermis. 1% TBHC/0.3% nanoemulsion b delivered ˜20× times more TBHC into the epidermis as compared to the dermis.

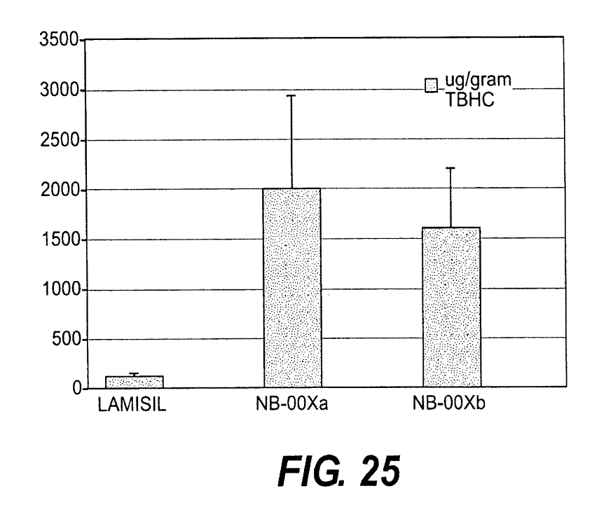

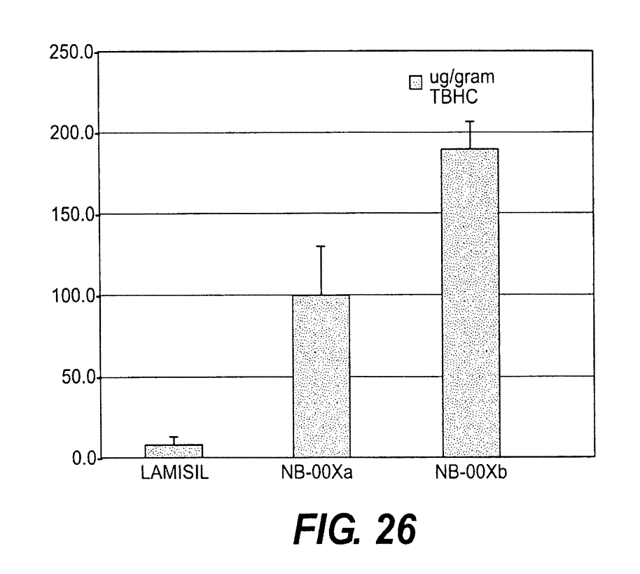

Absorption into the epidermis and dermis were measured 24 hours after two applications, at 0 hour and 8 hours, onto pig skin. There was an increase in the delivery of the TBHC into the epidermis (FIG. 25) and dermis (FIG. 26) with the 1% TBHC/0.3% nanoemulsion formulations as compared to the Lamisil^{AT} Cream formulation. The levels of TBHC found in the epidermis and dermis after 24 hour duration were lower for the Lamisil^{AT} formulation as compared to the 1% TBHC/0.3% nanoemulsions. The levels of TBHC in the epidermis were 18.6 and 15.1 times higher for 1% TBHC/0.3% nanoemulsion a and 1% TBHC/0.3% nanoemulsion b, respectively, as compared to the Lamisil^{AT} Cream formulation. The levels of TBHC in the dermis were 10.9 and 20 times higher for 1% TBHC/0.3% nanoemulsion a and 1% TBHC/0.3% nanoemulsion b, respectively, as compared to the Lamisil^{AT} Cream formulation. This indicates that superior delivery of TBHC into the skin was achieved after a topical application of the novel nanoemulsions containing TBHC. Thus, thee nanoemulsions significantly enhanced the TB delivery into the epidermis and dermis.

As demonstrated in FIGS. 25 and 26, the Lamisil® cream exhibited absorption of only about 0.2% of the total dose into the epidermis and about 0.1% of the total dose into the dermis. In contrast, the nanoemulsion formulations #1 exhibited absorption of about 3.7% and about 1.2% of the total dose into the epidermis and the dermis, respectively. Similarly, the nanoemulsion formulations #2 exhibited absorption of about 5.2% and about 2.7% of the total dose into the epidermis and the dermis, respectively, which are significant increases relative to the control Lamisil® cream.

### Example 6

The purpose of this example was to determine whether an active agent incorporated into a nanoemulsion formulation, such as terbinafine hydrochloride (TBHC), can diffuse laterally into human cadaver skin.

1% TBHC and 0.3% cetylpyridinium chloride (CPC) were incorporated into the NB-00Xb formulation. The oil-in-water nanoemulsions used in this study have a mean droplet diameters of approximately 180 nm. CPC resides at the interface between the oil and water phases. Lamisil® cream containing 1% TBHC was used as a control.

In vitro studies were carried out using excised human cadaver skin in a modified Franz diffusion apparatus. 1% TBHC/0.3% CPC NB-00Xb at 100 μL/cm² were applied to a 5.27 cm² concentric surface area of skin enclosed by two concentric glass cylinders. Twenty-four hours post application, residual nanoemulsion was removed by swabbing the dosing area. The epidermis and dermis of the dosing area was separated, weighed and assayed for CPC and TBHC. An 8 mm (0.5 cm² surface area) punch biopsy of the inner non-dosing area (inner area) and middle non-dosing area (middle area) were processed in similar fashion. Quantification of CPC and TBHC was performed by high pressure liquid chromatography (HPLC) with independent methods. The only way CPC or TBHC could be detected in the middle or inner tissues is through permeation of nanoemulsion into the skin underlying the dosing area followed by lateral diffusion into the non-dosing areas.

### 6.1 EXPERIMENTAL

### Test Formulations

Preparation of 1% TBHC/0.3% NB-00Xb

The nanoemulsion formulation of this study comprised: 0.3% CPC (0.3% NB-001 or 3 mg CPC/ml) and 1% TBHC. TBHC was incorporated into 1% NB-00Xb (containing 1% CPC) by first dissolving the TBHC in ethanol and then mixing with water. This solution was slowly added, with gentle mixing, to the 1% nanoemulsion to obtain a final product comprising 0.3% nanoemulsion with 1% TBHC. The final formulation comprised 22% ethanol and 57% water. The compositions of the TBHC nanoemulsion is shown in Table 23.

TABLE 23
Composition of the nanoemulsion (NB-00b); The percentages are wt/wt,
unless otherwise noted.

| | Soybean | Tween 20 | Ethanol | CPC | TBHC | EDTA | Water |
|---|---|---|---|---|---|---|---|
| Formulation | oil (%) | (%) | (%) | (% w/v) | (% w/v) | (%) | (%) |
| 1% TBHC/ | 18.837 | 1.776 | 22.037 | 0.320 | 1.0 | 0.022 | 56.01 |
| 0.3% NB-00Xb | | | | | | | |

Lamisil® was purchased from a local drug store and comprised 1% TBHC.

The test formulations were applied to the epidermal surface of the donor chamber of the diffusion cells using a positive displacement pipette. For single dosing, 527 μL was applied (e.g. QD). For multiple dosing (e.g. BID), 527 μL was applied 8 hours after the initial dosing. The exposed dosing epidermal surface area was 5.27 cm².

Human Cadaver Skin

Human cadaver back abdominal from a 75-year-old Caucasian male donor obtained from Life Legacy tissue bank was used in this study. The skin was cut into circular discs having 38 mm in diameter and the weights of the epidermis and dermis were recorded for each cell and from each dosing area and each non-dosing area before tissue extraction. The 1% TBHC/0.3% NB-00Xb formulation and Lamisil® were applied twice at 0 and 8 hours after the start of the study.

Modified Diffusion Apparatus

Percutaneous absorption was measured using the in vitro cadaver skin finite dose technique as described by Franz T J, “The finite dose technique as a valid in vitro model for the study of percutaneous absorption in man.,” Skin: Drug Application and Evaluation of Environmental Hazards, Current Problems in Dermatology, vol. 7, G. Simon, Z. Paster, M Klingberg, M. Kaye (Eds), Basel, Switzerland, S. Karger, 1978, pp 58-68.

Cryopreserved, dermatomed human cadaver trunk skin was obtained from Life Legacy organ donor bank and stored in aluminum foil pouches at −70° C. until use. At time of use, the skin was thawed by placing the sealed pouch in 37° C. water for approximately five minutes. The skin was removed from the pouch and cut into sections to fit on 38 mm permeation well cells. The receptor compartment was filled with 50 mL of distilled water, pH 7, and the donor compartment was left open to ambient laboratory conditions. All cells were mounted in a diffusion apparatus in which the receptor solution was maintained at 37° C. by a circulating water bath on the outside of the wells. The parameters for the diffusion study are listed in Table 24.

TABLE 24
Parameters for the Lateral Diffusion Methodology.

| | |
|---|---|
| Apparatus | Modified diffusion cell apparatus |
| Membrane | Human Cadaver Skin (75 yr old Male), |
| | Abdominal Skin) |
| Duration | 24 hours |
| Dosing Surface Area | Outer dosing area, 5.27 cm² |
| Non-dosing Surface Area | Inner non-dosing area, 0.5 cm² |
| | Middle non-dosing area, 3.3 cm² |
| Dose | 113 μL |
| Dose per Surface Area | 100 μL/cm² |
| Concentration | Lamisil ® (Lot# 10047765) |
| | 1% TBHC/0.3% NB-00Xb (Lot #89-59-02) |
| Dosing Frequency | QD: Once (0 hr); BID: Twice (0 and 8 hr) |
| Cell Volume | 50 mL |
| Receptor Solution | Distilled water, pH 7 |
| Sampling Volume | 1 mL |
| Receptor Sampling | 24 hours |
| Assay Method | HPLC |
| Samples Collected | Surface wash, epidermis, dermis, and |
| | receptor samples |

Two circular glass chambers were attached onto the skin surface using cyanoacrylate adhesive (e.g. super glue) as shown in FIG. 17. FIG. 16 illustrates the dimensions of the surface areas involved in the study. The test formulations were applied to the outer dosing area 15 minutes after modified Franz cell apparatus was prepared with skin samples. The middle and inner areas did not receive a topical application of the test formulations.

Sampling

Twenty-four hours after application of the first dose, the surface of the outer dosing area and the inner and middle areas were swabbed independently for several times with 70% ethanol solution to remove all residual formulation from the skin surface. All surface swabs were assayed for CPC content. 1 mL of the receptor solution was also sampled at 24 hours from the receptor of each cell and filtered through a 0.45 μm PTFE (25 mm) membrane syringe filter. The filtrates were collected in HPLC snap cap vials.

Skin samples were collected after removal of the glass chambers. First, the outer dosing area was processed. Briefly, the epidermis was removed from the dermis in the outer dosing area via a scraping technique, placed in a tared 20 mL glass vial and weighed. The dermis was then removed from the dosing area using a scalpel and placed in a tared 20 mL glass vial; weights were recorded. The middle and inner areas were processed in the same fashion. The epidermal and dermal tissues from the outer, middle and inner areas were extracted with 70% ethanol solution, sonicated for 1 hour, filtered through a 25 mm, 0.45 μm PTFE membrane syringe filter into HPLC vials and assayed using HPLC.

Analysis of Samples

Assay of terbinafine extracted from human cadaver skin samples was performed using a ten minute HPLC isocratic reversed-phase method using a Phenomenex Aqua C18 (150×4.6 mm, 5 μm) column at 40° C., 0.1% phosphoric acid in 40:60 Acetonitrile:water as mobile phase, and UV detection at 224 nm developed by Velesco, Ann Arbor Mich. The method was validated for linearity, precision, limit of quantitation, limit of detection and for specificity of terbinafine from skin epidermis, dermis, extraction solvent, and formulation excipients. Experimental conditions are tabulated below in Table 25.

TABLE 25
Experimental Conditions for HPLC Analysis of TBHC samples.

| | |
|---|---|
| HPLC System | Agilent 1100 HPLC System: |
| | Agilent Chemstation software Rev. B.03.02 [341] |
| | Pump Model# G1311A, |
| | Degasser Model# G1322A, |
| | Autosampler Model# G1329A, |
| | UV-VIS Detector Model# G1315B, |
| | Column Oven Model# G1316A |
| Mobile Phase | 0.1% Phosphoric Acid in 40:60 Acetonitrile:water |
| Column | Phenomenex Aqua C18, 4.6 mm diameter × |
| | 150 mm length, 5 μm particle size |
| TBHC Standard: | 5 ng/mL |
| Column Temperature | 40° C. |
| Injection Volume | 25 μL |
| Run Time | 10 minutes |
| Bracketing Standard | 5 μg/mL |

Assay of cetylpyridinium chloride extracted from human skin samples used a 12 minute isocratic reversed-phase method with a CPS-2 Hypersil (4.6 mm diameter×150 mm length, 5 μm particle size) column at 40° C., 45/55 (v/v %) Buffer (CTAB-KH₂PO₄), pH 2.5: methanol as the mobile phase, and UV detection at 260 nm. The method was validated for linearity, precision, limit of quantitation, limit of detection and for cetylpyridinium chloride specificity from skin epidermis, dermis, extraction solvent, and formulation excipients. Experimental conditions are tabulated below in Table 26.

TABLE 26
Experimental Conditions for HPLC Analysis of CPC samples.

| | |
|---|---|
| HPLC System | Shimadzu HPLC System: |
| | Liquid Chromatograph Model# LC-10ATVP |
| | System Controlled Model# SCL-10AVP, |
| | Degasser Model# DGU-14A, |
| | Autosampler Model# SIL-20ACVP, |
| | UV-VIS Detector Model# SPD-10VP, |
| | Column Oven Model# CTO-20AC |
| Mobile Phase | 45/55 (v/v %) Buffer (CTAB-KH₂PO₄), |
| | pH 2.5: Methanol |
| Column | CPS-2 Hypersil, 4.6 mm diameter × 150 mm length, |
| | 5 μm particle size |
| Column Temp. | 40° C. |
| Injection Volume | 100 μL |
| Run Time | 12 minutes |
| Bracketing Standard | 50 μg/mL |

Epidermal and Dermal Calculations

The amount of TBHC and CPC that permeated into the epidermis, dermis and the receptor compartment (at 24 hours after first dose) was determined by HPLC. A standard concentration of TBHC or CPC was generated and used to determine the concentration of TBHC or CPC in the dosing area. The levels of CPC or TBHC in each skin area are represented as: 1) amount per wet tissue weight (μg/grams)±the standard deviation; 2) amount per surface area (μg/cm²)±the standard deviation. The number of replicas used in the calculation was 3 or 4 for each formulation.

### 6.2 RESULTS

### TBHC Levels following Topical Administration

The results of permeation studies of Lamisil® and 1% TBHC/0.3% NB-00Xb for epidermal human cadaver skin and for dermal human cadaver skin are shown in Tables 27 and 28, respectively. The levels of TBHC delivered from NB-00Xb found in the various compartments (epidermis and dermis) were significantly different from levels of TBHC delivered from Lamisil® cream. The levels of TBHC found in the epidermis and dermis after 24 hour duration were lower for the Lamisil® cream as compared to the 1% TBHC/0.3% NB-00Xb formulation.

The levels of TBHC found in the outer, middle and inner epidermis of the samples treated by the NB-00Xb formulations were 14, 35 and 310 times higher (μg/g tissue levels), respectively, relative to the same areas (outer, middle inner) of the samples treated by the Lamisil® cream. The levels of TBHC found in the outer, middle and inner epidermis of the samples treated by the 1% TBHC/0.3% NB-00Xb formulation were 27, 28 and 115 times higher (μg/g tissue levels), respectively, relative to the same areas (outer, middle, inner) of the samples treated by the Lamisil® cream. Also, the amount of TBHC found in the surface swabs of the middle and inner surface areas at 24 hours was below detection level of 5 μg/ml for all the formulations, indicating no leakage of the test article from the dosing area to non-dosing areas.

TABLE 27
Epidermal human cadaver skin summary: percutaneous absorption of TBHC formulations
through human cadaver skin over 24 hours from BID topical dosing (0 and 8 hrs).

| | Lamisil ® Cream, BID | | 1% TBHC/0.3% CPC NB-00Xb, BID | |
|---|---|---|---|---|
| | TBHC | TBHC μg/g | TBHC | TBHC (μg/g |
| Parameter | (μg/cm²) | wet tissue | (μg/cm²) | wet tissue) |
| Outer Dosing Area | 2.05 ± 0.92 | 193.8 ± 77.0 | 35.23 ± 15.4 | 2788.0 ± 810.7 |
| Middle Area | 0.21 ± 0.23 | 48.2 ± 49.8 | 9.87 ± 5.69 | 1686.3 ± 1175.9 |
| Inner Area | 0.013 ± 0.023 | 2.12 ± 3.73 | 4.30 ± 2.10 | 621.0 ± 330.3 |
| Number of Replica | 3 | 3 | 4 | 4 |

TABLE 28
Dermal human cadaver skin summary: percutaneous absorption of TBHC formulations
through human cadaver skin over 24 hours from BID topical dosing (0 and 8 hrs).

| | Lamisil ® Cream, BID | | 0.3% CPC/1% TBHC in NB-00Xb, BID | |
|---|---|---|---|---|
| | TBHC | TBHC μg/g | TBHC | TBHC (μg/g |
| Parameter | μg/cm²) | wet tissue | (μg/cm²) | wet tissue) |
| Outer Dosing Area | 0.59 ± 0.35 | 6.8 ± 6.1 | 18.9 ± 4.1 | 182.1 ± 46.0 |
| Middle Area | 0.16 ± 0.14 | 3.59 ± 3.93 | 6.95 ± 6.59 | 96.8 ± 53.8 |
| Inner Area | 0.01 ± 0.02 | 2.15 ± 3.73 | 2.22 ± 1.81 | 248.3 ± 242.2 |
| Number of Replica | 3 | 3 | 4 | 4 |

### 6.3 CONCLUSIONS

The lateral diffusion data of nanoemulsions comprising terbinafine hydrochloride indicate that the nanoemulsions traversed laterally under the stratum corneum to tissues for up to 11 mm away from the dosing area.

### Example 7

### In Vitro Permeation Studies for Nanoemulsion Formulations Comprising Miconazole and Lotrimin® Spray Solution Containing Miconazole Nitrate

The purpose of this example was to determine whether an active agent incorporated into a nanoemulsion formulation, such as terbinafine hydrochloride (TBHC), can diffuse laterally into skin. In particular, this example investigated the potential of nanoemulsion formulations to deliver miconazole (MCZ) into swine skin. Commercially available Lotrimin AF®, Spray Solution was used as a control. Cetylpyridinium chloride (CPC), a cationic surfactant in the nanoemulsion, was used as an additional marker agent of delivery for the nanoemulsion.

Miconazole is an imidazole antifungal agent commonly applied topically to the skin or mucus membranes to cure fungal infections. It works by inhibiting the synthesis of ergosterol, a critical component of fungal cell membranes. It can also be used against certain species of Leishmania protozoa, which are a type of unicellular parasite, as these also contain ergosterol in their cell membranes. In addition to its antifungal and antiparasitic actions, it also has some limited antibacterial properties. Miconazole is mainly used externally for the treatment of athlete's foot, ringworm and jock itch. Internal application is used for oral or vaginal thrush (yeast infection). In addition, the oral gel may also be used for the lip disorder angular cheilitis. The chemical structure and physical chemical properties are given below.

### Chemical Structure of Miconazole:

TABLE 29

| 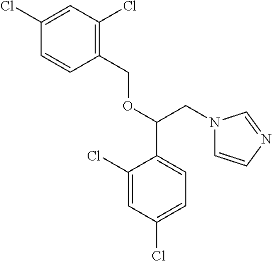 | |
|---|---|
| Physical-chemical properties of miconazole. | |
| CAS Number | 22916-47-8 |
| Molecular Formula | C₁₈H₁₄Cl₄N₂O |
| Molar Mass | 416.13 g/mol |
| Melting Point | 170.5 ° C. |
| Log P/pKa | 6.1/ 6.67 |
| Water Solubility | 0.03% |
| Soybean Solubility | 74 mg/ml |
| Ethanol Solubility | 94 mg/ml |

### 7.1. Test Formulations

Preparation of 2% Miconazole/0.3% Nanoemulsion

The nanoemulsion test formulations comprised a final concentration of 0.3% (0.3% CPC or 3 mg CPC/ml) and 2% miconazole. Miconazole was incorporated into a 1% nanoemulsion (comprising 1% CPC) by first dissolving the miconazole in ethanol until completely solubilized and then mixing with the water. This solution was slowly added, with gentle mixing, to the 1% nanoemulsion to obtain a final product containing 0.3% nanoemulsion with 2% miconazole. No evidence of miconazole precipitation was observed after mixing with the nanoemulsion by visual inspection and microscopy. Miconazole can also be soluiblized in the oil phase prior to emulsion formulation. The composition of the miconazole nanoemulsion is listed in Table 30.

TABLE 30
Composition of the Nanoemulsion (MCZ/NB-00X). The percentages are
wt/wt, unless otherwise noted.

| | | Soybean | Tween | Ethanol | CPC | MCZ % | EDTA | Water |
|---|---|---|---|---|---|---|---|---|
| Formulation | Lot # | oil % | 20 % | % | % | (wt/v) | % | % |
| 2% MCZ/0.3% | 89-59-03 | 18.837 | 1.776 | 12.037 | 0.320 | 2.0 | 0.022 | 66.01 |
| NB-00X | | | | | | | | |

Lotrimin AF®, Spray Solution contained 2% miconazole nitrate. Inactive ingredients in Lotrimin AF®, Spray Solution include denatured alcohol (13% v/v), cocamide DEA, isobutene, propylene glycol and tocopherol (vitamin E).

### 7.2. Epidermal and Dermal Calculations

The amount of MCZ that permeated into the epidermis, dermis and the receptor compartment (at 24 hours after first dose) was determined by HPLC MS/MS. A standard concentration of MCZ was generated and used to determine the concentration of MCZ in the dosing area. The levels of CPC or MCZ in each skin area are represented as: (1) amount per surface area (μg/cm²)±the standard deviation; (2) amount per wet tissue weight (μg/grams)±the standard deviation; (3) the % of the applied dose±the standard deviation. The number of replicas used in the calculation was 5 for each formulation.

In vitro skin permeation studies were performed using a diffusion cell methodology, as described in FIGS. 16 and 17. Twenty-four hours after two applications of the nanoemulsion formulation and Lotrimin AF®, Spray Solution, the epidermis and dermis were separated, weighed and assayed for miconazole by LC/MS/MS. Samples from the receptor were also assayed for miconazole. In the nanoemulsion formulations containing miconazole, CPC concentrations were also determined by HPLC.

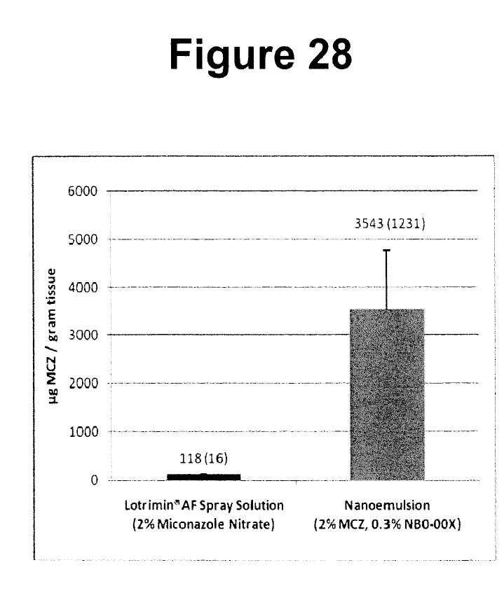

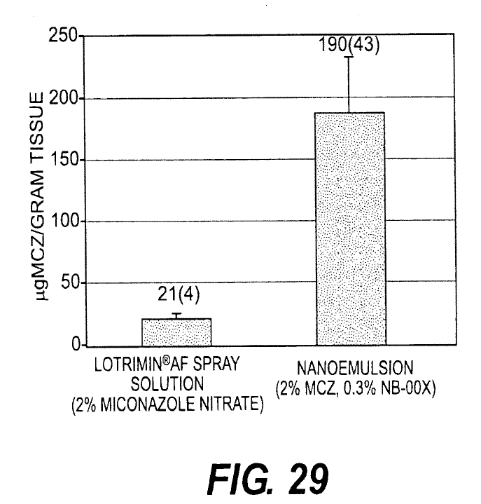

The results of MCZ permeation studies for Lotrimin®j Spray Solution and 2% MCZ/0.3% nanoemulsion are shown in Table 31 and FIGS. 28 and 29.

TABLE 31
Percutaneous absorption of MCZ formulations into swine skin over 24 hours from BID dosing.
Epidermal and dermal pig skin summary (amount MCZ (μg) per surface area (cm²): mean of
replicates ± SD; amount MCZ (μg) per weight tissue (g): mean of replicates ± SD);
% of the total applied dose).

| | Lotrimin ®_{AF} Spray Solution | | | 2% MCZ/0.3% NB-00X | | |
|---|---|---|---|---|---|---|
| | | MCZ | % | | MCZ | |
| | MCZ | μg/gram | applied | MCZ | μg/gram | % applied |
| | μg/cm² | tissue | dose | μg/cm² | tissue | dose |
| Epidermis | 6.54 ± 2.29 | 118.4 ± 16.2 | 0.16 ± 0.05 | 153.8 ± 43.1 | 3543.5 ± 1213.2 | 3.84 ± 1.08 |
| Dermis | 4.6 ± 0.8 | 21.2 ± 4.0 | 0.11 ± 0.02 | 41.6 ± 10.2 | 190.9 ± 43.5 | 1.04 ± 0.25 |
| Receptor | 0 | 0 | 0 | 0 | 0 | 0 |

Commercially available Lotrimin® Spray Solution delivered ˜5.6× times more MCZ into the epidermis as compared to the dermis. Surprisingly, the nanoemulsion formulation comprising 2% MCZ/0.3% NB-00X delivered ˜18.6× times more MCZ into the epidermis as compared to the dermis. Thus, there was a significant increase in the delivery of the MCZ into the epidermis and dermis with the 2% MCZ/0.3% nanoemulsion formulation as compared to the Lotrimin AF®, Spray Solution. The levels of MCZ found in the epidermis and dermis after 24 hours were lower for the Lotrimin Spray formulation compared to the 2% MCZ/0.3% nanoemulsion formulation. The levels of MCZ in the epidermis were 30 times higher for 2% MCZ/0.3% nanoemulsion as compared to the Lotrimin AF®, Spray Solution. The levels of MCZ in the dermis were 9 times higher for 2% MCZ/0.3% nanoemulsion as compared to the Lotrimin AF®, Spray Solution. Thus, there is increased delivery of MCZ into epidermal and dermal tissues using the nanoemulsion formulation as compared to the Lotrimin AF®, Spray Solution. The amount of MCZ found in the receptor compartment at 24 hours was below the level of detection (50 ng/ml) for all formulations tested.

### Example 8

The purpose of this example was to determine the virucidal activity of a nanoemulsion according to the invention.

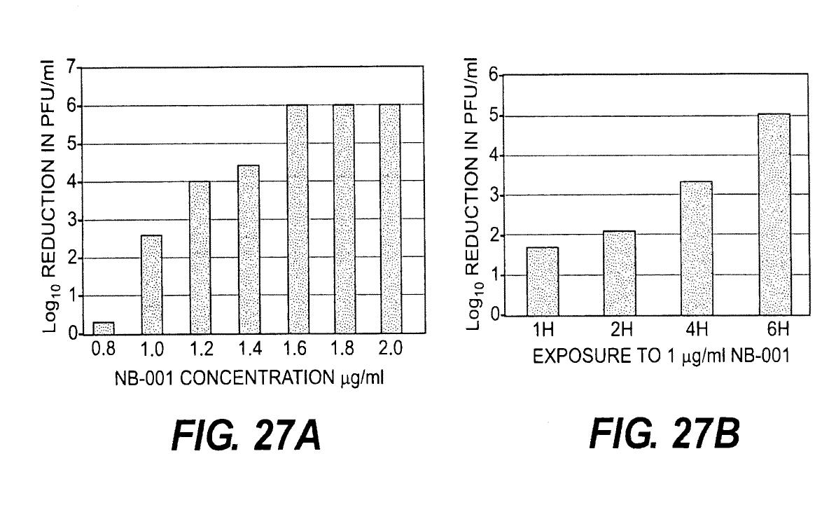

The in vitro virucidal activity of a nanoemulsion according to the invention is dependent upon nanoemulsion concentration, duration of exposure, and viral load. FIG. 27A shows that maximal reduction in viral load is reached within 15 minutes at concentrations of 1.6 μg/mL (0.00016% NB-001). To achieve a ≧3-log kill of HSV-1 KOS strain at concentrations near the IC₅₀ (0.0001% NB-001 or 1 μg CPC/mL) and a viral load of 2.7×10⁷ pfu/mL requires 4 hours incubation at room temperature (22° C.; FIG. 27 B).

NB-001 has been tested against other herpes simplex virus strains. Assessment of NB-001's activity to kill the virus in suspension was performed using the ASTM E1052-96 method (American Society for Testing and Materials (ASTM E1052-96, 2002) because it does not require the virus to be actively replicating to exert its antiviral activity. NB-001 was equally virucidal against HSV-1 and HSV-2 strains, with a range of IC₅₀ values of 0.5-4.3 μg/mL (FIGS. 11 and 12). There was no cross-resistance to NB-001 when mutations conferring resistance to either the nucleoside analogue acyclovir (ACV) or the pyrophosphate analogue, foscarnet (FOS) were tested. Although HSV-2 strains are most commonly found in genital herpes, HSV-1 is the most common cause of newly diagnosed genital herpes in developed countries. The majority of acyclovir and/or foscarnet resistant strains occur in immunocompromised patients who use nucleoside analogues prophylactically to prevent recurrent outbreaks.

### Example 9

The purpose of this example was to evaluate the effect of crystallization of a nanoemulsion according to the invention as the concentration of the nanoemulsion is increased.

Aqueous formulations of CPC at 0.1%, 0.3% and 0.5% were compared with nanoemulsions containing 0.1%, 0.3% and 0.5% CPC following application to glass slides and viewing by cross polar light microscopy over time. The table below demonstrates the time dependent formation of crystals from 3% aqueous CPC (3 mg/mL), when applied to a glass slide. Crystallization is apparent within 10 minutes with essentially complete crystallization occurring within 30 minutes. If the same amount of CPC was formulated as a nanoemulsion (0.3% NB-001), then crystallization did not occur to any significant degree for 2 hours. 0.5% NB-001 showed extensive crystal formation within 30 minutes. CPC crystallized on the slide surface as water/ethanol evaporated.

TABLE 32
Visual assessment of crystal formation on glass slides
comparing aqueous CPC, 0.3% and 0.5% NB-001

| Time after | | Aqueous CPC | 0.3% NB-001 | 0.5% NB-001 |
|---|---|---|---|---|
| application | | (3 mg CPC/mL) | (3 mg CPC/mL) | (5 mg CPC/mL) |
| 0 | min | No crystals | No crystals | No crystals |
| 10 | min | A few small | No crystals | No crystals |
| | | crystals | | |
| 20 | min | Large crystals | No crystals | Small crystals |
| 30 | min | Many crystals | No crystals | Large crystals |
| 1 | hr | Many crystals | Amorphous | Large crystals |
| | | | structures | |
| 1.5 | hr | Many crystals | Amorphous + | Same as 1 hour |
| | | | some crystals | |
| 2 | hr | —^{a} | Large crystals + | Same as 1 hour |
| | | | some crystals | |
| 4 | hr | — | Same as 2 hours | Same as 1 hour |
| 5 | hr | — | Same as 2 hours | Same as 1 hour |

^{a}Last record of crystallization at 1.5 hr.

9.1 Permeation of NB-001 into Skin

Human cadaver skin was used as an in vitro model to study the permeation of NB-001 into the epidermis and dermis. 24 hours after a single application of 0.3% NB-001 (3 mg CPC/mL), 2.4 mg CPC/gm epidermal tissue was recovered while 27 μg CPC/gm of tissue was delivered to the dermis. Thus, the concentration of drug in both the epidermis and dermis exceeded the IC₅₀ for representative viruses. (Data not shown.) In contrast, when a 3 mg/mL aqueous solution of CPC was applied to human cadaver skin, minimal or no levels of CPC were found in either the epidermis or dermis. (Data not shown.) This is attributable to crystallization of CPC from the solution that occurs at the skin surface due to rapid evaporation of water, so that very little, if any, CPC is delivered into the skin despite micelles being small enough to penetrate the stratum corneum.

Five applications of 0.1, 0.3 or 0.5% NB-001 (each application was 1134 applied over a dosing area of 1.13 cm²) were made to human cadaver skin over 12 hours to reflect the delivery of NB-001 and delivery was determined at 24 hours by measuring CPC concentration in the epidermis and dermis (FIGS. 14 and 15). 0.3% NB-001 delivered the highest concentration of CPC achieving 10-fold higher levels in the epidermis (2.6 mg CPC/g of tissue) and 4-fold higher levels in the dermis (20.3 μg CPC/g of tissue) than 0.1% or 0.5% NB-001. Delivery of CPC from 0.5% NB-001 into the epidermis and dermis was significantly reduced compared to 0.3% NB-001 and was due to evaporation of the ethanol/water components of the nanoemulsion resulting in crystallization of CPC from the nanoemulsion after application to the skin. This can also be visualized using cross-polar light microscopy which shows the presence of crystals on human cadaver skin after 0.5% NB-001 was applied.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A method of treating a herpes virus infection, preventing a herpes virus infection, preventing recurrent herpes virus infection, preventing reactivation of a herpes virus, minimizing reactivation of a herpes virus, or a combination thereof, in a human subject in need thereof comprising topically or intradermally administering to the human subject a nanoemulsion, wherein:
(a) the topical application is to the herpes lesion, the skin surrounding the herpes lesion, or a combination thereof
(b) the nanoemulsion comprises droplets having an average diameter of less than about 1000 nm, and
(c) the nanoemulsion comprises water, at least one oil, at least one surfactant, and at least one organic solvent,
wherein the method results in a reduced time to heal as compared to treatment with vehicle, no treatment, or a treatment with a non-nanoemulsion composition.

2. The method of claim 1, wherein:
(a) the nanoemulsion kills, weakens, disables or reduces pathogenicity of the herpes virus;
(b) the nanoemulsion is preventative against the herpes infection, recurrent infection, or reactivation of virus; or
(c) a combination thereof.

3. The method of claim 1, wherein:
(a) the nanoemulsion is therapeutically effective against the herpes virus;
(b) the nanoemulsion is virucidal or virustatic against the herpes virus;
(c) following treatment, partial or complete clearing of lesions is observed;
(d) the nanoemulsion prevents lesions from appearing or developing;
(e) the nanoemulsion reduces the time to healing when the baseline is the prodrome lesion stage;
(f) the nanoemulsion reduces the time to healing when the baseline is the erythema lesion stage;
(g) the nanoemulsion reduces the time to healing when the baseline is the papule lesion stage;
(h) the nanoemulsion reduces the time to healing when the baseline is the vesicle lesion stage; or
(i) any combination thereof.

4. The method of claim 1, wherein:
(a) the nanoemulsion droplets associate with the virus resulting in death, growth inhibition, a loss of pathogenicity, or any combination thereof;
(b) the nanoemulsion droplets heal, prevent, or inhibit the onset of lesions; or
(c) any combination thereof.

5. The method of claim 1, wherein the nanoemulsion is applied to the orofacial region, the eye, the urogenital region (external or internal, skin or mucosa), vaginal mucosa, rectal mucosa, anal mucosa, oral mucosa, extremities, skin, oral pharynx, superficial skin structure and appendages, lips, vermillion border, all areas of the mouth, neck, perineum, upper legs, hand, cornea, urethra, or any combination thereof.

6. The method of claim 1, wherein the herpes infection is caused by a herpes virus selected from the group consisting of Herpes Simplex Virus Type 1 (HSV-1), Herpes Simplex Virus Type 2 (HSV-2), Varicella Zoster Virus (VZV), Epstein-Bar Virus (EBV), Cytomegalovirus (CMV), Herpes Lymphotropic Virus, Human Herpes Virus Type 7 (HHV-7), Human Herpes Virus Type 8 (HHV-8), and a combination thereof.

7. The method of claim 1, wherein: (a) the method is used to treat a subject having resistance to one or more antiviral agents; (b) the subject has resistance to nucleoside analogs and/or foscarnet (c) the subject is resistant to acyclovir; or (d) any combination thereof.

8. The method of claim 1, wherein:
(a) the herpes infection is latent, active, or reactivated;
(b) the herpes is latent in the trigeminal ganglion, B lymphocyte, lumbrosacral ganglia, monocytes, neuron, T lymphocyte, or epithelial cells; or
(c) a combination thereof.

9. The method of claim 1, wherein the herpes infection is herpes labialis, genital herpes, ocular herpes, herpes rugbiorum, herpes gladiatorum, or herpetic whitlow.

10. The method of claim 1, wherein the nanoemulsion droplets traverse and/or diffuse through hair follicles, skin pores, mucosa, cornea, compromised skin, the epidermis, dermis, skin, scalp, damaged skin, diseased skin, or any combination thereof.

11. The method of claim 1, wherein the nanoemulsion droplets have an average diameter selected from the group consisting of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, greater than about 50 nm, greater than about 70 nm, greater than about 125 nm, and any combination thereof.

12. The method of claim 1, wherein the nanoemulsion further comprises:
(a) a chelating agent;
(b) a silicone component;
(c) at least one preservative;
(d) a pH adjuster;
(e) a buffer;
(f) another active agent; or
(g) any combination thereof.

13. The method of claim 12, wherein:
(a) the chelating agent is present in amount of about 0.0005% to about 1%; or the chelating agent is selected from the group consisting of ethylenediamine, ethylenediaminetetraacetic acid, and dimercaprol; or a combination thereof;
(b) the silicone component:
(i) comprises at least one volatile silicone oil, wherein the volatile silicone oil can be the sole oil in the silicone component or it can be combined with other silicone and non-silicone oils, and wherein the other oils can be volatile or non-volatile;
(ii) is selected from the group consisting of methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof; or
(iii) any combination thereof.
(c) the preservative is selected from the group consisting of cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens). semi-synthetic derivatives thereof, and combinations thereof;
(d) the pH adjuster is selected from the group consisting of diethyanolamine, lactic acid, monoethanolamine, triethylanolamine, sodium hydroxide, sodium phosphate, semi-synthetic derivatives thereof, and combinations thereof;
(e) the buffer is selected from the group consisting of 2-Amino-2-methyl-1,3-propanediol, 2-Amino-2-methyl-1-propanol, L-(+)-Tartaric acid, ACES, ADA, Acetic acid, Ammonium acetate solution, Ammonium bicarbonate, Ammonium citrate dibasic, Ammonium formate, Ammonium oxalate monohydrate, Ammonium phosphate dibasic, Ammonium phosphate monobasic, Ammonium sodium phosphate dibasic tetrahydrate, Ammonium sulfate solution, Ammonium tartrate dibasic, BES buffered saline, BES, BICINE, BIS-TRIS, Bicarbonate buffer solution, Boric acid, CAPS, CHES, Calcium acetate hydrate, Calcium carbonate, Calcium citrate tribasic tetrahydrate, Citrate Concentrated Solution, Citric acid, hydrous, Diethanolamine, EPPS, Ethylenediaminetetraacetic acid disodium salt dihydrate, Formic acid solution, Gly-Gly-Gly, Gly-Gly, Glycine, HEPES, Imidazole, Lipoprotein Refolding Buffer, Lithium acetate dihydrate, Lithium citrate tribasic tetrahydrate, MES hydrate, MES monohydrate, MES solution, MOPS, Magnesium acetate solution, Magnesium acetate tetrahydrate, Magnesium citrate tribasic nonahydrate, Magnesium formate solution, Magnesium phosphate dibasic trihydrate, Oxalic acid dihydrate, PIPES, Phosphate buffered saline, piperazine, Potassium D-tartrate monobasic, Potassium acetate, Potassium bicarbonate, Potassium carbonate, Potassium chloride, Potassium citrate monobasic, Potassium citrate tribasic solution, Potassium formate, Potassium oxalate monohydrate, Potassium phosphate dibasic, Potassium phosphate dibasic, for molecular biology, anhydrous, Potassium phosphate monobasic, Potassium phosphate monobasic, Potassium phosphate tribasic monohydrate, Potassium phthalate monobasic, Potassium sodium tartrate, Potassium sodium tartrate tetrahydrate, Potassium tetraborate tetrahydrate, Potassium tetraoxalate dihydrate, Propionic acid, STE buffer, STET buffer, Sodium 5,5-diethylbarbiturate, Sodium acetate, Sodium acetate trihydrate, Sodium bicarbonate, Sodium bitartrate monohydrate, Sodium carbonate decahydrate, Sodium carbonate, Sodium citrate monobasic, Sodium citrate tribasic dihydrate, Sodium formate solution, Sodium oxalate, Sodium phosphate dibasic dihydrate, Sodium phosphate dibasic dodecahydrate, Sodium phosphate dibasic solution, Sodium phosphate monobasic dihydrate, Sodium phosphate monobasic monohydrate, Sodium phosphate monobasic solution, Sodium pyrophosphate dibasic, Sodium pyrophosphate tetrabasic decahydrate, Sodium tartrate dibasic dihydrate, Sodium tartrate dibasic solution, Sodium tetraborate decahydrate, TAPS, TES, TM buffer solution, TNT buffer solution, TRIS Glycine buffer, TRIS acetate—EDTA buffer solution, TRIS buffered saline, TRIS glycine SDS buffer solution, TRIS phosphate—EDTA buffer solution, Tricine, Triethanolamine, Triethylamine, Triethylammonium acetate buffer, Triethylammonium phosphate solution, Trimethylammonium acetate solution, Trimethylammonium phosphate solution, Tris-EDTA buffer solution, Trizma® acetate, Trizma® base, Trizma® carbonate, Trizma® hydrochloride, Trizma® maleate, or any combination thereof;
(f) the additional agent is an antiviral agent selected from the group consisting of nucleoside analogs (e.g., acyclovir (Zovirax®), famciclovir (Famvir®), and valaciclovir (Valtrex®)), amantadine (Symmetrel®), oseltamivir (Tamiflu®), rimantidine (Flumadine®), and zanamivir (Relenza®), Cidofovir (Vistide®), foscarnet (Foscavir®), ganciclovir (Cytovene®), ribavirin (Virazole®), penciclovir (Denavir®), buciclovir, acyclic guanosine derivatives, (E)-5-(2-bromovinyl)-2′-deoxyuridine and structurally related analogues thereof [i.e., the cytosine derivative (E)-5-(2-bromovinyl)-2′-deoxycytidine and the 4′-thio derivative (E)-5-(2-bromovinyl)-2′-deoxy-4′-thiouridine], Nucleoside/Nucleotide Analogues (e.g., Abacavir (Ziagen, ABC), Didanosine (Videx, ddI), Emtricitabine (Emtriva, FTC), Lamivudine (Epivir, 3TC), Stavudine (Zerit, d4T), Tenofovir (Viread, TDF), Zalcitabine (Hivid, ddC), and Zidovudine (Retrovir, AZT, ZDV)); Nonnucleoside Reverse Transcriptase Inhibitors (e.g., Delavirdine (Rescriptor, DLV), Efavirenz (Sustiva, Stocrin, EFV), Etravirine (Intelence, TMC 125), Nevirapine (Viramune, NVP)); Protease Inhibitors (Amprenavir (Agenerase, APV), Atazanavir (Reyataz, ATV), Darunavir (Prezista, DRV, TMC 114), Fosamprenavir (Lexiva, Telzir, FPV), Indinavir (Crixivan, IDV), Lopinavir/Ritonavir (Kaletra), Nelfinavir (Viracept, NFV), Ritonavir (Norvir, RTV), Saquinavir (Invirase, SQV), and Tipranavir (Aptivus, TPV)); Fusion Inhibitors (e.g., Enfuvirtide (Fuzeon, ENF, T-20)); Chemokine Coreceptor Antagonists (e.g., Maraviroc (Selzentry, Celsentri, MVC)); and Integrase Inhibitors (e.g., Raltegravir (Isentress, RAL)). Preferred antiviral agents for incorporation into a nanoemulsion include, but are not limited to, acyclovir (Zovirax®), famciclovir (Famvir®), and valacyclovir (Valtrex®);
(g) the additional agent is selected from the group consisting of menthol, camphor, phenol, allantoin, benzocaine, corticosteroids, phenol, zinc oxide, camphor, pramoxine, dimethicone, meradimate, octinoxate, octisalate, oxybenzone, dyclonine, alcohols, mineral oil, propylene glycol, titanium dioxide, magnesium stearate, and docosanol; or
(h) any combination thereof.

14. The method of claim 1, wherein the nanoemulsion comprises:
(a) an aqueous phase;
(b) about 1% oil to about 80% oil;
(c) about 0.1% organic solvent to about 50% organic solvent;
(d) at least one surfactant present in an amount of about 0.001% to about 10%;
(e) at least one chelating agent present in an amount of about 0.0005% to about 1%; or
(f) any combination thereof.

15. The method of claim 1, wherein the nanoemulsion comprises:
(a) an aqueous phase;
(b) about 5% oil to about 80% oil;
(c) about 0.1% organic solvent to about 10% organic solvent;
(d) at least one non-ionic surfactant present in an amount of about 0.1% to about 10%;
(e) at least one cationic agent present in an amount of about 0.01% to about 2%;
(f) at least one chelating agent present in an amount of about 0.0005% to about 1%; or
(g) any combination thereof.

16. The method of claim 1, wherein the nanoemulsion is stable:
(a) at about 40° C. and about 75% relative humidity for a time period selected from the group consisting of up to about 1 month, up to about 3 months, up to about 6 months, up to about 12 months, up to about 18 months, up to about 2 years, up to about 2.5 years, and up to about 3 years;
(b) at about 25° C. and about 60% relative humidity for a time period selected from the group consisting of up to about 1 month, up to about 3 months, up to about 6 months, up to about 12 months, up to about 18 months, up to about 2 years, up to about 2.5 years, up to about 3 years, up to about 3.5 years, up to about 4 years, up to about 4.5 years, and up to about 5 years; or
(c) at about 4° C. for a time period selected from the group consisting of up to about 1 month, up to about 3 months, up to about 6 months, up to about 12 months, up to about 18 months, up to about 2 years, up to about 2.5 years, up to about 3 years, up to about 3.5 years, up to about 4 years, up to about 4.5 years, up to about 5 years, up to about 5.5 years, up to about 6 years, up to about 6.5 years, and up to about 7 years.

17. The method of claim 1, wherein the organic solvent:
(a) is selected from the group consisting of a C₁-C₁₂ alcohol, diol, triol, dialkyl phosphate, tri-alkyl phosphate, and combinations thereof
(b) is selected from the group consisting of a nonpolar solvent, a polar solvent, a protic solvent, an aprotic solvent, semi-synthetic derivatives thereof, and combinations thereof
(c) is selected from the group consisting of tri-n-butyl phosphate, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof; and
(d) any combination thereof.

18. The method of claim 1, wherein the oil is:
(a) any cosmetically or pharmaceutically acceptable oil;
(b) non-volatile;
(c) selected from the group consisting of animal oil, vegetable oil, natural oil, synthetic oil, hydrocarbon oils, silicone oils, and semi-synthetic derivatives thereof;
(d) selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and combinations thereof; or
(d) any combination thereof.

19. The method of claim 1, wherein the nanoemulsion comprises a volatile oil and wherein:
(a) the volatile oil is the organic solvent;
(b) the volatile oil is present in addition to an organic solvent;
(c) the volatile oil used in a silicone component is different than the oil in the oil phase;
(d) the volatile oil is a terpene, monoterpene, sesquiterpene, carminative, azulene, semi-synthetic derivatives thereof, or combinations thereof;
(e) the volatile oil is selected from the group consisting of a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives thereof, and combinations thereof; or
(f) any a combination thereof.

20. The method of claim 1, wherein:
(a) the surfactant is a pharmaceutically acceptable ionic surfactant, pharmaceutically acceptable ionic polymeric surfactant, a pharmaceutically acceptable nonionic surfactant, a pharmaceutically acceptable nonionic polymeric surfactant, a pharmaceutically acceptable cationic surfactant, a pharmaceutically acceptable cationic polymeric surfactant, a pharmaceutically acceptable anionic surfactant, a pharmaceutically acceptable anionic polymeric surfactant, a pharmaceutically acceptable zwitterionic surfactant, or a pharmaceutically acceptable zwitterionic polymeric surfactant;
(b) the surfactant is a polymeric surfactant selected from the group consisting of a graft copolymer of a poly(methyl methacrylate) backbone with at least one polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, and combinations thereof or
(c) a combination thereof.

21. The method of claim 1, wherein:
(a) the surfactant is selected from the group consisting of ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, and mixtures thereof
(b) the surfactant is a non-ionic lipid selected from the group consisting of glyceryl laurate, glyceryl myristate, glyceryl dilaurate, glyceryl dimyristate, semi-synthetic derivatives thereof, and mixtures thereof
(c) the surfactant is a polyoxyethylene fatty ether having a polyoxyethylene head group ranging from about 2 to about 100 groups;
(d) the surfactant is an alkoxylated alcohol having the structure shown in formula I below:
R₅—(OCH₂CH₂)_{y}—OH Formula I
wherein R₅ is a branched or unbranched alkyl group having from about 6 to about 22 carbon atoms and y is between about 4 and about 100, and preferably, between about 10 and about 100;
(e) the surfactant is an alkoxylated alcohol according to(d), wherein R₅ is a lauryl group and y has an average value of 23;
(f) the surfactant is an alkoxylated alcohol which is an ethoxylated derivative of lanolin alcohol;
(g) e surfactant is an alkoxylated alcohol which is an ethoxylated derivative of lanolin alcohol, wherein the ethoxylated derivative of lanolin alcohol is laneth-10, which is the polyethylene glycol ether of lanolin alcohol with an average ethoxylation value of 10;
(h) the surfactant is nonionic and is selected from the group consisting of nonoxynol-9, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), Brij® 35, Brij® 56, Brij® 72, Brij® 76, Brij® 92V, Brij® 97, Brij® 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, Heptaethylene glycol monodecyl ether, Heptaethylene glycol monotetradecyl ether, Heptaethylene glycol monododecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Methyl-6-O-(N-heptylcarbamoyl)-alpha-D-glucopyranoside, Nonaethylene glycol monododecyl ether, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Tergitol Type 15-S-12, Tergitol Type 15-S-30, Tergitol Type 15-S-5, Tergitol Type 15-S-7, Tergitol Type 15-S-9, Tergitol Type NP-10, Tergitol Type NP-4, Tergitol Type NP-40, Tergitol Type NP-7, Tergitol Type NP-9, Tergitol Type TMN-10, Tergitol Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton X-114, Triton X-165, Triton X-305, Triton X-405, Triton X-45, Triton X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, Poloxamer 182, Dibenzoate, semi-synthetic derivatives thereof, and combinations thereof;
(i) the surfactant is cationic and is selected from the group consisting of a quarternary ammonium compound, an alkyl trimethyl ammonium chloride compound, a dialkyl dimethyl ammonium chloride compound, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Cetylpyridinium chloride, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl didecyl dimethyl ammonium chloride, Alkyl dimethyl benzyl ammonium chloride (C12-16), Alkyl dimethyl benzyl ammonium chloride (C12-18), dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C₈₋₁₀)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl) octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof;
(j) the surfactant is anionic and is selected from the group consisting of a carboxylate, a sulphate, a sulphonate, a phosphate, Chenodeoxycholic acid, Chenodeoxycholic acid sodium salt, Cholic acid, ox or sheep bile, Dehydrocholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-dodecanesulfonate, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, Ursodeoxycholic acid, semi-synthetic derivatives thereof, and combinations thereof;
(k) the surfactant is zwitterionic and is selected from the group consisting of an N-alkyl betaine, lauryl amindo propyl dimethyl betaine, an alkyl dimethyl glycinate, an N-alkyl amino propionate, CHAPS (minimum 98%), CHAPSO (minimum 98%), 3-(Decyldimethylammonio)propanesulfonate inner salt, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof; or
(l) any combination thereof.

22. The method of claim 1, wherein the nanoemulsion:
(a) comprises at least one cationic surfactant;
(b) comprises a cationic surfactant which is cetylpyridinium chloride;
(c) comprises a cationic surfactant, and wherein the concentration of the cationic surfactant is less than about 5.0% and greater than about 0.001%;
(d) comprises a cationic surfactant, and wherein the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, less than about 0.10%, greater than about 0.001%, greater than about 0.002%, greater than about 0.003%, greater than about 0.004%, greater than about 0.005%, greater than about 0.006%, greater than about 0.007%, greater than about 0.008%, greater than about 0.009%, and greater than about 0.010%; or
(e) any combination thereof.

23. The method of claim 1, wherein:
(a) the nanoemulsion comprises at least one cationic surfactant and at least one non-cationic surfactant;
(b) the nanoemulsion comprises at least one cationic surfactant and at least one non-cationic surfactant, wherein the non-cationic surfactant is a nonionic surfactant;
(c) the nanoemulsion comprises at least one cationic surfactant and at least one non-cationic surfactant, wherein the non-cationic surfactant is a polysorbate nonionic surfactant;
(d) the nanoemulsion comprises at least one cationic surfactant and at least one nonionic surfactant which is polysorbate 20 or polysorbate 80;
(e) the nanoemulsion comprises at least one cationic surfactant and at least one non-cationic surfactant, wherein the non-cationic surfactant is a nonionic surfactant, and the non-ionic surfactant is present in a concentration of about 0.05% to about 10% or about 0.1% to about 7%;
(f) the nanoemulsion comprises at least one cationic surfactant and at least one a nonionic surfactant, wherein the cationic surfactant is present in a concentration of about 0.05% to about 2%; or
(g) any combination thereof.

24. The method of claim 1, wherein the water is present in Phosphate Buffered Saline (PBS).

25. The method of claim 1, wherein:
(a) the nanoemulsion is topically or intradermally applied in a single administration;
(b) the nanoemulsion is topically applied, followed by washing the application area to remove any residual nanoemulsion;
(c) the nanoemulsion is topically or intradermally applied for at least once a week, at least twice a week, at least once a day, at least twice a day, three times a day, four times a day, multiple times daily, multiple times weekly, biweekly, at least once a month, or any combination thereof;
(d) wherein the nanoemulsion is topically or intradermally applied for a period of time selected from the group consisting of about one day, two days, three days, four days, about one week, one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years; or
(e) any combination thereof.

26. The method of claim 1, wherein:
(a) the nanoemulsion exhibits minimal systemic absorption in the human subject, meaning that less than 10 ng/ml of the surfactant is measured in the plasma of the subject;
(b) the nanoemulsion applied topically or intradermally is not systemically toxic to the human subject;
(c) following application, less than 5 ng/ml of the surfactant is measured in the plasma of the subject;
(d) following application, less than 3 ng/ml of the surfactant is measured in the plasma of the subject;
(e) following application, less than 1 ng/ml of the surfactant is measured in the plasma of the subject;
(f) following topical application of the nanoemulsion the nanoemulsion is occluded or semi-occluded;
(g) following topical application of the nanoemulsion the nanoemulsion is occluded or semi-occluded and occlusion or semi-occlusion is performed by overlaying a bandage, polyolefin film, article of clothing, impermeable barrier, or semi-impermeable barrier to the topical preparation;
(h) the nanoemulsion is topically applied in the form of an article or carrier such as a bandage, insert, syringe-like applicator, pessary, powder, talc or other solid, solution, liquid, spray, aerosol, shampoo, cleanser (leave on and wash off product) ointment, foam, cream, gel, paste, lotion, microcapsules, bioadhesive gel, or combination thereof;
(i) the nanoemulsion is topically applied using an electrophoretic device;
(j) the nanoemulsion is a controlled release formulation, sustained release formulation, immediate release formulation, or any combination thereof; or
(k) any combination thereof.

27. The method of claim 1, wherein:
(a) following treatment the mean time to healing of the lesions is decreased, as compared to a control;
(b) following treatment the mean time to healing of the lesions, as compared to a control, is decreased by at least 1 day (24 hour period);
(c) following treatment the incidence of aborted lesions is increased, as compared to a control;
(d) 3 days after initiation of treatment, the subject has complete healing of lesions;
(e) after treatment the subject does not exhibit or has reduced shedding of virus; or
(f) any combination thereof.

28. The method of claim 1, wherein the efficacy is:
(a) equivalent to or better than an orally administered drug used to treat a lesion associated with a herpes virus infection;
(b) equivalent to or better than an orally administered drug used as claimed in the product label to treat a lesion associated with a herpes virus infection;
(c) better than any commercially available topically applied drug used to treat a lesion associated with a herpes virus infection;
(d) better than any commercially available topically applied drug, as claimed in the product label, used to treat a lesion associated with a herpes virus infection; or
(e) any combination thereof.
